(19) United States
(12) Patent Application Publication
(10) Pub. No.: US 2015/0147276 A1
(43) Pub. Date: May 28, 2015

(54) NANOTHERAPEUTICS FOR DRUG TARGETING

(71) Applicant: PRESIDENT AND FELLOWS OF HARVARD COLLEGE, Cambridge, MA (US)

(72) Inventors: Donald E. Ingber, Boston, MA (US); Netanel Korin, Brookline, MA (US); Mathumai Kanapathipilai, Boston, MA (US); Oktay Uzun, Boston, MA (US); Anne-Laure Papa, Boston, MA (US)

(73) Assignee: PRESIDENT AND FELLOWS OF HARVARD COLLEGE, Cambridge, MA (US)

(21) Appl. No.: 14/405,251

(22) PCT Filed: Jun. 7, 2013
(86) PCT No.: PCT/US2013/044709

§ 371 Date: Dec. 3, 2014

Related U.S. Application Data
(60) Provisional application No. 61/656,753, filed on Jun. 7, 2012.

(51) Int. Cl. A61K 47/34; A61K 49/22; C08G 63/06; A61K 38/48

(52) U.S. Cl. CPC A61K 47/34; A61K 38/482; A61K 49/225; C08G 63/06; C12Y 304/21068
USPC 424/9.5; 424/94.3; 424/451; 514/772.3; 528/361; 435/188

(57) ABSTRACT

The invention provides compositions and methods for targeted controlled drug release. The compositions and methods can be used for treating or imaging vascular stenosis, stenotic lesions, occluded lumens, embolic phenomena, thrombotic disorders and internal hemorrhage.

### CROSS-REFERENCE TO RELATED APPLICATIONS

This application claims benefit under 35 U.S.C. §119(e) of U.S. Provisional Application No. 61/656,753, filed Jun. 7, 2012, the content of which is incorporated herein by reference in its entirety.

### FIELD OF THE INVENTION

The present invention relates to compositions and methods for targeted delivery and controlled release of therapeutics or imaging agent to a desired site. The invention also relates to compositions and methods for treating or imaging stenosis, stenotic lesions, thrombolytic therapies, and internal hemorrhage.

### BACKGROUND OF THE INVENTION

Selective delivery of drugs to defined sites of disease is one of the most promising advantages of nanoscaled drug carriers. Targeting of drugs and imaging agents is based on utilizing abnormal features of disease state such as: elevated pH in tumor, enhanced blood vessel permeability in cancer, decreased oxygen level in hypoxic regions, up-regulated cell surface antigens or molecular affinity of targeting moieties to pathological tissue. Based on these characteristics, different drug delivery schemes have been developed. Physical forces play a major role in tissue functionality and disease, however, targeting strategies based on such parameters have not been proposed.

Fluid shear stress is an important physiological feature of the blood circulation that is tightly regulated under normal physiological conditions. Shear stress has been shown to play a major role in regulating endothelial cell phenotype and gene expression, platelet and red blood cell (RBC) aggregation, arteriogenesis and hemodynamic properties. Stenosis, abnormal narrowing in blood vessels due to blockage, constriction or malformation, significantly alters the characteristics of local blood flow; differing this region from normal physiological conditions. For example, wall shear stress at atherosclerotic stenotic sites may be two orders of magnitude higher than normal physiological shear stress levels. These abnormal shear stresses induce platelet activation and facilitate thrombus formation.

As a driving force, shear can cause morphological and structural changes in single and collective elements at varying length scales. The interaction between shear stress and different forms of potential drug carriers including: nano/microspheres, microcapsules and microgels have been extensively studied. As shear increases single particles deform and eventually break. Shear triggered breakup of microcapsule/nanocapsule is being successfully employed in cosmetic products for active ingredient release upon rubbing against the skin. However, these or alternative approaches have not been suggested or developed for targeted drug delivery to sites of stenosis within the vasculature or other fluid filled channels in the body.

### SUMMARY OF THE INVENTION

In one aspect, the invention provides an aggregate, comprising a plurality of nanoparticles, wherein the aggregate disaggregates under a predetermined stimulus. The stimulus can be shear stress, physical strain, mechanical strain, ultrasound, magnetic, radiation (e.g., visible, UV, IR, near-IR, x-ray, etc. . . . ), temperature, pressure, ionic strength, pH, turbulence, change in flow, flow rate, vibrations, or chemical or enzymatic activation, and the like.

In another aspect, the invention provides a method for delivering a therapeutic agent or an imaging or contrast agent to a desired site of action a subject, the method comprising administering to a subject in need thereof an aggregate described herein.

In another aspect, the invention provides a method for treating or imaging stenosis and/or a stenotic lesion in a subject, the method comprising administering to a subject in need thereof an aggregate described herein.

In another aspect, the invention provides a method for treating or imaging a blood clot and/or an obstructive lesion in a subject, the method comprising administering to a subject in need thereof an aggregate described herein.

In yet another aspect, the invention provides a method for treating or imaging internal hemorrhage in a subject, the method comprising administering to a subject in need thereof an aggregate described herein.

In still yet another aspect, the invention provides a theranostic method, the method comprising administering to a subject in need thereof an aggregate described herein, wherein the aggregate comprises both a therapeutic agent and an imaging or contrast agent.

In some embodiments, the method according to the various aspects disclosed herein further comprises providing a stimulus to the subject to disaggregate the administered aggregate. In some embodiments the stimulus is ultrasound.

In still yet another aspect, the invention provides a kit comprising an aggregate herein or components for making an aggregate described herein.

### BRIEF DESCRIPTION OF THE DRAWINGS

FIGS. 1A-1D show microscale SA-NTs only disperse into nanoparticles when exposed to pathological shear stresses. FIG. 1A, shows scanning electron micrographs of the microscale (˜2-5 μm) SA-NTs (left) and the PLGA nanoparticles (NPs; ˜180 nm) used to produce them (right) (bar, 2 μm). FIG. 1B shows fluorescence micrographs demonstrating intact SA-NTs (top) and NPs dispersed after their exposure to 1,000 dyne/cm2 for 10 min using a rheometer (bottom) (bar, 10 μm). FIG. 1C shows quantification of release of fluorescent NPs from the SA-NTs as a function of shear revealed that exposure to pathological levels of shear (>100 dyne/cm2 for 1 min) caused large increase in the breakup of the microscale aggregates into NPs compared to physiological levels of shear (1 or 10 dyne/cm2) (*p<0.005). FIG. 1D shows CFD simulations comparing fluidic shear stress in a normalcoronary artery (left) and a stenotic vessel with a 60% lumen obstruction (right); left inset shows the corresponding angiogram of the stenotic left coronary artery in a 63 year old male patient.

FIGS. 2A-2E show shear-induced dissociation of SA-NTs and nanoparticle targeting under hemodynamic conditions in microfluidic devices. FIG. 2A is a schematic representation of a microfluidic vascular stenosis model showing how SA-NTs (large spheres) remain intact in the pre-stenotic region, but then break up into NPs (small spheres) when they flow through a constriction (90% lumen occlusion) and can accumulate in endothelial cells lining the bottom of the channel. FIG. 2B shows a photograph of the microdevice that mimics vascular stenosis fabricated in PDMS. FIG. 2C shows CFD simulations of the microfluidic device shown in FIG. 2B demonstrating that a physiological inlet shear rate of 1,000 s⁻¹ (10 dyne/cm²) upstream from the constriction increases to a pathological level of ˜100,000 s^{-1 [}1,000 dyne/cm²] in the region displaying 90% lumen occlusion. FIG. 2D is a graph showing a greater than 10-fold increase in release of fluorescent NPs from SA-NTs when they are perfused through the channel shown in FIG. 2B compared with flow through an unconstricted channel (*p<0.005). Fluorescent micrographs compare the NPs collected in the outflow from the control channel (top) versus the constricted channel (bottom) (bar, 2 μm). FIG. 2E is a graph demonstrating that many more fluorescent NPs accumulate in endothelial cells lining the downstream area (post-stenosis) of the constriction relative to an upstream area (p<0.005). Fluorescence microscopic images show cells from regions before (left) and after (right) the constriction (bar, 20 μm).

FIGS. 3A-3D show shear-targeting of a thrombolytic drug in an arterial thrombosis model using SA-NTs. FIG. 3A is a schematic representation of the experimental strategy according to an embodiment of a method described herein. Ferric chloride injury initiates formation of a thrombus (top) that grows to partially obstruct blood flow (upper middle). Intravenously injected SA-NTs dissociate into NPs at the thrombus site due to the rise in local shear stress (lower middle). Accumulation of tPA-coated NPs and binding to the clot at the occlusion site progressively dissolve the obstruction (bottom). FIG. 3B shows sequential intravital fluorescence microscopic images of a thrombus in a partially occluded mesenteric artery recorded over a 5 min period beginning after bolus injection of fluorescent tPA-coated SA-NTs (1 mg NPs; 50 ng tPA) 8 min after injury initiation (bar, 100 μm). Note that the NPs accumulate at the clot, first visualizing its location and then demonstrating clearance of the clot within 5 min after injection at the bottom. FIG. 3C shows a sequence of intravital fluorescence microscopic images recorded over a 5 min period showing fluorescently-labeled platelets accumulated within a forming thrombus that partially occludes a mesenteric artery 8 min after injury that was then treated with injection of either tPA-carrying SA-NTs (50 ng tPA) (left) or PBS (right) (bar, 100 μm). Note that the clot on the left is greatly reduced in size within 5 min after SA-NTs injection, whereas the control vessel on the right fully occludes over the same time period. FIG. 3D is graph showing that a bolus injection of SA-NTs carrying 50 ng tPA (tPA-SA-NT) significantly delayed the time to full vascular occlusion in FeCl-injured vessels (***p<0.0005), whereas administration of the same concentration of soluble tPA (free tPA), uncoated SA-NTs (bare SA-NT), tPA-coated NPs that were artificially dissociated from SA-NTs prior to injection (dispersed tPA-NPs), and heat-fused NP microaggregates with tPA coating that do not dissociate (fused SA-NT) did not produce any significant delay in thrombosis.

FIGS. 4A-4G shows shear-targeting of a thrombolytic drug to vascular emboli in vitro and therapeutic delivery in a mouse pulmonary embolism model. FIG. 4A shows time lapse fluorescence (top) and (bottom) views of artificial microemboli (˜250 μm) in a microfluidic channel before (0 min) and 1 or 60 min after injection of SA-NTs coated with tPA (50 ng/ml) showing progressive lysis of the clots over time (also see Supplementary S3 movie; bar, 100 μm). FIG. 4B is a graph showing enhanced emboli lysis kinetics induced by tPA-coated SA-NTs (50 ng/ml, blue line) compared to soluble tPA (red line). FIG. 4C are fluorescence (top) and phase contrast (bottom) views of histological sections of normal (left) versus obstructed (right) pulmonary arteries showing local accumulation of fluorescent NPs within the obstructing emboli in a mouse ex vivo lung ventilation-perfusion model (bar, 100 μm). FIG. 4D is a graph showing almost a 20-fold increase (p<0.005) in accumulation of fluorescent NPs in regions of obstructed versus non-obstructed vessels, as detected by microfluorimetry. FIG. 4E shows real-time measurements of pulmonary artery pressure in the ex vivo pulmonary embolism model showing that the tPA-coated SA-NTs (blueline) reversed pulmonary artery hypertension within approximately 1 hour, whereas the same concentration (50 ng/ml) of free tPA was ineffective (red line). FIG. 4F is a graph showing that tPA carrying SA-NTs normalize pulmonary artery pressure within an hour, whereas the same concentration of free tPA (50 ng/ml) or a 10 times higher dose (500 ng/ml) did not reduce pulmonary artery pressure (*p<0.005); only a 100-fold higher dose (5,000 ng/ml) produced similar effects. FIG. 4G shows survival curve showing that almost all (86%) of the mice injected with the tPA-coated SA-NTs survived, whereas all control mice died within 45 min after injection of fibrin clots that caused acute emboli formation.

FIGS. 5A-5C show enhanced adhesion of nanoparticles compared to microparticles under flow. FIG. 5A shows that nanoparticles (NPs) experience lower hemodynamic forces (F_{hydro}) due to their smaller size (F_{hydro}≈r²) compared to micrometer-sized particles, causing them to adhere more efficiently to the surrounding vascular wall and surface endothelium, while the larger particles that experience higher drag forces are pulled away by fluid flow. FIG. 5B shows fluorescence microscopic images showing much higher level of binding of the NPs (average size 200 nm) at the left, compared to the microaggregates (average size 2 μm) at the right. Both NP solutions were coated with tPA (50 ng/mg) and infused at the same concentration (100 μg/ml in PBS) for 15 min through a fibrin-coated 80 μm channel, which produces the same normal shear stress of 10 dyne/cm² (bar, 10 μm). FIG. 5C shows quantitation of the surface adhesion of tPA-coated NPs compared to microaggregates corresponding to the normal conditions described in FIG. 5B.

FIGS. 6A and 6B show induction of emboli lysis in vivo in the mouse pulmonary embolism model using t-PA-coated SA-NTs. Graphs (left) and fluorescence microscopic images (right) show that intravenous administration with tPA-coated SA-NTs (+SA-NTs) immediately (FIG. 6A) or 30 min (FIG. 6B) after infusion of fluorescent fibrin clots (<70 μm) and induction of multiple small emboli results in a significant (p<0.05) reduction in both the total area covered by emboli and the number of emboli in the lungs compared to controls injected with PBS. Data are presented normalized relative to control results at the left; green dots in images at right indicates fluorescent emboli; red represents a brightfield image of the lung (bar, 150 μm).

FIG. 7 shows biodistribution of SA-NTs and NPs in mice measured 5 min after intravenous administration. The SA-NTs or NPs (5 mg/ml) were injected as a bolus (100 μl) through the jugular vein of mice, and 5 min later the major organs responsible for clearance of particulates (liver, lung, spleen, and kidney) and the blood were harvested. The percentage of the Injection Dose (ID) contained within each organ (% ID/organ) was estimated based on fluorescence measurements of the harvested tissues. Note that the SA-NTs and NPs exhibited different clearance efficiencies with a much great proportion of the SA-NTs being cleared (primarily by the liver) within 5 min after injection.

FIG. 8 is a fluorescence image of RBC ghosts loaded with FITC-dextran (MW 70 kDa) taken five days from preparation.

FIG. 9 is a bar graph showing increased release from RBC ghosts flowing through a stenosis.

FIG. 10 is a bar graph showing release of FITC-dextran from Pluronic-PEI microcapsules flowing through a stenosis.

FIG. 11 is a size distribution histogram of the phosphorex based spary dried particles using Beckman Coulter counter Multisizer™ 4 with a 30 micron aperture which covers size range from 0.6 micron to 18 micron. Mean particle size of the particles is 3.8 micron with Std. Dev. 2.03. Using the instrument, particle size characterization can be carry out using only ˜0.5 mg of sample with 10 min total measurement time.

FIG. 12 is bar graph showing quantitation of release of fluorescent nanoparticles from shear activated microaggregates when exposed to agitation by therapeutic sound, US, (2 W/cm⁻², 1 MHz, 50% duty cycle) compared to when sheared at a high pathological level of shear (1,000 dyne/cm² by flowing through a 90% contraception microfluidic device, 20 min), left bar. The fluorescent intensity of the collected NP suspensions was measured using a spectrometer (Photon Technology International, NJ) and normalized relative to the results of the sheared suspension. The results show that therapeutics levels of ultrasound agitation can cause similar release of NPs as shearing at a high pathological shear stress.

FIG. 13 is a schematic representation of the PEGylation approach to graft a molecule (e.g. tPA) at the surface of the PLGA microparticles in three steps. In the first step (I), the carboxyl groups of the PLGA particles are activated by EDC/NHS chemistry. NH₂-PEG-COOH is subsequently conjugated. The second step (II) describes the activation of the PEG carboxylic groups by EDC/NHS chemistry. Amine groups of the tPA are then able to react with the activated carboxylic groups of the PEG (III).

### DETAILED DESCRIPTION OF THE INVENTION

In one aspect, the invention provides an aggregate, comprising a plurality of nanoparticles, wherein the aggregate disaggregates under a stimulus. For example, the stimulus can be an external stimulus or an internal stimulus. Exemplary stimuli can include, but are not limited to, shear stress, physical strain, mechanical strain, ultrasound, magnetic, radiation (e.g., visible, UV, IR, near-IR, x-ray, etc. . . . ), temperature, pressure, ionic strength, pH, turbulence, change in flow, flow rate, vibrations, or chemical or enzymatic activation, and the like. In some embodiments, the aggregate can be used to deliver a compound of interest, e.g., a therapeutic agent and/or an imaging agent, to a localized site where restricted and/or constrained fluid flow at the site results in elevated fluid shear stress. Without limitations, an aggregate can comprise a heterogeneous mix of nanoparticles of different types, shapes, morphologies, sizes, chemistries, therapeutic agents, imaging or contrast agents. In some embodiments of this and other aspects of the invention, the aggregate is for biomedical uses. In other embodiments of this and other aspects of the invention, the aggregate is for non-medical or industrial uses.

In some embodiments of this and other aspects of the invention, the aggregate is a micro sized aggregate. By “micro sized” is meant aggregates that are on the order of 0.1 μm to 1000 μm. The aggregate can be a regular or irregular shape. For example, the aggregate can be a spheroid, hollow spheroid, cube, polyhedron, prism, cylinder, rod, disc, lenticular, or other geometric or irregular shape. Generally, an aggregate of the invention has at least one dimension that is ≧1 μm (e.g., 1 μm or more, 2 μm or more, 5 μm or more, 10 μm or more, 20 μm or more, 30 μm or more, 40 μm or more, 50 μm or more, 60 μm or more, 70 μm or more, 80 μm or more, 90 μm or more, 100 μm or more, 150 μm or more, 200 μm or more, 250 μm or more, 300 μm or more, or 500 μm or more). In some embodiments, an aggregate has at least one dimension that is ≦500 μm (e.g., 500 μm or less, 400 μm or less, 300 μm or less, 250 μm or less, 200 μm or less, 150 μm or less, 100 μm or less, 50 μm or less, 25 μm or less, 20 μm or less, 15 μm or less, 10 μm or less, or 5 μm or less). In some embodiments, the aggregate has one dimension in the range of from about 0.5 μm to about 200 μm, preferably in the range of from about 0.75 μm to about 50 μm, more preferably in the range from about 1 μm to about 20 μm. In some embodiments, the aggregate is 1 μm to 3 μm in size. In some embodiments, the aggregate is 2.5 μm to 5.5 μm in size. In some embodiments, the aggregate is from about 1.77 to about 5.83 μm in size. In one embodiment, the aggregate is about 3.8 μm in size. In some embodiments, the aggregate is 1 μm to 10 μm in size.

Without wishing to be bound by a theory, because the aggregates of the invention are micro-sized, they can be cleared out easily in bile or, if biodegradable, they can be broken down into chemical components and passed out through the kidney. This can be advantageous for drug delivery in military and/or emergency situations. For example, the aggregates can be used for treating vascular infarction (stroke, heart attack, pulmonary embolism) because rapid occlusion of the vessels by blood clots results in a large increase in shear stress locally. The aggregates also can be used to treat bleeding. Because shear stress is high at sites of bleeding, due to high volume going through a small hole in vessel wall, the aggregates of the invention will disaggregate at the sites of bleeding. Thus, delivering pro-coagulants, which are contained in the aggregate, at the site of bleeding.

As used herein, the term “shear stress” refers to the ratio of force to area. A fluid flows in response to the applied shear force. However, when fluid flows through a channel, the fluid adjacent to the walls of the channel tends to adhere to the wall resulting in a velocity gradient. The fluid velocity increases as distance from the wall increases. The differences in fluid velocity, as indicated by the velocity gradient, result in a shear stress being applied on cells and particles flowing in the fluid. The shear stress increases as the distance to the wall decreases where the differences in fluid velocity are greater. Shear stress is also a function of radius, and thus it also increases when the channel becomes constricted. As used herein, the term “shear stress conditions” refers to conditions under which a shearing stress is applied by a fluid. The shear stress generated by the flowing fluid can be transferred or applied to molecules, particles and aggregates that may be present in the flowing fluid. These shear stress conditions can occur in a fluid having generally laminar or turbulent flow characteristics. Amount of shear stress an aggregate undergoes is a function of aggregate size.

Generally, in normal blood vessels the wall shear stress is well below 70 dyn/cm² (7Pa) while at the stenosis site shear stress exceeds 70 dyn/cm² (A M Malek, S. A. & S. Izumo “Hemodyamic shear stress and its role in atherosclerosis.” JAMA, 1999, 282: 2035-2042). Accordingly, the shear stress under which an aggregate described herein disaggregates is 5 to 3000 dyn/cm². In some embodiments, the shear stress under which an aggregate described herein disaggregates is ≧5 dyn/cm², ≧6 dyn/cm², ≧7 dyn/cm², ≧8 dyn/cm², ≧9 dyn/cm², ≧10 dyn/cm², ≧11 dyn/cm², ≧12 dyn/cm², ≧13 dyn/cm², ≧14 dyn/cm², ≧15 dyn/cm², or ≧20 dyn/cm². It is to be understood that complete disaggregation of the aggregate is not required.

The aggregate disclosed herein can disaggregate when ultrasound energy is applied to the aggregate. In some embodiments, the ultrasound intensity under which an aggregate described herein disaggregates is of low intensity. By low intensity is meant ultrasound intensity equal to or less than about 150 W/cm⁻², 125 W/cm⁻², 100 W/cm⁻², 75 W/cm⁻², 50 W/cm⁻², 25 W/cm⁻², 20 W/cm⁻², 15 W/cm⁻², 10 W/cm⁻², 7.5 W/cm⁻², 5 W/cm⁻², or 2.5 W/cm⁻². In some embodiments, the ultrasound intensity can be between 0.1 W/cm⁻² and 20 W/cm⁻²; between 0.5 W/cm⁻² and 15 W/cm⁻²; or between 1 W/cm⁻² and 10 W/cm⁻².

An aggregate described herein can disaggregate by at least 1%, at least 2%, at least 3%, at least 4%, at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, or 100% (i.e. complete disaggregation) under application of stimulus (e.g. shear stress condition, (such as a stenosis site shear stress); application of ultrasound, mechanical strain, magnetic field, radiation, or pressure; changes in temperature, ionic strength, pH, flow as compared to when the stimulus is not applied (e.g., a control shear condition (such as normal blood vessel shear stress) or absence of ultrasound, mechanical strain, magnetic field, or radiation).

The nanoparticle constituents of the aggregate can form the aggregate non-covalently or covalently. By “non-covalently” is meant that the nanoparticle constituents of the aggregate associate with each other via non-covalent means. By “covalently” is meant that the nanoparticle constituents of the aggregate associate with each other via covalent means, i.e, by a linker, e.g., a cleavable linker. Cleavable linkers are described herein below.

In some embodiments, the aggregate can comprise a matrix material for aggregating the nanoparticles. Without limitations, the aggregating matrix material can be an excipient, a therapeutic agent, a diagnostic agent, an imaging or contrast agent, a linker (e.g., a cleavable linker), or any combinations thereof.

Amount and/or rate of disaggregation can be controlled by modulating the non-covalent association of nanoparticles in the aggregate. As used herein, the term “non-covalent association” refers to an intermolecular interaction between two or more individual molecules without involving a covalent bond. Intermolecular interaction depends on, for example, polarity, electric charge, and/or other characteristics of the individual molecules, and includes, without limitation, electrostatic (e.g., ionic) interactions, dipole-dipole interactions, van der Waal's forces, and combinations of two or more thereof. Accordingly, strength of non-covalent association can be modulated by altering one or more of the above-mentioned intermolecular interactions. For example, surface of nanoparticles can be modified to modulate intermolecular electrostatic interactions, hydrogen bonding interactions, dipole-dipole interactions, hydrophilic interaction, hydrophobic interactions, van der Waal's forces, and any combinations thereof between two or more nanoparticles.

One method of controlling association strength is by including pair of affinity binding pairs on the surface of nanoparticles and modulating the intermolecular association of these affinity binding pairs by modulating one or more of the above-noted intermolecular interactions.

Rate of disaggregation can also be optimized by optimizing spray-drying conditions used for aggregation. For example, spray-drying conditions can be modulated to fine-tune disaggregation using, among others, inlet temperature, outlet temperature, atomization pressure, atomizer type, flow, solution/suspension feed rate, solvents, excipients, nozzle pressure, humidity, and the like. Exemplary excipients include, but are not limited to, leucine; lysine; sucrose; D-mannose; D-fructose; dextrose; trehalose; lactose; glucose; mannitol; sorbitol; potassium phosphate; plasdone C; anhydrous lactose; micro crystalline cellulose; polacrilin potassium; magnesium stearate; cellulose acetate phthalate; alcohol; acetone; gelatin; cellulose; cellulose derivatives; starch; polyvinylpyrrolidone; polyethylene glycol; calcium carbonate; magnesium stearate; adipic acid; ammonium chloride; butylene glycol; calcium acetate; calcium chloride; calcium hydroxide; calcium lactate; calcium silicate; cellulose (microcrystalline and carboxymethylcellulose sodium); ceresin; coconut oil; corn starch and pregelatinized starch; glycine; hydrophobic colloidal silica; hydroxypropyl betadex; lactose; lactose (monohydrate and corn starch); lactose (monohydrate and microcrystalline cellulose); lactose (monohydrate and povidone); lactose (monohydrate and powdered cellulose); maleic acid; methionine; myristyl alcohol; neotame; pentetic acid; phospholipids; poly(dl-lactic acid); polyoxylglycerides; potassium alum; propylparaben sodium; safflower oil; sodium carbonate; sodium formaldehyde sulfoxylate; sodium thiosulfate; sucrose octaacetate; sulfur dioxide; tagatose; tricaprylin; triolein; vitamin E polyethylene glycol succinate; and any combinations thereof.

As used herein, the term “hydrophilic interaction” refers to an attraction toward water molecules, wherein a material/compound or a portion thereof may bind with, absorb, and/or dissolve in water. As used herein, the term “hydrophobic interaction” refers to repulsion against water molecules, wherein a material/compound or a portion thereof does not bind with, absorb, or dissolve in water. Association strength can be controlled by modulating the hydrophilic and/or hydrophobic characteristics of nanoparticle surface. For example, more hydrophobic nanoparticles would cluster together under hydrophilic conditions (e.g. in blood). Conversely, more hydrophilic nanoparticles would not cluster together under hydrophilic conditions.

As used herein, the term “electrostatic interaction” refers to an intermolecular interaction between two or more positively or negatively charged moieties/groups, which may be attractive when two are oppositely charged (i.e., one positive, another negative), repulsive when two charges are of the same sign (i.e., two positive or two negative), or a combination thereof. Electrostatic interaction can be modulated by including positively and negatively charged moieties/groups on the surface of the nanoparticles. By adjusting the ratio of positive to negative charges strength of association of nanoparticles can be modulated; thus, controlling the rate of disaggregation.

As used herein, the term “dipole-dipole interaction” refers an intermolecular attraction between two or more polar molecules, such as a first molecule having an uncharged, partial positive end δ+ (e.g., electropositive head group such as the choline head group of phosphatidylcholine) and a second molecule having an uncharged, partial negative end δ− (e.g., an electronegative atom such as the heteroatom O, N, or S in a polysaccharide). Dipole-dipole interaction also refers to intermolecular hydrogen bonding in which a hydrogen atom serves as a bridge between electronegative atoms on separate molecules and in which a hydrogen atom is held to a first molecule by a covalent bond and to a second molecule by electrostatic forces.

As used herein, the term “hydrogen bond” refers to an attractive force or bridge between a hydrogen atom covalently bonded to a first electronegative atom (e.g., O, N, S) and a second electronegative atom, wherein the first and second electronegative atoms may be in two different molecules (intermolecular hydrogen bonding) or in a single molecule (intramolecular hydrogen bonding). Strength of association between nanoparticles can be modulated by modulating the number of intermolecular hydrogen bonds the nanoparticles can form with each other. More intermolecular hydrogen bonds leading to stronger association; thus a lower rate of disaggregation. Conversely, less intermolecular hydrogen bonds lead to a weak association; thus a higher rate of disaggregation.

As used herein, the term “van der Waal's forces” refers to the attractive forces between non-polar molecules that are accounted for by quantum mechanics. Van der Waal's forces are generally associated with momentary dipole moments induced by neighboring molecules undergoing changes in electron distribution.

In some embodiments of this and other aspects of the invention described herein, one or more compounds, e.g., a compound to be delivered, can be associated with the aggregate. As used herein, with respect to aggregates, the phrase “associated with” means entangled, embedded, incorporated, encapsulated, bound to the surface, or otherwise associated with the aggregate or a nanoparticle constituent of the aggregate.

Without wishing to be bound by a theory, the compound can be covalently or non-covalently associated with the aggregate or nanoparticle constituent of the aggregate. In some embodiments of this and other aspects of the invention described herein, the compound is encapsulated within the aggregate or a nanoparticle constituent of the aggregate.

In some embodiments of this and other aspects described herein, the molecule is non-covalently linked to with the aggregate or a nanoparticle constituent of the aggregate.

In some embodiments of this and other aspects of the invention described herein, the compound is absorbed or adsorbed on the surface of the aggregate or a nanoparticle constituent of the aggregate. Thus, a molecule can be associated with outer surface of the aggregate. This can result from when only the nanoparticle on the outer surface of the aggregate are associated with the molecule. For example, the aggregate can be fabricated and the associated with the molecule.

In some embodiments of this and other aspects of the invention described herein, the molecule or compound is covalently linked with the aggregate or a nanoparticle constituent of the aggregate.

It is to be understood that a compound does not need to be associated with a nanoparticle while the compound is in the aggregate. For example, preformed nanoparticle can be aggregated in the presence of the compound. Without wishing to be bound by a theory, the compound can then be present in the spaces (or cavities) in the aggregate.

In some embodiments of this and other aspects of the invention, the aggregate comprises at least two or more therapeutic agents. For a non-limiting example, the aggregate can comprise two or more different therapeutic agents that are known in the art to treat a disease, disorder, or condition.

In some embodiments of this and other aspects of the invention, the aggregate comprises an inflammatory agent and another therapeutic agent. The other therapeutic may or may not be an inflammatory agent.

In some embodiments of this and other aspects of the invention, the aggregate comprises at least one therapeutic agent and at least one diagnostic, imaging or contrast agent. This can be useful in theranostics. In some embodiments, the therapeutic agent is tPA and the imaging or contrast agent is a fluorescent dye (e.g. coumarin).

In some embodiments of this and other aspects of the invention, the aggregate comprises at least one therapeutic agent and at least one diagnostic, imaging or contrast agent, wherein the therapeutic agent and the diagnostic, imaging or contrast agent are both independently a monoclonal antibody or fragment thereof or a polyclonal antibody or fragment thereof.

In some embodiments of this and other aspects of the invention, the aggregate comprises at least one therapeutic agent and at least one diagnostic, imaging or contrast agent, and/or one targeting agent wherein the therapeutic agent and the diagnostic, imaging or contrast agent are both independently a monoclonal antibody or fragment thereof or a polyclonal antibody or fragment thereof.

In some embodiments of this and other aspects of the invention, the aggregate comprises at least one therapeutic agent and at least one diagnostic, imaging or contrast agent, and one targeting agent wherein the therapeutic agent and the diagnostic, imaging or contrast agent and the targeting ligand are independently a monoclonal antibody or fragment thereof or a polyclonal antibody or fragment thereof.

In some embodiments of this and other aspects of the invention, the aggregate comprises at least one therapeutic agent and at least one targeting agent wherein the therapeutic agent and targeting agent are both independently a monoclonal antibody or fragment thereof or a polyclonal antibody or fragment thereof.

In some embodiments of this and other aspects of the invention, the aggregate or the nanoparticle constituent of the aggregate can be coated with a zwitter ion. Without wishing to be bound by a theory, the zwitter ion coating can reduce or inhibit non-specific binding of the aggregate or the nanoparticle constituent of the aggregate. The term “zwitter ion” refers to a compound that is electrically neutral but carries formal positive and negative charges. Exemplary zwitter ions include, but are not limited to, betaine derivatives (such as sulfobetaines, e.g., 3-(trimethylammonium)-propylsulfonat or phosphobetaines), tricine, bicine, glycilglycine, TAPS, EPPS, glycine, proline, zwitterionic polymers and copolymers, zwitterionic phosphohpids, and the like.

### Nanoparticles

As used herein, the term “nanoparticle” refers to particles that are on the order of 10⁻⁹ or one billionth of a meter and below 10⁻⁶ or 1 millionth of a meter in size. The term “nanoparticle” includes nanospheres; nanorods; nanoshells; and nanoprisms; and these nanoparticles may be part of a nanonetwork. The nanoparticle can be a regular or irregular shape. For example, the nanoparticle can be a spheroid, hollow spheroid, cube, polyhedron, prism, cylinder, rod, disc, lenticular, or other geometric or irregular shape. The term “nanoparticles” also encompasses liposomes and lipid particles having the size of a nanoparticle. The particles may be, e.g., monodisperse or polydisperse and the variation in diameter of the particles of a given dispersion may vary, e.g., particle diameter of between about 0.1 to 100 nm.

As used herein, the term “liposome” encompasses any compartment enclosed by a lipid bilayer. Liposomes may be characterized by membrane type and by size. Liposomes are also referred to as lipid vesicles in the art. In order to form a liposome the lipid molecules comprise elongated non-polar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane.

Liposomes include unilamellar vesicles, which are comprised of a single lipid layer and generally have a diameter of 20 to 100 nanometers; large unilamellar vesicles (LUVS) are typically larger than 100 nm, which can be produced by subjecting multilamellar liposomes to ultrasound. Preferred liposomes have a diameter in the range of 20-250 nm.

Without limitation, there are at least ten types of nanoparticles that can be used in forming the aggregates of the invention: (1) nanoparticles formed from a polymer or other material to which a molecule of interest, e.g., a therapeutic agent, an imaging agent or a ligand, absorbs/adsorbs or forms a drug coating on a nanoparticle core; (2) nanoparticles formed from a core formed by the molecule of interest, e.g., a therapeutic agent, an imaging agent or a ligand, which is coated with a polymer or other material; (3) nanoparticles formed from a polymer or other material to which a molecule of interest, e.g., a therapeutic agent, an imaging agent or a ligand, is covalently linked; (4) nanoparticles formed from molecule of interest (e.g., a therapeutic agent, an imaging agent or a ligand) and other molecules; (5) nanoparticles formed so as to comprise a generally homogeneous mixture of a therapeutic agent, an imaging agent or a ligand with a constituent of the nanoparticle or other non-drug substance; (6) nanoparticles of pure drug or drug mixtures with a coating over a core of a molecule of interest, e.g., a therapeutic agent, an imaging agent or a ligand; (7) nanoparticles without any associated compound of interest; (8) nanoparticles composed entirely of a therapeutic agent, an imaging agent or a biologically active compound; (9) nanoparticle which have a molecule of interest, e.g., a therapeutic agent, an imaging agent or a ligand, permeated in the nanoparticles; and (10) nanoparticles which have a molecule of interest, e.g., a therapeutic agent, an imaging agent or a ligand, adsorbed to the nanoparticles.

In some embodiments, the compound of interest, e.g., a therapeutic agent, an imaging agent or a ligand, is coated on the outer surface of the aggregate, i.e., a compound of interest forms a coating on the outer surface of the aggregate. Without wishing to be bound by a theory, a subset of the nanoparticles present in the aggregate comprise a compound of interest on the surface (i.e., the surface is coated with the compound of interest) and these nanoparticles are then present the compound of interest on the outer surface of the aggregate.

In some embodiments, the outer surface of the aggregate can be coated with a compound of interest after forming the aggregate with the nanoparticles. For example, ligands and/or chemically reactive groups can be present on the outer surface of the nanoparticles in the aggregate, and these ligands and/or chemical groups can be utilized to couple a compound of interest to the outer surface of the aggregate.

In some embodiments, a compound of interest can be absorbed/adsorbed on the outer surface of a preformed aggregate in order to form a coating of the compound of interest on the outer surface of the aggregate.

It is not necessary for every nanoparticle in the aggregate to comprise a compound of interest. Only a subset of the nanoparticles may comprise a compound of interest. For example, in an aggregate at least 2%, at least 5%, at least 10%, at least 20%, at least 30%, at least 40%, at least 50%, at least 60%, at least 70%, at least 80%, at least 90%, at least 95%, or 100% (i.e. all of the nanoparticles) can comprise a compound of interest. In some embodiments, not all of the nanoparticles comprise a compound of interest.

A skilled artisan is well aware of a wide variety of nanoparticles for drug delivery that are known in the art. Accordingly, nanoparticles amenable to the invention include those described, for example, in U.S. Pat. No. 6,645,517; No. 5,543,158; No. 7,348,026; No. 7,265,090; No. 7,541,046; No. 5,578,325; No. 7,371,738; No. 7,651,770; No. 9,801,189; No. 7,329,638; No. 7,601,331; and No. 5,962,566, and U.S. Pat. App. Pub. No. US2006/0280798; No. US2005/0281884; No. US2003/0223938; 2004/0001872; No. 2008/0019908; No. 2007/0269380; No. 2007/0264199; No. 2008/0138430; No. 2005/0003014; No. 2006/0127467; No. 2006/0078624; No. 2007/0243259; No. 2005/0058603; No. 2007/0053870; No. 2006/0105049; No. 2007/0224277; No. 2003/0147966; No. 2003/0082237; No. 2009/0226525; No. 2006/0233883; No. 2008/0193547; No. 2007/0292524; No. 2007/0014804; No. 2004/0219221; No. 2006/0193787; No. 2004/0081688; No. 2008/0095856; No. 2006/0134209; and No. 2004/0247683, content of all of which is incorporated herein by reference.

In some embodiments of this and other aspects of the invention, nanoparticle is a Perflubutane Polymer Microsphere or HDDS™ (Hydrophobic Drug Delivery System) from Acusphere (www.acusphere.com/technology/home.html). Perflubutane Polymer Microspheres are made by creating an emulsion containing PLGA (polylactic-co-glycolic acid), a phospholipid and a pore-forming agent. This emulsion is further processed by spray drying to produce small, porous microspheres containing gas analogous in structure of honeycombs.

Without wishing to be bound by a theory, HDDS™ can convert a broad class of drugs that do not dissolve well in water, or hydrophobic drugs, into microspheres or nanospheres of the drug embedded in small microspheres that can more rapidly dissolve in water. One preferred HDDS™ is AI-850™, which is a reformulation of the hydrophobic drug paclitaxel and is bioequivalent to Abraxis Bioscience's ABRAXANE®, a leading cancer drug. This can be delivered to inhibit intimal hyperplasia or vascular constriction due to cell overgrowth.

In some embodiments of this and other aspects of the invention, the nanoparticles have an average diameter of from about 10 nm to about 500 nm. In some embodiments, the nanoparticles have an average diameter of from about 50 nm to about 250 nm. In one embodiment, the nanoparticles have an average diameter of from about 100 nm to about 250 nm. In one embodiment, the nanoparticles have an average diameter of about 180 nm.

Without limitation, nanoparticles amenable to the invention can be composed of any material. In some embodiments of this and other aspects of the invention, the nanoparticle comprises a polymer, e.g. a biocompatible polymer. The average molecular weight of the polymer, as determined by gel permeation chromatography, can range from 20,000 to about 500,000.

As used herein, the term “biocompatible” means exhibition of essentially no cytotoxicity or immunogenicity while in contact with body fluids or tissues. As used herein, the term “polymer” refers to oligomers, co-oligomers, polymers and co-polymers, e.g., random block, multiblock, star, grafted, gradient copolymers and combination thereof.

The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also noninflammatory when employed in situ.

Biodegradable polymers are disclosed in the art. Examples of suitable biodegradable polymers include, but are not limited to, linear-chain polymers such as polypeptides, polynucleotides, polysaccharides, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), fumaric acid, sebacic acid, and copolymers, terpolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), Poloxamers (e.g. Pluronic such as Poloxamers 407 and 188), Hyaluron, heparin, agarose, Pullulan, and copolymers including one or more of the foregoing, such as ethylene/vinyl alcohol copolymers (EVOH).

In some embodiments, the biocompatible polymer is a copolymer of polylactic acid and polyglycolic acid, poly(glycerol sebacate) (PGS), poly(ethylenimine), Pluronic (Poloxamers 407, 188), Hyaluron, heparin, agarose, or Pullulan.

In some embodiments, the polymer is a copolymer of fumaric/sebacic acid.

In some embodiments, the nanoparticle is non-polymer nanoparticle. A non-polymer nanoparticle can be a metal nanoparticle. In one embodiment, the nanoparticle is a gold nanoparticle.

In addition to the molecule of interest, the aggregate or nanoparticle constituent of the aggregate can comprise additional moieties that can extend the in vivo lifetime of the nanoparticles or the aggregate in the blood. For example, the aggregate or nanoparticle constituent of the aggregate can comprise functional moieties that enhance the in vivo lifetime of the aggregate or nanoparticle constituent of the aggregate in the blood. The aggregate or nanoparticle constituent of the aggregate can be coated with the functional moiety. By “coated” is meant the functional moiety can be present on an outer surface. In some embodiments, each nanoparticle constituent of the aggregate can comprise the functional moiety.

One exemplary moiety for increasing the in vivo lifetime is polyethylene glycol. Accordingly, the aggregate can comprise nanoparticles which are polyethylene glycoated on the surface. In some embodiments, the functional moiety can alter the biodistribution of the nanoparticle or the aggregate.

In some embodiments, the functional moiety can be a molecule that allows self vs non-self distinction in vivo. For example, the functional moiety can be a molecule that is recognized as a self molecule in vivo. Without wishing to be bound by a theory, a molecule recognized as self does not initiate an immune response and/or clearance of the molecule. The self molecule can interact with a receptor or molecule in vivo that can identify it as a self molecule. An aggregate comprising such a self molecule would also be considered as self and its clearance inhibited or decreased.

In some embodiments, the functional moiety is CD47 or a fragment thereof. The fragment can be such that is identified as a self molecule in vivo. The CD47 or the fragment thereof can interact with a receptor on the surface of a macrophage to indicate “self” and thereby inhibiting endocytosis of the aggregate or a nanoparticle constituent of the aggregate by the macrophage. The aggregate can comprise one (or more) CD47 or a fragment thereof. In some embodiments, at least a portion of the nanoparticle constituents of the aggregate can comprise one (or more) CD47 or a fragment thereof.

### Red Blood Cells

While various aspects of the invention are discussed in relation to aggregates, one can also use red blood cells (RBCs) in place of the aggregates wherein a compound of interest, e.g., a therapeutic agent and/or an imaging agent, is associated with a red blood cell. Inventors have discovered that compounds encapsulated in red blood cells can be preferentially released from the red blood cells under shear stress. Accordingly, in one aspect, the invention provides a method for treating or imaging a stenosis, a stenotic lesion, a blood clot, an obstructive lesion, and/or an internal hemorrhage in a subject, the method comprising administering to a subject in need thereof a red blood cell, wherein the red blood cell comprises a therapeutic agent and/or an imaging agent.

There are two major approaches for the association between a compound and RBCs. The most widely used approach is compound encapsulation in RBCs using one of several encapsulation methods. The second approach is reversible or irreversible compound attachment to RBC membrane. Accordingly, in some embodiments, the compound of interest (e.g., a therapeutic agent and/or an imaging agent) is encapsulated in the RBCs. Thus, there is no covalent linking between the compound of interest and RBC membrane.

Red blood cells are the most common cells of blood, are responsible for oxygen transport and have a typical biconcave shape. Normal human RBCs have a diameter of 7-8 μm and an average volume of 90 fl. In mammals, RBCs are enucleated and lose their organelles during maturation. A human body is commonly endowed with 2-3×10¹³ RBCs continuously produced at a rate of 2 million per second. RBCs spent their 100-120 day life-span travelling the circulatory system before being selectively removed by macrophages in the reticuloendothelial system (RES).

The surface area of mature, biconcave RBCs is about 136 μm² but can swell to a sphere of approx 150 fl. It is noteworthy that RBCs can also cross undamaged capillaries of 2-3 μm in diameter. The RBC membrane is strictly connected with the membrane skeletal proteins which are organized in a uniform shell. The RBC shape can undergo a number of reversible transformations. An important determinant of RBC survival is its deformability. Key factors affecting deformability are internal viscosity (mainly contributed by RBC hemoglobin), the surface/volume of the cell and the intrinsic deformability of the membrane. The RBCs have other very interesting properties namely they behave as an osmometer since they shrink when placed into a hypertonic solution or swell when placed into a hypotonic solution. The RBCs can reach a critical hemolytic volume giving rise to holes on the membrane ranging from 10 nm up to 500 nm. These processes are usually reversible and following haemolysis the holes close and the cell resumes its biconcave shape.

Red blood cells are biocompatible carriers because they are completely biodegradable without generation of toxic products and show high biocompatibility especially when autologous erythrocytes are employed. They can be easily handled ex vivo by means of several techniques for the encapsulation of different molecules, after which one can obtain loaded erythrocytes with morphological, immunological and biochemical properties similar to those of native cells. Lacking a nucleus and other organelles, most of their volume is available for the encapsulation of drugs. They protect the encapsulated substance from premature inactivation and degradation by endogenous factors and, at the same time, the subject against the toxic effects of the drugs thus avoiding immunological reactions. Potentially a wide variety of chemicals can be encapsulated. They have a longer life-span in circulation as compared to other synthetic carriers and can act as bioreactors due to the presence of several enzymatic activities that can directly affect the loaded molecules and, in the case of loaded prodrugs, give rise to the active drug itself.

Without limitations, red blood cells can include autologous red blood cells, i.e., a cell or cells taken from a subject who is in need of treatment (i.e., the donor and recipient are the same individual). Autologous red blood cells have the advantage of avoiding any immunologically-based rejection of the cells. Alternatively, the cells can be heterologous, e.g., taken from a donor. The second subject can be of the same or different species. Typically, when the cells come from a donor, they will be from a donor who is sufficiently immunologically compatible with the recipient, i.e., will not be subject to transplant rejection, to lessen or remove the need for immunosuppression. In some embodiments, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994). In some embodiments, red blood cells are recombinant red blood cells or red blood cell derived vesicles, for example those described in U.S. Pat. No. 7,521,174 and U.S. Pat. App. Pub. No. 2009/0274630, content of both of which is incorporated herein by reference.

A number of different methods can be used to load or encapsulate a compound of interest into RBCs. Some of these methods have a physical nature (e.g., osmosis-based and electrical pulse methods) or a chemical nature (e.g., chemical perturbation of the membrane).

The methods most widely used for erythrocyte loading are commonly based on the remarkable property of the RBCs to increase in volume when placed under condition of reduced osmotic pressure, such as in the presence of a hypotonic solution. Accordingly, osmosis-based methods constitute the more standard methods for the encapsulation compounds in red blood cells. Although in terms of methodology there are differences between one method and another, they are all based on the swelling of the cells accompanied by an increase in the permeability of the membrane of the erythrocytes when it is exposed to a hypotonic solution. The encapsulation of the substance is favored because pores appear in the membrane when red cells are under reduced osmotic pressure conditions. There are several variations to these methods, such as hypotonic dilution, hypotonic pre-swelling, the osmotic pulse, hypotonic hemolysis, and hypotonic dialysis, with the latter being the one most commonly used.

Three variations of the hypotonic haemolysis procedures are available: the dilutional, preswell dilutional and dialysis methods. Generally, the hypotonic dialysis method is used because it preserves the biochemical and physiological characteristics of the RBCs resulting from the process and it results in the highest percentage of encapsulation.

In hypotonic dialysis, the suspension of erythrocytes with a suitable hematocrit is placed in a dialysis bag facing a hypo-osmotic buffer at 4° C. with osmolalities that range from 100 mosM/kg in dog erythrocytes to 200-220 mosM/kg in sheep. Generally, for human erythrocytes recommended osmolality is a about 120 mosM/kg. The osmolality of the medium implies a compromise between the efficiency of the encapsulation and the least possible hemolysis of the dialysed erythrocytes. The compound to be encapsulated tends to be included in the suspension of red cells inside the dialysis bag. Although varying in its composition, the hypo-osmotic buffer usually includes NaH₂PO₄, CO₃HNa, glucose, reduced glutathione and ATP at pH 7.4. The ATP and reduced glutathione can be added to the dialysis buffer in order to preserve the cellular energy and reduce the environment inside the red cell, respectively. The time of dialysis can vary between 20 and 180 min. In order to perform hypo-osmotic dialysis, a continuous flow dialysis device as described in C. Ropars, G. Avenard and M. Chassaigne. In: Methods in Enzymology, vol. 149, R. Green and K. J. Widder, Editors, Academic Press, San Diego (1987), pp. 242-248 can be used.

Subsequently, an annealing process is performed with the loaded erythrocytes in an isoosmotic medium for 10 min at 37° C. Finally, a resealing of the erythrocytes is performed at 37° C. using a hyperosmotic buffer. The hyperosmotic buffer usually contains adenine, inosine, glucose, pyruvate, NaH₂PO₄ and NaCl at pH 7.4. Upon conclusion of the encapsulation process, the erythrocytes are again suspended in autologous plasma for subsequent administration.

When hypo-osmotic dialysis is used, several factors can affect the performance of the encapsulation, namely the tonicity of the solutions employed, times of dialysis, pH of the medium, temperature, concentration of the drug or peptide in contact with the erythrocytes, etc. The procedure permits the encapsulation of approximately 40-50% of the added compound. The final intracellular concentration of the compound is similar to the extra-cellular concentration.

Furthermore, ZnCl₂ can be externally added to loaded RBCs. Without wishing to be bound by a theory, this induces the reversible clusterization of the band 3 protein (an anion transporter on the RBC surface). By varying the amount of Zn²⁺ used, it can be possible to modulate the in vivo survival of the treated cells by controlling the extension of band 3 clustering.

The osmotic pulse method is a variation of the osmotic-based methods that uses dimethyl sulphoxide (DMSO) to facilitate the access of the substance into the erythrocytes. The mechanism is a transient osmotic gradient across the red cell membrane with a resultant loading of drug into the erythrocyte. Use of osmotic pulse is described, for example, in R. Franco, R. Barker and M. Weiner, Adv. Biosci. (series) 67 (1987), pp. 63-72, content of which is incorporated herein by reference.

Use of hypotonic hemolysis is described, for example, in S. Jain and N. K. Jain, Indian J. Pharm. Sci. 59 (1997), pp. 275-281; G. M. Ihler and H. C. W. Tsong, Methods Enzymol. (series) 149 (1987), pp. 221-229; and G. M. Ihler, Pharmacol. Ther. 20 (1983), pp. 151-169, content of all of which is incorporated herein by reference.

Use of hypotonic dilution is described, for example, in D. A. Lewis and H. O. Alpar, Int. J. Pharm. 22 (1984), pp. 137-146; U. Zimmermann, In: Targeted Drugs, E. P. Goldberg, Editor, John Wiley & Sons, New York (1983), pp. 153-200; V. Jaitely et al., Indian Drugs 33 (1996), pp. 589-594; S. J. Updike and R. T. Wakamiya, J. Lab. Clin. Med. 101 (1983), pp. 679-691; D. A. Lewis, Pharm. J. 233 (1984), pp. 384-385; K. Adriaenssenset al. al., Clin. Chem. 22 (1976), pp. 323-326; R. Baker, Nature 215 (1967), pp. 424-425; G. M. Ihler and H. C. W. Tsong, Methods Enzymol. (series) 149 (1987), pp. 221-229; S. J. Updike, R. T. Wakarniya and E. N. Lightfoot, Science 193 (1976), pp. 681-683; N. Talwar and N. K. Jain, Drug Devel. Ind. Pharm. 18 (1992), pp. 1799-1812; E. Pitt et al., Biochem. Pharmacol. 22 (1983), pp. 3359-3368; G. M. Iher, R. M. Glew and F. W. Schnure, Proc. Natl. Acad. Sci. U.S.A. 70 (1973), pp. 2663-2666; J. R. Deloach and G. M. Ihler, Biochim. Biophys. Acta 496 (1977), pp. 136-145; and S. Bhaskaran and S. S. Dhir, Indian J. Pharm. Sci. 57 (1995), pp. 240-242, content of all of which is incorporated herein by reference.

Use of hypotonic dialysis is described, for example, in U. Zimmermann, In: Targeted Drugs, E. P. Goldberg, Editor, John Wiley & Sons, New York (1983), pp. 153-200; V. Jaitely et al., Indian Drugs 33 (1996), pp. 589-594; H. G. Erchler et al., Clin. Pharmacol. Ther. 40 (1986), pp. 300-303; G. M. Ihler and H. C. W. Tsong, Methods Enzymol. (series) 149 (1987), pp. 221-229; U. Benatti et al., Adv. Biosci. (series) 67 (1987), pp. 129-136; R. Kravtozoff et al., J. Pharm. Pharmacol. 42 (1990), pp. 473-476; J. D. Berman, Adv. Biosci. (series) 67 (1987), pp. 145-152; J. R. Deloach et al., Adv. Biosci. (series) 67 (1987), pp. 183-190; J. R. Deloach and G. M. Ihler, Biochim. Biophys. Acta 496 (1977), pp. 136-145; M. Jradeet al., Adv. Biosci. (series) 67 (1987), pp. 29-36; A. Zanella et al., Adv. Biosci. (series) 67 (1987), pp. 17-27; G. Fiorelli et al., Adv. Biosci. (series) 67 (1987), pp. 47-54; and M. I. Garin et al., Pharm. Res. 13 (1996), pp. 869-874, content of all of which is incorporated herein by reference.

Use of hypotonic preswelling is described, for example, in V. Jaitely et al., Indian Drugs 33 (1996), pp. 589-594; S. Jain and N. K. Jain, Indian J. Pharm. Sci. 59 (1997), pp. 275-281; H. O. Alpar and W. J. Irwin, Adv. Biosci. (series) 67 (1987), pp. 1-9; N. Talwar and N. K. Jain, J. Control. Release 20 (1992), pp. 133-142; D. J. Jenner et al., Br. J. Pharmacol. 73 (1981), pp. 212P-213P; H. O. Alpar and D. A. Lewis, Biochem. Pharmacol. 34 (1985), pp. 257-261; G. M. Ihler and H. C. W. Tsong, Methods Enzymol. (series) 149 (1987), pp. 221-229; E. Pitt, D. A. Lewis and R. Offord, Biochem. Pharmacol. 132 (1983), pp. 3355-3358; N. Talwar and N. K. Jain, Drug Devel. Ind. Pharm. 18 (1992), pp. 1799-1812; E. Pitt et al., Biochem. Pharmacol. 22 (1983), pp. 3359-3368; S. Jain, S. K. Jain and V. K. Dixit, Drug Devel. Ind. Pharm. 23 (1997), pp. 999-1006; H. Tajerzadeh and M. Hamidi, Drug Devel. Ind. Pharm. 26 (2000), pp. 1247-1257; M. Hamidi et al., Drug Deliv. 8 (2001), pp. 231-237; and J. Bird, R. Best and D. A. Lewis, J. Pharm. Pharmacol. 35 (1983), pp. 246-247, content of all of which is incorporated herein by reference.

Compounds can also be encapsulated in red blood cells by exposing the cells to membrane active drugs such as primaquine, hydrocortisone, vinblastine and chlorpromazine, which are known to induce stomatocyte formation in the cell membrane. Use of chemical perturbation is described, for example, in U. Zimmermann, In: Targeted Drugs, E. P. Goldberg, Editor, John Wiley & Sons, New York (1983), pp. 153-200; J. Connor and A. J. Schroit, Adv. Biosci. (series) 67 (1987), pp. 163-171; I. Ben-Bassat, K. G. Bensch and S. L. Schrier, J. Clin. Invest. 51 (1972), pp. 1833-1844; L. M. Matovcik, I. G. Junga and S. L. Schrier, Drug-induced endocytosis of neonatal erythrocytes. Blood 65 (1985), pp. 1056-1063; S. L. Schrier, A. Zachowski and P. F. Devaux, Blood 79 (1992), pp. 782-786; and M. Tonetti et al., Eur. J. Cancer 27 (1991), pp. 947-948, content of all of which is incorporated herein by reference.

Electroporation is based on inducing pores in the red blood cell membrane by exposing the cells to a strong external electrical field. These pores are able to admit compounds of different size. This method of encapsulation is a good alternative to other commonly employed techniques and has been used in the encapsulation of enzymes such as alcohol and aldehyde dehydrogenase and drugs such as diclofenac sodium. Use of electroporation dilution is described, for example, in D. A. Lewis and H. O. Alpar, Int. J. Pharm. 22 (1984), pp. 137-146; U. Zimmermann, In: Targeted Drugs, E. P. Goldberg, Editor, John Wiley & Sons, New York (1983), pp. 153-200; V. Jaitely et al., Indian Drugs 33 (1996), pp. 589-594; D. A. Lewis, Pharm. J. 233 (1984), pp. 384-385; K. Kinosita and T. Y. Tsong, Nature 272 (1978), pp. 258-260; S. Jain, S. K. Jain and V. K. Dixit, Indian Drugs 32 (1995), pp. 471-476; C. A. Kruse et al., Adv. Biosci. (series) 67 (1987), pp. 137-144; U. Zimmermann, F. Riemann and G. Pilwat, Biochim. Biophys. Acta 436 (1976), pp. 460-474; D. H. Mitchell, G. T. James and C. A. Kruse, Biotechnol. Appl. Biochem. 12 (1990) (3), pp. 264-275; T. Y. Tsong, Biophys. J. 60 (1991), pp. 297-306; C. Lizano et al., Biochim. Biophys. Acta 1425 (1998), pp. 328-336; C. Lizano, M. T. Perez and M. Pinilla, Life Sci. 68 (2001), pp. 2001-2016; Q. Dong and W. Jin, Electrophoresis 22 13 (2001), pp. 2786-2792; M. Haritou et al., Clin. Hemorheol. Microcirc. 19 (1988), pp. 205-217; and P. C. Mangal and A. Kaur, Indian J. Biochem. Biophys. 28 (1991), pp. 219-221, content of all of which is incorporated herein by reference.

Methods for encapsulating a compound of interest in RBCs are also described, for example, in L. Rossi, S. Serafini and M. Magnani, In: M. Magnani, Editor, Erythrocytes Engineering for Drug Delivery and Targeting, M. Magnani, Editor, Kluwer Academic/Plenum Publishers, New York (2003), pp. 1-18; C. Lizano, M. T. Perez and M. Pinilla, Life Sci. 68 (2001), pp. 2001-2016; R. S. Franco et al., Transfusion 30 (1990), pp. 196-200; M. Ihler, Bibl. Haematol. 51 (1985), pp. 127-133; G. M. Ihler and H. C. W. Tsong, Methods Enzymol. (series) 149 (1987), pp. 221-229; S. E. Mulholland et al., Pharm. Res. 16 (1999) (4), pp. 514-518; and L. A. Lotero, G. Olmos and J. C. Diez, Biochim. Biophys. Acta 1620 (2003) (1-3), pp. 160-166, content of all of which is incorporated herein by reference.

A number of active substances have been encapsulated into RBCs. See for example, M. Magnan et al., Drug Deliv. 2 (1995), pp. 57-61; U. Benatti et al., Biochem. Biophys. Res. Commun. 220 (1996), pp. 20-25; A. Fraternale, L. Rossi and M. Magnani, Biochem. Biophys. Acta 1291 (1996), pp. 149-154; L. Rossi et al., AIDS Res. Hum. Retroviruses 15 (1999), pp. 345-353; M. Magnani et al., Proc. Natl. Acad. Sci. U.S.A. 93 (1996), pp. 4403-4408; L. Rossi et al., AIDS Res. Hum. Retroviruses 14 (1998), pp. 435-444; L. Rossi et al., J. Antimicrob. Chemother. 47 (2001), pp. 819-827; P. Franchetti et al., Antivir. Chem. Chemother. 12 (2001), pp. 151-159; P. Franchetti et al., Antivir. Res. 47 (2000), pp. 149-158; M. D'Ascenzo et al., In: Erythrocytes as Drug Carriers in Medicine, U. Sprandel and J. L. Way, Editors, Plenum Press, New York (1997), pp. 81-88; R. Crinelli et al., Blood Cells Mol. Diseases 26 (2000), pp. 211-222; L. Rossi et al., Biotechnol. Appl. Biochem. 33 (2001), pp. 85-89; L. Rossi et al., Blood Cells Mol. Diseases 33 (2004), pp. 57-63; R. Kravtzoff et al., In: Advances in the Biosciences, R. Green and J. R. De Loach, Editors, Pergamon Press, Oxford (1991), pp. 127-137; M. Magnani et al., Biotechnol. Appl. Biochem. 18 (1993), pp. 217-226; M. Magnani et al., Alcohol Clin. Exp. Res. 13 (1989), p. 849; L. Rossi et al., In: Resealed Erythrocytes as Carriers and Bioreactors, R. Green and J. R. De Loach, Editors, Pergamon Press, Oxford (1991), pp. 169-179; L. Rossi et al., J. Antimicrob. Chemother. 53 (2004), pp. 863-866; C. De Chastellier, T. Lang and L. Thilo, Eur. J. Cell. Biol. 68 (1995), pp. 167-182; A. Antonell et al., Br. J. Haematol. 104 (1999), pp. 475-481; A. Fraternale et al., Antivir. Res. 56 (2002), pp. 263-272; A. T. Palamara et al., AIDS Res. Hum. Retroviruses 12 (1996), pp. 1373-1381; A. Fraternale et al., J. Antimicrob. Chemother. 52 (2003), pp. 551-554; R. Buhl, et al., Lancet 2 (1989), pp. 1294-1298; F. J. Staal et al., AIDS Res. Hum. Retroviruses 8 (1992), pp. 305-311; S. Mihm et al., FASEB J. 9 (1995), pp. 246-252; E. Garaci et al., Biochem. Biophys. Res. Commun. 188 (1992), pp. 1090-1096; A. T. Palamara et al., Antivir. Res. 27 (1995), pp. 237-253; A. T. Palamara et al., AIDS Res. Hum. Retroviruses 12 (1996), pp. 1537-1541; M. Magnani et al., AIDS Res. Hum. Retroviruses 13 (1997), pp. 1093-1099; Y. Murata et al., Int. Immunol. 14 (2002), pp. 201-212; M. Egholm et al., Nature 365 (1993), pp. 566-568; P. Wittung et al., FEBS Lett. 365 (1995), pp. 27-29; L. Chiarantini et al., Biochemistry 41 (2002), pp. 8471-8477; and H. Arima et al., J. Pharm. Sci. 86 (1997), pp. 1079-1084, content of all of which is incorporated herein by reference. In most of these references the drug is encapsulated as a non-diffusible pro-drug that is converted into a diffusible drug by RBC resident enzymes and released in circulation. Alternatively the drug is maintained into the RBCs until these are targeted to and phagocytised by macrophages where their content is released. In some instances, RBCs are used as circulating bioreactors for the degradation of metabolites or xenobiotics. In this case an enzyme is encapsulated into RBCs where it remains catalytically active as long as the cell circulates. These modified RBCs are able to perform as circulating bioreactors when a metabolite, and/or a xenobiotic able to cross the RBC membrane reach the enzyme within the cell.

In addition to association with a compound of interest, e.g., a therapeutic agent and/or an imaging agent, a wide variety of entities, e.g., ligands, can also be coupled to the red blood cells. These ligands can be attached to the red blood cell membrane using methods known in the art. For example, coupling of a ligand to RBC can be using a non-specific chemical cross-linkers such as tannic acid and chromium chloride. See, for example, V. R. Muzykantov et al., Anal Biochem. (1993) 208:338-342; V. R. Muzykantov et al., Am J Pathol. (1987) 128:276-285; and L. Chiarantini et al., Biotechnol Appl Biochem. (1992), 15:171-184, content of all of which is incorporated herein by reference. Alternatively, coupling of a ligand to RBC can be using specific cross-linkers for coupling to defined reactive groups on RBC membrane. In particular, controlled biotinylation of RBC lysine residues using NHS esters of biotin is one of the most popular means for conjugation cargoes to RBC surface for a wide variety of applications in vitro and in vivo. Use of specific cross-linkers for linking molecules to RBC is described, for example, in G. A. Orr G A, J Biol Chem. (1981) 256:761-766; W. Godfrey et al., Exp Cell Res. (1981) 135:137-145; E. Roffman et al., Biochem Biophys Res Commun. (1986) 136:80-85; E. A. Bayer et al., Anal Biochem. (1987) 161:262-271; M. Wilchek et al., Biochem Biophys Res Commun. (1986) 138:872-879; G. P. Samokhin et al., FEBS Lett. (1983) 154:257-261; V. R. Muzykantov et al., J Immunol Methods. (1993) 158:183-190; M. D. Smirnov et al., Biochem Biophys Res Commun. (1983) 116:99-105; V. R. Muzykantov et al., FEBS Lett. (1985) 182:62-66; M. Magnaniet al., Biotechnol Appl Biochem. (994) 20(Pt 3):335-345; V. R. Muzykantov et al., Anal Biochem. (1994) 223:142-148; and H. Cowley et al., Transfusion (1999) 39:163-168, content of all of which is incorporated herein by reference.

Conjugation of highly hydrophilic polyethylene glycol (PEG) with the chain length in the range MW 3-10 kD has evolved as a universal “stealth” technology, prolonging circulation and masking from defense systems in the body of liposomes, nanoparticles, polymer nanocarriers, proteins, other drug carriers, and drugs themselves. Accordingly, the red blood cells described herein can by PEGylated. Without wishing to be bound by a theory, PEG-coated RBC are less effectively opsonized, taken up by phagocytes and recognized by Ries to RBC antigens. Methods of coupling PEG to RBCs are well known in the art and described, for example, in A. J. Bradley et al., Transfusion 41 (2001) pp: 1225-1233; D. Sabolovic et al., Electrophoresis 21 (2000) pp: 301-306; P. Nacharaju et al., Transfusion 45 (2005) pp: 374-383; P. Nacharaju et al. Artif Cells Blood Substit Immobil Biotechnol 35 (2007) pp: 107-118; H. A. Chunget al., J Biomed Mater Res A 70 (2004) pp 179-185; M. D. Scott et al., Proc Natl Acad Sci USA 94 (1997) pp: 7566-7571; J. K. Leach, A. Hinman and E. A. O'Rear, Biomed Sci Instrum 38 (2002) pp: 333-338; and S. Hashemi-Najafabadi et al., Bioconjug Chem 17 (2006) pp: 1288-1293, content of all of which is incorporated herein by reference. One can also modify RBCs by Pluronic, a tri-block copolymer combining two PEG chains at the ends of a less hydrophilic moiety as described in J. K. Armstrong et al., Biorheology 38 (2001) pp: 239-247, content of which is incorporated herein by reference.

In some embodiments, a RBC comprises at least one therapeutic agent and at least one imaging or contrast agent. This can be useful for simultaneous delivery of a therapeutic agen and an imaging or contrast agent for theranostic.

### Microcapsules

A compound of interest, e.g., a therapeutic agent and/or an imaging agent, can also be encapsulated in a microcapsule for delivery to a stenosis site. Accordingly, in one aspect, the invention provides a method for treating or imaging a stenosis, a stenotic lesion, a blood clot, an obstructive lesion, and/or an internal hemorrhage in a subject, the method comprising administering to a subject in need thereof a microcapsule, wherein the microcapsule comprises a therapeutic agent and/or an imaging agent. Without wishing to be bound by a theory, the microcapsule breaks apart under the elevated shear stress at the elevated shear stress at the stenosis site and releases the compound of interest (e.g., a therapeutic agent or an imaging agent).

As used herein, the term “microcapsule” means a spheroid, cube, polyhedron, prism, cylinder, rod, disc, or other geometric or irregular shape structure ranging in size from on the order of about 1 micron to about 5,000 microns composed of a distinct polymer shell, which serves as a wall-forming material, surrounding encapsulated media, e.g., a compound of interest, located within the shell. This term is distinct from microspheres, which consist of spherical homogeneous granules of a compound of interest dispersed in a polymer and are, in strict sense, spherically empty particles.

A microcapsule can be a single-layer microcapsule or a multi-layer microcapsule. As used herein the term “single-layer microcapsule” refers to a microcapsule consisting of a single polymeric shell and the encapsulated compound located within the shell in the center of the microcapsule. The term “multi-layer microcapsule” refers to a microcapsule consisting of an inner core microcapsule and one or more outer polymeric shells. The term “double-layer microcapsule” refers to a microcapsule consisting of the inner core microcapsule coated with a second polymeric shell. In the course of the microencapsulation, the core microcapsules are introduced to the polymer-plasticizer solution or polymer-mineral dispersion, and promote the formation of “embryo” shells, which are converted to a structured solid shell of double-layer microcapsules.

As used herein, the term “inner core microcapsule” refers to a single-layer microcapsule as defined above when within a double-layer or multi-layer microcapsule.

The term “wall-forming polymer” typically refers to a polymer or a combination of two or more different polymers as defined herein, which form a component of the external wall or layer or shell of the microcapsules. In some embodiments, the wall-forming polymer is a biocompatible polymer.

In some embodiments, the wall-forming polymer is a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade name Pluronic or Pluronics. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic tradename, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In some embodiment, the poloxamer is Pluronic F127.

As used herein, the term “polymer shell” refers to a polymer layer containing the wall-forming polymer and, optionally, further components such as a plasticizer and/or a mineral.

Numerous techniques for forming microcapsules are available depending on the nature of the encapsulated substance and on the type of wall-forming polymer used. A widely used method for encapsulation of water insoluble substances such as some vitamins, drugs and oils within water insoluble polymers is the solvent removal method. Generally in such a process the desired wall-forming polymer is dissolved in a suitable organic solvent. This action is followed by addition of the desired compound to be encapsulated. This compound is either dissolved or dispersed in the organic solvent. The resulting organic solution or dispersion is dispersed in an aqueous phase to obtain an oil-in-water emulsion where oily microparticles are dispersed in the aqueous phase. Upon complete removal of the solvent from the microparticles, the microcapsules are formed. A basic prerequisite for this process is the use of a solvent that is able to efficiently dissolve the compound to be encapsulated as well as the wall-forming material. This solvent has to be only partially soluble in water, giving rise to emulsion of an organic phase in a continuous water phase. Chlorinated solvents such as dichloromethane and chloroform as well as glycols or their mixtures with other solvents have been widely used since they facilitate the microencapsulation process.

Without limitations, solvent can be removed by vacuum distillation, evaporation, or extraction with water. Exemplary methods of solvent removal are described, for example, in U.S. Pat. No. 4,384,975 and No. 3,891,570, content of all of which is incorporated herein.

Methods of forming microcapsules are described, for example, in U.S. Pat. No. 3,173,878; No. 3,460,972; No. 3,516,941; No. 4,089,802; No. 4,093,556; No. 4,105,823; No. 4,140,516; No. 4,157,983; No. 4,219,604; No. 4,219,631; No. 4,221,710; No. 4,272,282; No. 4,534,783; No. 4,557,755; No. 4,574,110; No. 4,601,863; No. 4,711,749; No. 4,753,759; No. 4,898,696; No. 4,936,916; No. 4,956,129; No. 4,957,666; No. 5,011,634; No. 5,061,410; No. 5,160,529; No. 5,204,185; No. 5,236,782; No. 5,401,577; No. 5,529,877; No. 5,603,986; No. 5,650,173; No. 5,654,008; No. 5,733,561; No. 5,837,653; No. 5,861,360; No. 5,869,424; No. 6,099,864; No. 6,197,789; No. 6,248,364; No. 6,251,920; No. 6,270,836; No. 6,524,763; No. 6,534,091; No. 6,733,790; No. 6,818,296; No. 6,951,836; No. 6,969,530; No. 6,974,592; No. 7,041,277; No. 7,736,695; No. 7,803,422; No. 7,833,640; and No. 7,897,555, and U.S. Pat. Pub. No. 2003/0118822; No. 2004/0115280; No. 2004/0170693; No. 2006/0040844; No. 2007/0042184; No. 2006/0256423; No. 2009/0289216; and No. 2010/0009893, content of all of which is incorporated herein by reference. Methods of preparing multi-wall microspheres are described, for example, in U.S. No. 3,429,827; No. 4,861,627; No. 5,795,570; No. 5,985,354; No. 6,511,749; and No. 6,528,035; and U.S. Pat. App. Pub. No. 2003/0222378, content of all of which is incorporated herein by reference.

The shear stress under which a microcapsule described herein can break apart is 5 to 3000 dyn/cm². In some embodiments, the shear stress under which a microcapsule described herein breaks apart is ≧5 dyn/cm², ≧6 dyn/cm², ≧7 dyn/cm², ≧8 dyn/cm², ≧9 dyn/cm², ≧10 dyn/cm², ≧11 dyn/cm², ≧12 dyn/cm², ≧13 dyn/cm², ≧14 dyn/cm², ≧15 dyn/cm², or ≧0 dyn/cm².

As used herein, “breaking apart” refers to breaking of the polymeric shell of the microcapsule into smaller pieces. It is to be understood that complete breakup of the polymeric shell is not required. Accordingly, in some embodiments, a microcapsule can break apart such that at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, or 100% (i.e. complete breakup) of the polymeric shell is broken into smaller pieces under shear stress conditions (e.g., a stenosis site shear stress) as compared to a control shear condition (e.g., normal blood vessel shear stress).

Under elevated shear stress, the rate of release of an encapsulated compound from the microcapsule is at least 10%, at least 20%, at least 30%, at least 40%, at least 50%, at least 60%, at least 70%, at least 80%, at least 90%, at least 1-fold, at least 2-fold, at least 5-fold, at least 10-fold, at least 20-fold, at least 30-fold, at least 40-fold, at least 50-fold, or at least 100-fold or higher, relative to release under non-elevated shear stress (i.e., normal blood vessel shear stress).

In some embodiments, the amount of an encapsulated compound released from the microcapsule is at least 10%, at least 20%, at least 30%, at least 40%, at least 50%, at least 60%, at least 70%, at least 80%, at least 90%, at least 1-fold, at least 2-fold, at least 5-fold, at least 10-fold, at least 20-fold, at least 30-fold, at least 40-fold, at least 50-fold, or at least 100-fold or higher under shear stress conditions (e.g., a stenosis site shear stress) as compared to a control shear condition (e.g., normal blood vessel shear stress).

Exemplary microcapsules amenable to the present invention include those described, for example, in U.S. Pat. No. 3,173,878; No. 3,429,827; No. 3,460,972; No. 3,516,941; No. 4,089,802; No. 4,093,556; No. 4,105,823; No. 4,140,516; No. 4,157,983; No. 4,219,604; No. 4,219,631; No. 4,221,710; No. 4,272,282; No. 4,534,783; No. 4,557,755; No. 4,574,110; No. 4,601,863; No. 4,711,749; No. 4,753,759; No. 4,861,627; No. 4,898,696; No. 4,936,916; No. 4,956,129; No. 4,957,666; No. 5,011,634; No. 5,061,410; No. 5,160,529; No. 5,204,185; No. 5,236,782; No. 5,401,577; No. 5,529,877; No. 5,603,986; No. 5,650,173; No. 5,654,008; No. 5,733,561; No. 5,795,570; No. 5,837,653; No. 5,861,360; No. 5,869,424; No. 5,985,354; No. 6,099,864; No. 6,197,789; No. 6,248,364; No. 6,251,920; No. 6,270,836; 6,511,749; No. 6,524,763; No. 6,528,035; No. 6,534,091; No. 6,733,790; No. 6,818,296; No. 6,951,836; No. 6,969,530; No. 6,974,592; No. 7,041,277; No. 7,736,695; No. 7,803,422; No. 7,833,640; and No. 7,897,555, and U.S. Pat. Pub. No. 2003/0118822; No. 2003/0222378No. 2004/0115280; No. 2004/0170693; No. 2006/0040844; No. 2007/0042184; No. 2006/0256423; No. 2009/0289216; and No. 2010/0009893, content of all of which is incorporated herein by reference.

In some embodiments, a microcapsule comprises at least one therapeutic agent and at least one imaging or contrast agent. This can be useful for simultaneous delivery of a therapeutic agen and an imaging or contrast agent for theranostic.

### Compounds of Interest

A wide variety of compounds can be associated with aggregates, red blood cells and microcapsules. Accordingly, without limitation, the compound of interest can be selected from the group consisting of small or large organic or inorganic molecules, carbon-based molecules (e.g., nanotubes, fullerenes, buckeyballs, and the like), metals (e.g., alkali metals, e.g., lithium, sodium, potassium rubidium, caesium, and francium; alkaline earth metals, e.g., beryllium, magnesium, calcium strontium, barium, and radium; transition metals, e.g., zinc, molybdenum, cadmium scandium, titanium, vanadium chromium, manganese, iron cobalt, nickel, copper yttrium, zirconium, niobium technetium, ruthenium, rhodium palladium, silver, hafnium tantalum, tungsten, rhenium osmium, iridium, platinum gold, mercury, rutherfordium, dubnium, seaborgium, bohrium, hassium, and copernicium; post-transition metals, e.g., aluminium, gallium, indium, tin thallium, lead, bismuth; lanthanides, e.g., lanthanum, cerium, praseodymium neodymium, promethium, samarium europium, gadolinium, terbium dysprosium, holmium, erbium thulium, ytterbium, and lutetium; actinides (e.g., actinium, thorium, protactinium uranium, neptunium, plutonium americium, curium, berkelium californium, einsteinium, fermium mendelevium, nobelium, and lawrencium; meitnerium; darmstadtium; roentgenium ununtrium; flerovium ununpentium; livermorium germanium; arsenic; antimony; polonium; and astatine), metal oxides (e.g., titanium dioxide (TiO₂), iron oxides (e.g., Fe₃O₄, Fe₂O₃, and the like), aluminum oxide, antimony tetraoxide, antimony oxide, arsenous oxide, beryllium oxide, bismuth oxide, cadmium oxide, chromic oxide, cobaltic oxide, gallium dioxide, germanium dioxide, hafnium oxide, indium oxide, lead oxide, magnesium oxide, mercuric oxide, molybdenum trioxide, nickel monoxide, niobium pentaoxide, scandium oxide, selenium dioxide, silicon dioxide, silver oxide, tantalum pentaoxide, tellurium dioxide, thallic oxide, thorium oxide, stannic oxide, tungsten trioxide, uranium oxide, vanadium pentoxide, ytrrium oxide, zinc oxide, zirconium dioxide, ceric oxide, dysprosium oxide, erbium oxide, europium oxide, gadolinium oxide, holmium oxide, lanthanum sesquioxide, lutetium oxide, neodymium oxide, samarium oxide, terbium peroxide, thulium oxide, ytterbium oxide, PuO₂, and the like), nanoparticles (e.g., metal nanoparticles, inorganic nanoparticles, gold nanoparticles, silica nanoparticles, calcium carbonate nanoparticles, and the like), imaging agents, contrast agents, monosaccharides, disaccharides, trisaccharides, oligosaccharides, polysaccharides, amino acids, biological macromolecules, e.g., peptides, proteins, peptide analogs and derivatives thereof, peptidomimetics, nucleic acids, nucleic acid analogs and derivatives, polynucleotides, oligonucleotides, enzymes, antibodies and portions and fragments thereof, monoclonal antibodies and portions and fragments thereof, polyclonal antibodies and portions and fragments thereof, an extract made from biological materials such as bacteria, plants, fungi, or animal cells or tissues, naturally occurring or synthetic compositions, particulates, non-aggregating nanoparticles, or any combinations thereof. The compound can be hydrophobic, hydrophilic, or amphiphilic.

In some embodiments, the molecule is therapeutic agent and is a monoclonal antibody or fragment thereof or a polyclonal antibody or fragment thereof.

In some embodiments, the molecule is diagnostic agent and is a monoclonal antibody or fragment thereof or a polyclonal antibody or fragment thereof.

In some embodiments, the molecule is a targeting ligand and is a monoclonal antibody or fragment thereof or a polyclonal antibody or fragment thereof.

As used herein, the term “particulate” refers to a particle, powder, flake, etc., that inherently exists in a relatively small form and may be formed by, for example, grinding, shredding, fragmenting, pulverizing, atomizing, or otherwise subdividing a larger form of the material into a relatively small form.

As used herein, the term “non-aggregating nanoparticle” refers to nanoparticles that do not aggregate under the conditions for aggregation described herein.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is highly preferred that a small molecule have a molecular mass equal to or less than 700 Daltons.

In one embodiment, the compound is a peptide or a protein. As used herein, the term “peptide” is used in its broadest sense to refer to compounds containing two or more amino acids, amino acid equivalents or other non-amino groups joined to each other by peptide bonds or modified peptide bonds. Peptide equivalents can differ from conventional peptides by the replacement of one or more amino acids with related organic acids (such as PABA), amino acids or the like or the substitution or modification of side chains or functional groups. A peptide can be of any size so long; however, in some embodiments, peptides having twenty or fewer total amino acids are preferred. Additionally, the peptide can be linear or cyclic. Peptide sequences specifically recited herein are written with the amino terminus on the left and the carboxy terminus on the right.

In addition, the term “peptide” broadly includes proteins, which generally are polypeptides. As used herein, the term “protein” is used to describe proteins as well as fragments thereof. Thus, any chain of amino acids that exhibits a three dimensional structure is included in the term “protein”, and protein fragments are accordingly embraced.

A peptidomimetic is a molecule capable of folding into a defined three-dimensional structure similar to a natural peptide As used herein, the term “nucleic acid” refers to a polymers (polynucleotides) or oligomers (oligonucleotides) of nucleotide or nucleoside monomers consisting of naturally occurring bases, sugars and intersugar linkages. The term “nucleic acid” also includes polymers or oligomers comprising non-naturally occurring monomers, or portions thereof, which function similarly. Such modified or substituted nucleic acids are often preferred over native forms because of properties such as, for example, enhanced cellular uptake and increased stability in the presence of nucleases.

A nucleic acid can be single-stranded or double-stranded. A single-stranded nucleic acid can have double-stranded regions and a double-stranded nucleic acid can have single-stranded regions. Exemplary nucleic acids include, but are not limited to structural genes, genes including control and termination regions, self-replicating systems such as viral or plasmid DNA, modified RNAs, single-stranded and double-stranded siRNAs and other RNA interference reagents (RNAi agents or iRNA agents), short-hairpin RNAs (shRNA), hairpin DNAs, self-assemblying RNAs or DNAs, antisense oligonucleotides, ribozymes, microRNAs, microRNA mimics, aptamers, antimirs, antagomirs, triplex-forming oligonucleotides, RNA activators, immuno-stimulatory oligonucleotides, and decoy oligonucleotides. The nucleic acid can comprise one or more nucleic acid modifications known in the art.

In some embodiments of this and other aspects of the invention described herein, the compound is biologically active or has biological activity.

As used herein, the term “biological activity” or “bioactivity” refers to the ability of a compound to affect a biological sample. Biological activity can include, without limitation, elicitation of an adhesive, polymerization, stimulatory, inhibitory, regulatory, toxic or lethal response in a biological assay at the molecular, cellular, tissue or organ levels. For example, a biological activity can refer to the ability of a compound to exhibit or modulate the effect/activity of an enzyme, block a receptor, stimulate a receptor, modulate the expression level of one or more genes, modulate cell proliferation, modulate cell division, modulate cell morphology, or any combination thereof. In some instances, a biological activity can refer to the ability of a compound to produce a toxic effect in a biological sample, or it can refer to an ability to chemical modify a target molecule or cell. The biological activity can be inside a cell or outside of a cell.

The aggregate or the nanoparticle constituent of the aggregate can be internalized into a cell of interest with the biological activity occurring inside the cell after internalization. Accordingly, in some embodiments, the aggregate or nanoparticle constituent of the aggregate are biologically active following internalization into a cell.

In some embodiments of this and other aspects of the invention, the compound is a therapeutic agent. As used herein, the term “therapeutic agent” refers to a biological or chemical agent used for treatment, curing, mitigating, or preventing deleterious conditions in a subject. The term “therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. The terms “therapeutic agent” and “pharmaceutically active agent” are used interchangeably herein.

The therapeutic agent is selected according to the treatment objective and biological action desired. General classes of therapeutic agents include anti-microbial agents such as adrenergic agents, antibiotic agents or antibacterial agents, antiviral agents, anthelmintic agents, anti-inflammatory agents, antineoplastic agents, antioxidant agents, biological reaction inhibitors, botulinum toxin agents, chemotherapy agents, diagnostic agents, gene therapy agents, hormonal agents, mucolytic agents, radioprotective agents, radioactive agents including brachytherapy materials, tissue growth inhibitors, tissue growth enhancers, vasoactive agents, thrombolytic agents (i.e., clot busting agents), inducers of blood coagulation, and inhibitors of RBC aggregation in Sickle Cell Disease.

The therapeutic agent can be selected from any class suitable for the therapeutic objective. For example, if the objective is treating a disease or condition associated stenosis, the therapeutic agent may include antithrombotic or thrombolytic agent or fibrinolytic agents. By way of further example, if the desired treatment objective is treatment of cancer, the therapeutic agent may include radioactive material in the form of radioactive seeds providing radiation treatment directly into the tumor or close to it. Further, the therapeutic agent may be selected or arranged to provide therapeutic activity over a period of time.

Exemplary pharmaceutically active compound include, but are not limited to, those found in Harrison's Principles of Internal Medicine, 13^{th} Edition, Eds. T. R. Harrison McGraw-Hill N.Y., NY; Physicians' Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

In some embodiments of this and other aspects of the invention, the therapeutic agent is an antithrombotic or thrombolytic agent or fibrinolytic agent selected from the group consisting of anticoagulants, anticoagulant antagonists, antiplatelet agents, thrombolytic agents, thrombolytic agent antagonists, and any combinations thereof.

In some embodiments of this and other aspects of the invention, the therapeutic agent is thrombogenic agent selected from the group consisting of thrombolytic agent antagonists, anticoagulant antagonists, pro-coagulant enzymes, pro-coagulant proteins, and any combinations thereof. Some exemplary thrombogenic agents include, but are not limited to, protamines, vitamin K1, amiocaproic acid (amicar), tranexamic acid (amstat), anagrelide, argatroban, cilstazol, daltroban, defibrotide, enoxaparin, fraxiparine, indobufen, lamoparan, ozagrel, picotamide, plafibride, tedelparin, ticlopidine, triflusal, collagen, and collagen-coated particles.

In some embodiments of this and other aspects of the invention, the therapeutic agent is a thrombolytic agent. As used herein, the term “thrombolytic agent” refers to any agent capable of inducing reperfusion by dissolving, dislodging or otherwise breaking up a clot, e.g., by either dissolving a fibrin-platelet clot, or inhibiting the formation of such a clot. Reperfusion occurs when the clot is dissolved and blood flow is restored. Exemplary thrombolytic agents include, but are not limited to, plasmin, tissue-type plasminogen activator (t-PA), streptokinase (SK), prourokinase, urokinase (uPA), alteplase (also known as Activase®, Genentech, Inc.), reteplase (also known as r-PA or Retavase®, Centocor, Inc.), tenecteplase (also known as TNK™, Genentech, Inc.), Streptase® (AstraZeneca, LP), lanoteplase (Bristol-Myers Squibb Company), monteplase (Eisai Company, Ltd.), saruplase (also known as r-scu-PA and Rescupase™, Grunenthal GmbH, Corp.), staphylokinase, and anisoylated plasminogen-streptokinase activator complex (also known as APSAC, Anistreplase and Eminase®, SmithKline Beecham Corp.). Thrombolytic agents also include other genetically engineered plasminogen activators. The invention can additionally employ hybrids, physiologically active fragments or mutant forms of the above thrombolytic agents. The term “tissue-type plasminogen activator” as used herein is intended to include such hybrids, fragments and mutants, as well as both naturally derived and recombinantly derived tissue-type plasminogen activator.

Other thromolytic agents for use in the invention include, but are not limited to, A-74187; ABC-48; adenosine for cardioprotection, King Pharma R&D; alfimeprase; alpha2-antiplasmin replacement therapy, Bayer; alteplase; amediplase; ANX-188; argatroban; arimoclomol; arundic acid (injectable formulation), Ono; asaruplase; ATH (thromboembolism/thrombosis), Inflazyme; atopaxar; BGC-728; bivalirudin; BLX-155; ciprostene; clazosentan; clomethiazole; clopidogrel; conestat alfa; CPC-211; desirudin; desmoteplase; DLBS-1033; DP-b99; DX-9065a; ebselen; echistatin, Merck & Co; edoxaban; efegatran; eptifibatide; erlizumab; EU-C-002; FK-419; fondaparinux sodium; H-290/51; hirudin-based thrombin inhibitors, BMS; HRC-102; ICI-192605; inogatran; lamifiban; lanoteplase; lumbrokinase; LY-210825; M5, Thrombolytic Science; melagatran; monteplase; MRX-820; nasaruplase; nicaraven; non-thrombolytic proteins, Genzyme; ocriplasmin (injected, stroke), Thrombogenics; ocriplasmin (ophthalmic), ThromboGenics/Alcon; ONO-2231; paclitaxel (lipid-based complex), MediGene; PB-007; PEGylated recombinant staphylokinase variant, ThromboGenics/Bharat Biotech; pexelizumab; Pro-UK; pro-urokinase, Erbamont; recombinant c1 esterase inhibitor (cardiovascular diseases), TSI; recombinant plasmin (vascular occlusion/ocular disease), Talecris Biotherapeutics/Bausch & Lomb; reteplase; saruplase; scuPA/suPAR (MI, stroke), Thrombotech; SM-20302; staplabin, Tokyo Noko; STC-387; SUPG-032; TA-993; TAFI inhibitors (thrombosis/myocardial infarction/stroke), Berlex; tenecteplase; TH-9229; THR-174; THR-18; tPA-HP; tridegin; troplasminogen alfa; urokinase; YM-254890; YM-337; YSPSL; and the like.

The term “anticoagulant” is meant to refer to any agent capable of prolonging the prothrombin and partial thromboplastin time tests and reducing the levels of prothrombin and factors VII, IX and X. Anticoagulants typically include coumarin derivatives and heparin as well as aspirin, which may also be referred to as an antiplatelet agent.

In some embodiments of this and other aspects of the invention, the therapeutic agent is a pro-angiogenesis agent. As used herein, pro-angiogenic agents are molecules or compounds that promote the establishment or maintenance of the vasculature. Such agents include agents for treating cardiovascular disorders, including heart attacks, strokes, and peripheral vascular disease.

In some embodiments, the therapeutic agent is an anti-adhesive agent, an anti-platelet agent, or an anti-polymerization agent.

In some embodiments of this and other aspects of the invention, the pharmaceutically active agent include those agents known in the art for treatment of inflammation or inflammation associated disorders, or infections. Exemplary anti-inflammatory agents include, but are not limited to, non-steroidal anti-inflammatory drugs (NSAIDs—such as aspirin, ibuprofen, or naproxen), coricosteroids (such as presnisone), anti-malarial medication (such as hydrochloroquine), methotrexrate, sulfasalazine, leflunomide, anti-TNF medications, cyclophosphamise, mycophenolate, dexamethasone, rosiglitazone, prednisolone, corticosterone, budesonide, estrogen, estrodiol, fenfibrate, provastatin, simvastatin, proglitazone, acetylsalicylic acid, mycophenolic acid, mesalamine, hydroxyurea, and analogs, derivatives, prodrugs, and pharmaceutically acceptable salts thereof.

In some embodiments of this and other aspects of the invention, the pharmaceutically active agent is a vasodilator. A vasodilator can be selected from the group consisting of alpha-adrenoceptor antagonists (alpha-blockers), angiotensin converting enzyme (ACE) inhibitors, angiotensin receptor blockers (ARBs), beta2-adrenoceptor agonists (β2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, renin inhibitors, and any combinations thereof. Exemplary vasodilator include, but are not limited to, prazosin, terazosin, doxazosin, trimazosin, phentolamine, phenoxybenzamine, benazepril, captopril, enalapril, fosinopril, lisinopril, moexipril, quinapril, ramipril, candesartan, eprosartan, irbesartan, losartan, olmesartan, telmisartan, valsartan, Epinephrine, Norepinephrine, Dopamine, Dobutamine, Isoproterenol, amlodipine, felodipine, isradipine, nicardipine, nifedipine, nimodipine, nitrendipine, clonidine, guanabenz, guanfacine, α-methyldopa, hydralazine, Bosentan, trimethaphan camsylate, isosorbide dinitrate, isosorbide mononitrate, nitroglycerin, erythrityl tetranitrate, pentaerythritol tetranitrate, sodium nitroprusside, milrinone, inamrinone (formerly amrinone), cilostazol, sildenafil, tadalafil, minoxidil, aliskiren, and analogs, derivatives, prodrugs, and pharmaceutically acceptable salts thereof.

In some embodiments of this and other aspects of the invention, the pharmaceutically active agent is a vasoconstrictor. As used herein, the term “vasoconstrictor” refers to compounds or molecules that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness. Other descriptive names of the vasoconstrictor group include vasoactive agonists, vasopressor agents and vasoconstrictor drugs. Certain vasoconstrictors act on specific receptors, such as vasopressin receptors or adrenoreceptors. Exemplary vasoconstrictors include, but are not limited to, alpha-adrenoreceptor agonists, chatecolamines, vasopressin, vasopressin receptor modualors, calcium channel agonists, and other endogenous or exogenous vasoconstrictors.

In some embodiments, the vasoconstrictor is selected from the group consisting of aluminum sulfate, amidephrine, amphetamines, angiotensin, antihistamines, argipressin, bismuth subgallate, cafaminol, caffeine, catecholamines, cyclopentamine, deoxyepinephrine, dopamine, ephedrine, epinephrine, felypressin, indanazoline, isoproterenol, lisergic acid diethylamine, lypressin (LVP), lysergic acid, mephedrone, methoxamine, methylphenidate, metizoline, metraminol, midodrine, naphazoline, nordefrin, norepinephrine, octodrine, ornipressin, oxymethazoline, phenylefhanolamine, phenylephrine, phenylisopropylamines, phenylpropanolamine, phenypressin, propylhexedrine, pseudoephedrine, psilocybin, tetrahydralazine, tetrahydrozoline, tetrahydrozoline hydrochloride, tetrahydrozoline hydrochloride with zinc sulfate, tramazoline, tuaminoheptane, tymazoline, vasopressin, vasotocin, xylometazoline, zinc oxide, and the like.

In some embodiments, the vasoactive agent is a substance derived or extracted from a herbal source, selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), cypress and salts, isomers, analogs and derivatives thereof.

In some embodiments of this and other aspects of the invention, the pharmaceutically active agent is an anti-neoplastic, anti-proliferative, and/or anti-miotic agent. Exemplary anti-neoplastic/anti-proliferative/anti-miotic agents include, but are not limited to, paclitaxel (taxol), 5-fluorouracil, doxorubicin, daunorubicin, cyclosporine, cisplatin, vinblastine, vincristine, epothilones, methotrexate, azathioprine, adriamycin and mutamycin; endostatin, angiostatin and thymidine kinase inhibitors, cladribine, trapidil, halofuginone, plasmin, and analogs, derivatives, prodrugs, and pharmaceutically acceptable salts thereof.

In some embodiments, the pharmaceutically active agent has a very short half-life in blood or serum. For example, the pharmaceutically active agent has a half-life of 1 minute, 2 minutes, 3 minutes, 4 minutes, 5 minutes, 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 8 hours, 9 hours, 10 hours, 11 hours, or 12 hours or less, in blood or serum. These short lifetime agents can have a local effect.

In some embodiments of this and other aspects of the invention, the therapeutic agent is selected from the group consisting of aspirin, wafarin (coumadin), acenocoumarol, ancrod, anisindione, bromindione, clorindione, coumetarol, cyclocumarol, dextran, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol, dipyridamole (persantin), sulfinpyranone (anturane), ticlopidine (ticlid), tissue plasminogen activator (activase), plasmin, pro-urokinase, urokinase (abbokinase) streptokinase (streptase), and anistreplase/APSAC (eminase), and analogs, derivatives, prodrugs, and pharmaceutically acceptable salts thereof.

In some embodiments of this and other aspects of the invention, the pharmaceutically active agent is an agent for treatment of arterial occlusive disease. Exemplary agents for treatment of arterial occlusive disease include, but are not limited to 11beta-hydroxysteroid dehydrogenase-1 (HSD1) inhibitors, Merck & Co; 15-LO inhibitors, Bristol-Myers Squibb; 18C3 (anti-IL-1 alpha true human antibody), XBiotech; 2,3-dioxoindoline, Qingdao University; 2164U90; 2-5A antisense inhibitors (RSV), Ridgeway; 2NTX-99; 3,4-di(OH)-hydrocinnamante derivatives (oral, hyperlipidemia/atherosclerosis), KRIBB; 447C88; 568859; 99mTc-anti-ED-B; 99mTc-AP(4)A; 99mTc-P215; A-104029; A-203719; A-206377; A-207508; A-76341; A-87049; ABCA1/ApoA1 (atherosclerosis), Gilead Palo Alto; ABT-306552; AC-3056; ACAT inhibitor (atherosclerosis), Kyoto; ACAT inhibitors (atherosclerosis), Takeda; ACAT inhibitors, Azwell; ACAT inhibitors, Kyowa Hakko Kogyo; ACAT inhibitors, Schering-Plough; acetylsalicylic acid+simvastatin (atherosclerosis), HanAll Biopharma; acifran; acitemate; ACP-501; acyl-CoA cholesterol acyltransferase inhibitor/diacylglycerol acyltransferase inhibitor/apolipoprotein-A1 stimulator (atherosclerosis), Kyoto; Ad2/FasL/p35 gene therapy, Genzyme Corp; Ad5-NOS gene therapy, Schering AG; adiponectin mimetics (oral, type 2 diabetes/atherosclerosis/muscle metabolic diseases), Rigel Pharmaceuticals; Adpk7; ADR-7; AFP-07; AFS-98; AG-1295; AGI-3; AGI-H1; AGI-H-15; AHRO-001; AIM-501; AJ-814; AKB-9778; AL-0671; ALD-301; alendronate (iv liposomal, restenosis), BIOrest; alfimeprase; AlleKine; alpha-v/beta-3 antagonists, J&J PRD; alprostadil (lipid microsphere formulation), Taisho/Mitsubishi Tanabe; aminoguanidine, INSERM; amyloid modulators (type 2 diabetes/atherosclerosis), Crossbeta Biosciences; ANG-1170; anti-alpha-v/beta-3 mAb, SmithKline Beecham; anticholesterolemics, Pfizer; anti-midkine antibodies (cancer/RA/MS), Cellmid; antioxidant-containing curcumin analogs (cancer/restenosis), Ohio State University; antisense oligonucleotides (restenosis), Genta/CVT/Genta Jago; APA-01+atorvastatin (atherosclerosis), Phosphagenics; apical sodium-dependent bile acid transporter inhibitors (atherosclerosis), Sankyo; ApoA1 upregulating agents (atherosclerosis), GSK; apolipoprotein AI analogs, Fournier; Apovasc; APP-018; ARI-1778; arNOX inhibitors (oral, atherogenesis), NOX Technologies; arteriosclerosis therapy (antisense oligonucleotide), Shinshu; arteriosclerosis therapy, Daiichi; AS-013; aspalatone; Astenose; AT-1015; ataciguat; ATH-03; Atherocort; atherogenesis preventative therapy (atherosclerosis), RxBio; atherosclerosis therapy, Allelix/Fournier; atherosclerosis therapy, Aventis Gencell/INSERM; atherosclerosis therapy, Cue Biotech; atherosclerosis therapy, Millennium/Lilly; atherosclerosis therapy, Rhone-Poulenc Rorer; atherosclerosis/rheumatoid arthritis agents (sustained release/CTP), PROLOR Biotech; atherosclerotic plaque therapeutics, Zydus-Cadila; ATI-5261; atorvastatin+acetylsalicylic acid (atherosclerosis), HanAll Biopharma; atreleuton; ATZ-1993; autologous CD133+hematopoietic stem cells (peripheral artery disease), University of Wisconsin-Madison; autologous CD34+stem cell therapy (peripheral artery disease), University of Debrecen; autologous endothelial progenitor cell therapy (ischemia), IBRI; autologous fat-derived stem cell therapy, RNL Bio; avasimibe; AVE-9488; AVEX-1; AVI-4126 (injectable formulation, cancer/kidney disease), AVI; AVI-4126 (oral formulation), AVI; AVI-5126; AVT-03; AVT-06; AX-200; axitirome; AY-9944; azalanstat; AZM-008; barixibat; BAY-1006451; BAY-38-1315; BAY-60-5521; BB-476; beperminogene perplasmid; beraprost sodium; bervastatin; bFGF inhibitors, Genzyme Mol Oncology; BI-204; BIBB-515; BIBX-79; Biglycan; bile acid inhibitors, Hoechst; bindarit; Bio-Flow; Bioral ApoA1; Biostent; BL-3050; BLX-155; BMS-180431; BMS-183743; BMS-188494; BMS-192951; BMS-197636; BMS-200150; BMS-212122; BMS-582949; BMS-753951; BMS-779788; BO-653; BP-42, Toyama; burixafor; c-1602; c-2447; c-8834; canakinumab; candesartan; Capiscint; carbon monoxide (inhaled, organ transplan/cystic fibrosis/restenosis/liver failure), Ikaria; cardiovascular disease therapeutic, Lexicon/Abgenix; Carfostin; carvastatin; cathepsin S inhibitors, GlaxoSmithKline; cathepsin S inhibitors, Molecumetics/Choongwae; CCR2 antagonist (atherosclerosis), GlaxoSmithKline; CCR2 antagonists, Incyte/Pfizer; CCR2 antagonists, Millennium/Pfizer/Kyowa; CCX-140; CCX-915; CD34+stem cell therapy (myocardial ischemia/peripheral arterial occlusive disease), Northwestern University/Baxter; CD36 receptor-specific hexarelin analogs, Ardana; cdk inhibitors (restenosis), Gilead Palo Alto; CDK inhibitors, Institut Curie; CDP-860; cerivastatin; CETi-1; CETP inhibitor, Sandoz; CETP inhibitors (atherosclerosis), Merck & Co; CETP inhibitors (dyslipidemia), Bayer/Merck; CETP inhibitors, Pfizer; CETP inhibitors, Schering-Plough; CGP-43371; CGS-23425; CGS-24565; CGS-26303; CGS-26393; chemotaxin inhibitor, CV Therapeutics; chimeraplast; CHIR-11509; chitosan ester (atherosclerosis), Ocean University of China; Cholazol; cholesterol absorption inhibitors, Schering-Plough; cholesteryl ester transfer protein inhibitors (hyperlipidemia/atherosclerosis), Lilly; chymase inhibitors, Dainippon Sumitomo; CI-101; CI-976; CI-999; cilostazol; cilostazol (sustained release), Korea United Pharm; cilostazol+Ginkgo biloba extract (oral, arterial occlusive disease/stroke), SK Chemicals; ciprofibrate; CL-277082; CL-283546; CL-283796; clopidogrel+acetylsalicylic acid (oral, atherosclerosis), Dong-A; COR-2; COR-3; CP-105191; CP-113818; CP-230821; CP-340868; CP-532623; CP-800569; CP-83101; CP-88488; CPG-603; CRD-510; crilvastatin; CS-8080; CSL-111; CT-1, Channel Therapeutics; CT-2, Channel Therapeutics; CT-301/R; CT-8, Channel; CTCM-163; CVT-634; CVX-210-H; CXCR2 antagonists, Fournier Pharma; CXCR3/CCR1 antagonists, Millennium/Kyowa; CY-1748; CYC-10424; cyclodextrin derivatives, AMRAD; cytokine inhibitor, Teijin; D-11-1580; dalvastatin; darapladib; DE-112; decarestrictine D; dehydroepiandrosterone, Jenapharm; DG-041; DGAT inhibitors (atherosclerosis), AstraZeneca; dilmapimod; dipyridamole+acetylsalicylic acid (stroke), Boehringer; Dival; DMP-565; doconexent ethyl ester+icosapent ethyl ester; Docosixine; domitroban; DRF-4832; DRL-16805; DRL-17822; DuP-128 analogs, DuPont; DYB-143; dyslipidemia therapy, Bayer; E2F inhibitors (cancer), TopoTarget/InhibOx; E-5050; E-5324; EF-12; efipladib; eflucimibe; EGF fusion proteins, Ligand; eldacimibe; endometrial regenerative cells (critical limb ischemia/heart failure), Medistem; endothelial lipase antisense inhibitors (atherosclerosis), Isis; endothelin antagonist (azole), Abbott; endothelin antagonists, Abbott; EP-1242; EP3 inhibitors (peripheral arterial occlusive disease), deCODE; ESP-24218; estrogen receptor beta modulators (triazine), GlaxoSmithKline; ET-642; ETC-1001; ETC-588; ETS1 gene therapy (ischemia/myocardial infarction/angina), AnGes; ETX-6107; F-10863A; F-12509A; F-1394; F-2833; farnesoid X receptor agonists, Allergan; farnesoid X receptor antagonists, Allergan; FCP-3P1; FE-301; first-generation niacin receptor agonists (oral, atherosclerosis), Merck/Arena; fluvastatin; fosinopril; fostamatinib; FR-129169; FR-145237; FR-186054; FR-186485; frount inhibitors (inflammatory diseases/arteriosclerosis), ECl/Astellas; FY-087; gadolinium texaphyrins (imaging, atherosclerosis), Pharmacyclics; GAL T-2 inhibitors (restenosis/PKD/atherosclerosis/inflammation/AMD), Amalyte Pharmaceuticals; gantofiban; GAX-1 gene therapy, Aventis; gemcabene; gemfibrozil analogs, Novartis; gene therapy (betaARKct), Genzyme; gene therapy (cardiovascular), Somatix/Rockefeller; gene therapy (eNOS), Valentis/Ark Therapeutics; gene therapy (p16/p27), GPC Biotech; Genevx; GenStent; GERI-BP-001; glenvastatin; glutathione peroxidase mimetics (oral, atherosclerosis), Provid; glycolipid metabolites, Kitasato; glyco-S-nitrosothiols, University of Miami; Glysopep; goxalapladib; GPR25 antagonists (myocardial infarction/stroke/atherosclerosis), Omeros; GR-328713; GT-16-239; GW-2331; GX-401 program; H-290/30; halofuginone (oral, Duchenne muscular dystrophy), Halo Therapeutics; HDL cholesterol enhancers (atherosclerosis/coronary artery disease), Wyeth; HDL delipidation therapy (LSI-S955, atherosclerosis), Lipid Sciences; HDL elevating/lipid regulating agents, Pfizer/Esperion; hE-18A; heparanase inhibitors, Progen; heparin (EPT cardiovascular therapy), Inovio; HGF, Sumitomo; HL-004; HL-135; HMG-CoA inhibitors, BMS; HMG-CoA inhibitors, Pfizer; HMG-CoA reductase inhibitors, Glaxo; HR-1671; HRE-based gene therapy (cardiovascular), Aventis; hyaluronan (intravenous), SkyePharma; hypolipemic agents, Aventis/Amylin; IBT-9302; ICI-245991; icosapent ethyl ester; icrucumab; ILlaQb therapeutic vaccines (atherosclerosis), Cytos; Imidate; immuno-angioplasty, Immunomedics; immunotherapeutic vaccine (atherosclerotic plaque), Aterovax/INSERM; INC-106; NCB-3284; indole-based endothelin antagonists, Pfizer; INGN-251; iNOS lipoplex gene therapy (restenosis), Cardion; int6 gene/hypoxia-inducible factor targeting siRNA (siChimera, peripheral arterial disease), alphaGEN; integrin alpha-V/beta-3 receptor mAb (atherosclerosis), Vascular Pharmaceuticals; integrin antagonists, 3-Dimensional Pharmaceuticals; interferon beta gene therapy (electroporation/TriGrid/im, multiple sclerosis), Ichor Medical Systems; INV-400 series; INX-3280; iroxanadine; isradipine; IT-9302; ixmyelocel-T; J-104123; JTV-806; jumonji-domain-containing-3 modulators (cancer/allergy/atherosclerosis), Osaka University; K-134; K-604; KC-706; KD-025; KF-17828; KH-01500; KH-01501 series; KI-0002; KI-1004, Kereos; kininogen domain 5 peptides, DuPont; KM-011; KRN-4884; KY-331; KY-455; L-166143; L-659699; L-669262; L-731120; lacidipine; lanreotide (depot formulation), Ipsen; laropiprant+extended-release niacin+simvastatin (coronary artery disease), Merck & Co; LCAT gene therapy, NIH; Lck tyrosine kinase inhibitors, BMS; LDL gene, Genetic Therapy; LDL receptor gene therapy (restenosis), iCell; lecimibide; lercanidipine; Levulan; LF-08-0133; LF-13-0491c; lifibrol; limaprost; lipid modulators, BioCache; lipid peroxidation inhibitors, Servier; lipoprotein a inhibitors, Pfizer; liposomal prostaglandin E-1, Endovasc; LK-903; losmapimod; lovastatin; LPCN-1012; LS-3115; LT-0101; luteusin-C; LXR agonists (Alzheimers disease), Anagen Therapeutics; LXR agonists (atherosclerosis), F. Hoffmann-La Roche; LXR agonists (atherosclerosis/dyslipidemia/Alzheimer's disease), AstraZeneca; LXR agonists (dyslipidemia/atherosclerosis/diabetes), Tanabe; LXR modulators (atherosclerosis), Vitae Pharmaceuticals; LXR modulators (hypercholesterolemia/atherosclerosis), Phenex; LXR modulators (inflammation), Karo Bio/Pfizer; LY-2157299; LY-295427 analogs, Lilly; LY-674; lysosomal acid lipase, LSBC; mammalian sterile 20-like kinase 1 gene eluting stent (restenosis), Vasade; MAP kinase inhibitors (inflammation/pain/fibrosis), Allinky; marsidomine; MBX-2599; MC-031; MC-032; MC-033; MC-034; MCP-1 inhibitors, Millennium/Pfizer; MCP-1 inhibitors, Roche/Iconix; MDCO-216; MDL-28815; MDL-29311; merilimus eluting coronary stents (restenosis), Meril Life Sciences; Mesendo; MGN-2677; MIF antagonists (inflammation), Cortical; mimic HMGB-1 antibodies (restenosis/atherosclerosis), Bio3; misoprostol; MK-0736; MK-1903; MK-6213; MKC-121; MLN-1202; MMI-270; MMP-12 inhibitors (atherosclerosis), CEA; MMP-13 inhibitors (arthritis), Wyeth; MOL-376; molecularly imprinted polymers (hyperphosphatemia), Semorex; monoclonal antibody (atherosclerosis), Scotgen; motexafin lutetium; MRZ-3/124; MT1-MMP inhibitors, 3DP; MTP inhibitors, Leiden University; MTP-131; muparfostat; MV-6401; mycophenolate mofetil; myeloperoxidase inhibitors (oral/small molecule, atherosclerosis), Torrey Pines; N,N′-diacetyl-L-cystine; N-1177-iv; N-4472; naAGs (inflammation, cancer, atherosclerosis, AMD, or COPD), SelectX; NB-598; Neutralase; NFkappa B/E2F chimeric decoy oligonucleotides (inflammation), AnGes; NI-0401; nicotinic acid+lovastatin, Kos/Merck KGaA; nicotinic acid 1 receptor (GPR109A) agonists, Merck; NIK modulators, Celgene; Nimoxine; nitrosated albumin; NMDA receptor antagonists (atherosclerosis), University of Nebraska Medical Center; NO synthase modulators, CNRS; Novolimus eluting coronary stents (restenosis), Elixir Medical; NPH-4; NTE-122; NV-27; ocriplasmin (injected, stroke), Thrombogenics; olcorolimus (restenosis), Elixir Medical/Novartis; oligonucleotide (myosin IIB), Ludwig-Maximilians; oligonucleotide decoys (E2F), Fujisawa/Osaka; oligonucleotide decoys (NFkappa B, restenosis/psoriasis/atopic dermatitis/peridontal/respiratory/bone diseases), AnGes/Shionogi/Medikit/Hosokawa; oligonucleotide decoys (NFkappaB), Osaka University; OPC-35564; Org-13061; ORP-150 inducers (arteriosclerosis/ischemic heart disease/cancer/diabetes mellitus), HSP Research Institute; P-06103; P-06133; P-06139; P-0654; P-2202; P2Y12 inhibitor (oral, atherothrombosis), LG Life Sciences; P-773; P-947; paclitaxel (Vascular Magnetic Intervention technology/nanoparticle formulation, peripheral artery diseases), Vasular Magnetics; paclitaxel (Zyn-linkers delivery technology, restenosis), Zynaxis; pactimibe; PAI-1 antagonists, 3DP; pamaqueside; Pantarin; PAR1 antagonist (thrombosis/restenosis), Pierre Fabre; PAR-1 antagonists (thrombosis), Eisai; PAR-1 receptor antagonists (arteriosclerosis/vascular disease), KRICT; parogrelil; PC-mAb; PD-089244; PD-089828; PD-098063; PD-129337; PD-13201-2; PD-132301-2; PD-135022; PD-146176; PD-148817; PD-161721; PD-166285; PDE4/MMP inhibitors, Rhone-Poulenc; PDGF receptor kinase inhibitor, Yissum; PDGF receptor kinase inhibitors, AEterna Zentaris; PDGF receptor program, Millennium; PDGF TK antagonist (arterial stenosis), SUGEN; pemirolast; pentosan polysulfate sodium; pentoxifylline; PEP-14; peripheral arterial disease therapy, Light Sciences; PF-3052334; PF-3185043; PF-3491165; PF-807925; photosensitizers (restenosis/atherosclerosis), Miravant; PI 3 kinase inhibitors, Pfizer; pioglitazone; placental expanded stem cell therapy (PLX cells, ischemia/autoimmunity), Pluristem; Plasmin (plasma-derived, peripheral arterial occlusion/ischemic stroke), Talecris Biotherapeutics; PN-271; polymer formulation (NO), University of Akron; polysulphonic acid derivatives, Fuji; PPAR alpha agonists (atherosclerosis), Merck & Co; PPAR delta agonists (dyslipidemia/diabetes/obesity/atherosclerosis), Astrazeneca; PPAR gamma agonists, GlaxoSmithKline; PPAR gamma modulators (inflammation, atherosclerosis, or diabetes), Angelini Pharmaceuticals; PPAR modulators, Ligand/Lilly; PPARalpha agonists (atherosclerosis/dyslipidemia), Bristol-Myers Squibb; PR-109; PR-86; pravastatin; PRB-01022; Preverex; pro-Apo AI; probucol (restenosis), Daiichi; PROLI/NO; propentofylline; protease activated receptor-1 antagonist (atherothrombosis), LG Life Sciences; PRT-201; PSI-421; PSI-697; PTI-101; PTR-709-A; PVS-10200; QRS-10-001; quinuclidine squalene synthase inhibitors, Zeneca; R-211945; R-755; radiolabeled VEGF (cancer), Sibtech/Stanford; raloxifene analogs, Lilly; rawsonol; raxofelast; recombinant human histone H1.3+siRNA apoB100 (atherosclerosis), SynBio; recombinant human interferon gamma receptor antagonists (graft arteriosclerosis), Tigo; relaxin (controlled release), Connetics; Restenex; restenosis radiotherapy, Angiogene; restenosis therapeutics, Cerylid; restenosis therapy, Ciba/Chiron/Focal; revacept; reveromycin-A, RIKEN; reverse cholesterol transport compounds, Fournier; reversible Lp-PLA2 inhibitors, GlaxoSmithKline; reviparin sodium; r-Factor VIIa (modified), Novo Nordisk; ribozymes (restenosis), Ribozyme; rifalazil; rilapladib; rilonacept; rimonabant; Ro-16-6532; Ro-43-8857; ROCK-1 inhibitors (atherosclerosis), MSD; ROR alpha modulators (diabetes/atherosclerosis), Orphagen; rosiglitazone; rosuvastatin; rovelizumab; roxifiban; RP-23618; RP-64477; RP-70676; RP-73163; RPR-101511a; RPR-101821; RPR-127963E; RS-93427; RSC-451061; RUS-3108; RVX-208 (oral, plaque regression), Resverlogix; RWJ-58259; S(T15) SuperAntibody (atherosclerosis), InNexus; S-12340; S-2467; S-2468; S-2E; S-31354; S-7 cell adhesion peptide (restenosis/diabetes mellitus/multiple sclerosis), Transition Therapeutics; SAH-49960; sapropterin dihydrochloride (oral, phenylketonuria), BioMarin/Merck Serono; SAR-106881; sarpogrelate hydrochloride; sarpogrelate hydrochloride (sustained release, chronic arterial occlusions), DreamPharma; SB-204990; SB-209670; SB-222657; SB-253514 analogs, GlaxoSmithKline; SB-332235; SB-435495; SC-57345; SC-69000; SC-71952; Sch-13929; Sch-46442; Sch-48461; Sch-53079; Sch-59498; SCH-602539; SDZ-267-489; SDZ-268-198; SDZ-268-445; SDZ-268-449; SDZ-268-596; SDZ-HDL-376; SDZ-MTH-958; setileuton; signal transduction inhibitor (artherosclerosis), D Western Therapeutics Institute; simvastatin+rimonabant, sanofi-aventis; siRNA anti-HMGB1 (restenosis/atherosclerosis), Bio3; sitagliptin+atorvastatin (diabetes, atherosclerosis), Merck & Co; SKF-97426; SKF-98016; SKL-14763; SKL-DES; SLV-342; SM-256; smooth muscle cell proliferation inhibitors, Wyeth; SMP-797; sodium nitrite (oral, peripheral arterial disease/diabetic foot ulcer), TheraVasc; SOL-02; somatostatin receptor 1 and 4 agonists (oral), Juvantia; sphingosine kinase inhibitors, Sankyo; sphingosine-1-phosphate modulators (cancer/vascular injury/restenosis/autoimmune/angiogenesis disorders), Kreios Pharma; SPM-5185; SQ-30404; SQ-30517; SQ-32709; SQ-33600; squalene synthase inhibitors (atherosclerosis), Bayer; squalene synthase inhibitors, Sandoz; squalene synthetase inhibitors (antihypercholesterolemia), Eisai; squalene synthetase inhibitors, Pfizer; squalestatin 1, Glaxo; squalestatin-1 analogs, Glaxo; SR-12813; SR-45023A; SR-74829i; SR-BI gene therapy, SB; strontium ranelate (oral, osteoporosis/inflammatory disease/periodontitis/atherosclerosis), Emory University; SU-11218; succinobucol; SUN-C-8257; sustained release incrementally modified drug (atheroslerosis), Daewoong; sustained-release anagrelide (oral, arteriosclerosis), Revitus; SV-618; SY-162; T-250; T-2591; t2c-001; t2c-002; T-686; TA-7552; TA-993; taberminogene vadenovec; tagatose; TAN-2177; TAS-301; TEI-6522; TEI-6620; TEI-8535; terutroban; TGF-beta elevating agent, NeoRx/University of Cambridge; TGFTX-1; Tie2-targeting siRNAs (atherosclerosis, diabetes, inflammation, cancer), Alnylam; TIMP-4 (restenosis), Transgene/HGS; tiplasinin; tiqueside; tirasemtiv; tirofiban; TKI-963; TMP-153; torcetrapib; torcetrapib+atorvastatin; TP-9201; tranilast; tranilast derivatives, Japan Energy Corp; trans sodium crocetinate, Diffusion Pharmaceuticals; trelanserin; trimerized apolipoprotein A-I, Borean; triple PPAR alpha/gamma/delta agonists (diabetes/dyslipidemia/atherosclerosis), Bayer; trombodipine; tyrosine kinase inhibitors, Pfizer; tyrosine kinase inhibitors, Sugen; U-0126; U-73482; U-76807; U-86983; U-9888; UDCA analogs, Schering-Plough; UK-122802; UK-399276; umirolimus stent (BioMatrix, restenosis), Biosensors; ureido fibrate analogs, Glaxo Wellcome; urokinase inhibitors (metastasis), 3-Dimensional Pharmaceuticals/Berlex; VAN-10-4-eluting stent, University of Strathclyde; Vascular-HSV; VB-201; VEGF/FGF antagonists, 3DP; VEGF-2 DNA vaccine (oral, atherosclerosis), LACDR; vexibinol; VINP-28; VIT-100; vitronectin antagonist, Bayer; vitronectin antagonists, BMS; vitronectin antagonists, GSK; vitronectin antagonists, Uriach; vitronectin receptor inhibitors, Wyeth; VLA-4/VCAM antagonists (inflammation), Elan/Wyeth; VLTS-589; VLTS-934; VM-202; VMDA-3601; VRI-1; VT-111; VT-214; VULM-1457; WAY-12175; WAY-121898; WAY-125147; WHI-P164; WYE-672; XJP-1; XL-652; XP-368; XT-199; YM-16638; YM-17E; YM-750; YSPSL; YT-146; Z2D3; Z-335; zaragozic acid A derivatives, Merck; zaragozic acid A, Merck & Co; zaragozic acid D, Merck; ZCL-4; ZD-9720; ZFP-VEGF; ZM-250462; ZM-97480; zotarolimus drug eluting stent (restenosis), Abbott; zotarolimus drug eluting stent (restenosis), Medtronic; ZYN-162; Zyn-linkers technology, Zynaxis; and the like.

In some embodiments of this and other aspects of the invention, the pharmaceutically active agent is an agent for treatment of atherosclerosis. Exemplary agents for treatment of atherosclerosis include, but are not limited to, 11beta-hydroxysteroid dehydrogenase-1 (HSD1) inhibitors, Merck & Co; 15-LO inhibitors, Bristol-Myers Squibb; 2,3-dioxoindoline, Qingdao University; 2164U90; 2NTX-99; 3,4-di(OH)-hydrocinnamante derivatives (oral, hyperlipidemia/atherosclerosis), KRIBB; 447C88; 568859; 99mTc-anti-ED-B; 99mTc-P215; A-87049; ABCA1/ApoA1 (atherosclerosis), Gilead Palo Alto; AC-3056; ACAT inhibitor (atherosclerosis), Kyoto; ACAT inhibitors (atherosclerosis), Takeda; ACAT inhibitors, Azwell; ACAT inhibitors, Kyowa Hakko Kogyo; ACAT inhibitors, Schering-Plough; acetylsalicylic acid+simvastatin (atherosclerosis), HanAll Biopharma; acifran; acitemate; ACP-501; acyl-CoA cholesterol acyltransferase inhibitor/diacylglycerol acyltransferase inhibitor/apolipoprotein-A1 stimulator (atherosclerosis), Kyoto; adiponectin mimetics (oral, type 2 diabetes/atherosclerosis/muscle metabolic diseases), Rigel Pharmaceuticals; ADR-7; AGI-3; AGI-H1; AGI-H-15; AHRO-001; AJ-814; AL-0671; amyloid modulators (type 2 diabetes/atherosclerosis), Crossbeta Biosciences; ANG-1170; anticholesterolemics, Pfizer; APA-01+atorvastatin (atherosclerosis), Phosphagenics; apical sodium-dependent bile acid transporter inhibitors (atherosclerosis), Sankyo; ApoA1 upregulating agents (atherosclerosis), GSK; apolipoprotein AI analogs, Fournier; Apovasc; APP-018; ARI-1778; arNOX inhibitors (oral, atherogenesis), NOX Technologies; aspalatone; Astenose; ATH-03; Atherocort; atherogenesis preventative therapy (atherosclerosis), RxBio; atherosclerosis therapy, Allelix/Fournier; atherosclerosis therapy, Aventis Gencell/INSERM; atherosclerosis therapy, Cue Biotech; atherosclerosis therapy, Millennium/Lilly; atherosclerosis therapy, Rhone-Poulenc Rorer; atherosclerosis/rheumatoid arthritis agents (sustained release/CTP), PROLOR Biotech; ATI-5261; atorvastatin+acetylsalicylic acid (atherosclerosis), HanAll Biopharma; atreleuton; ATZ-1993; avasimibe; AVE-9488; AVEX-1; AVT-06; axitirome; AY-9944; azalanstat; AZM-008; barixibat; BAY-1006451; BAY-38-1315; BAY-60-5521; BB-476; bervastatin; BI-204; BIBB-515; BIBX-79; Biglycan; bile acid inhibitors, Hoechst; Bio-Flow; Bioral ApoA1; BMS-180431; BMS-183743; BMS-188494; BMS-192951; BMS-197636; BMS-200150; BMS-212122; BMS-582949; BMS-753951; BMS-779788; BP-42, Toyama; c-1602; c-2447; c-8834; canakinumab; Capiscint; cardiovascular disease therapeutic, Lexicon/Abgenix; carvastatin; CCR2 antagonist (atherosclerosis), GlaxoSmithKline; CCR2 antagonists, Incyte/Pfizer; CCX-915; CD36 receptor-specific hexarelin analogs, Ardana; cerivastatin; CETi-1; CETP inhibitor, Sandoz; CETP inhibitors (atherosclerosis), Merck & Co; CETP inhibitors (dyslipidemia), Bayer/Merck; CETP inhibitors, Pfizer; CETP inhibitors, Schering-Plough; CGP-43371; CGS-23425; CGS-24565; chemotaxin inhibitor, CV Therapeutics; chitosan ester (atherosclerosis), Ocean University of China; Cholazol; cholesterol absorption inhibitors, Schering-Plough; cholesteryl ester transfer protein inhibitors (hyperlipidemia/atherosclerosis), Lilly; chymase inhibitors, Dainippon Sumitomo; CI-101; CI-976; CI-999; ciprofibrate; CL-277082; CL-283546; CL-283796; clopidogrel+acetylsalicylic acid (oral, atherosclerosis), Dong-A; COR-2; COR-3; CP-105191; CP-113818; CP-230821; CP-340868; CP-532623; CP-800569; CP-83101; CP-88488; CPG-603; CRD-510; crilvastatin; CS-8080; CSL-111; CTCM-163; CVT-634; CVX-210-H; CXCR2 antagonists, Fournier Pharma; CYC-10424; cyclodextrin derivatives, AMRAD; D-11-1580; dalvastatin; darapladib; DE-112; decarestrictine D; dehydroepiandrosterone, Jenapharm; DGAT inhibitors (atherosclerosis), AstraZeneca; DMP-565; Docosixine; DRF-4832; DRL-16805; DRL-17822; DuP-128 analogs, DuPont; E-5050; E-5324; efipladib; eflucimibe; eldacimibe; endothelial lipase antisense inhibitors (atherosclerosis), Isis; EP-1242; ESP-24218; estrogen receptor beta modulators (triazine), GlaxoSmithKline; ET-642; ETC-1001; ETC-588; ETX-6107; F-10863A; F-1394; F-2833; farnesoid X receptor agonists, Allergan; farnesoid X receptor antagonists, Allergan; FCP-3P1; FE-301; first-generation niacin receptor agonists (oral, atherosclerosis), Merck/Arena; fluvastatin; fosinopril; fostamatinib; FR-129169; FR-145237; FR-186054; FR-186485; FY-087; gadolinium texaphyrins (imaging, atherosclerosis), Pharmacyclics; GAL T-2 inhibitors (restenosis/PKD/atherosclerosis/inflammation/AMD), Amalyte Pharmaceuticals; gantofiban; gemcabene; gemfibrozil analogs, Novartis; glenvastatin; glutathione peroxidase mimetics (oral, atherosclerosis), Provid; glyco-S-nitrosothiols, University of Miami; goxalapladib; GPR25 antagonists (myocardial infarction/stroke/atherosclerosis), Omeros; GR-328713; GW-2331; GX-401 program; H-290/30; HDL cholesterol enhancers (atherosclerosis/coronary artery disease), Wyeth; HDL delipidation therapy (LSI-5955, atherosclerosis), Lipid Sciences; HDL elevating/lipid regulating agents, Pfizer/Esperion; hE-18A; HL-004; HMG-CoA inhibitors, BMS; HMG-CoA inhibitors, Pfizer; HMG-CoA reductase inhibitors, Glaxo; hypolipemic agents, Aventis/Amylin; ICI-245991; icrucumab; ILlaQb therapeutic vaccines (atherosclerosis), Cytos; immunotherapeutic vaccine (atherosclerotic plaque), Aterovax/INSERM; INCB-3284; integrin alpha-V/beta-3 receptor mAb (atherosclerosis), Vascular Pharmaceuticals; interferon beta gene therapy (electroporation/TriGrid/im, multiple sclerosis), Ichor Medical Systems; INV-400 series; iroxanadine; isradipine; J-104123; jumonji-domain-containing-3 modulators (cancer/allergy/atherosclerosis), Osaka University; K-604; KC-706; KD-025; KF-17828; KH-01500; KH-01501 series; KI-0002; kininogen domain 5 peptides, DuPont; KM-011; KY-331; KY-455; L-166143; L-659699; L-669262; L-731120; lacidipine; laropiprant+extended-release niacin (coronary artery disease/atherosclerosis), Merck & Co; laropiprant+extended-release niacin+simvastatin (coronary artery disease), Merck & Co; LCAT gene therapy, NIH; Lck tyrosine kinase inhibitors, BMS; LDL gene, Genetic Therapy; lecimibide; lercanidipine; LF-08-0133; LF-13-0491c; lifibrol; lipid modulators, BioCache; lipid peroxidation inhibitors, Servier; lipoprotein a inhibitors, Pfizer; LK-903; losmapimod; lovastatin; LS-3115; luteusin-C; LXR agonists (Alzheimers disease), Anagen Therapeutics; LXR agonists (atherosclerosis/dyslipidemia/Alzheimer's disease), AstraZeneca; LXR agonists (dyslipidemia/atherosclerosis/diabetes), Tanabe; LXR modulators (atherosclerosis), Vitae Pharmaceuticals; LXR modulators (hypercholesterolemia/atherosclerosis), Phenex; LXR modulators (inflammation), Karo Bio/Pfizer; LY-2157299; LY-295427 analogs, Lilly; LY-674; lysosomal acid lipase, LSBC; MAP kinase inhibitors (inflammation/pain/fibrosis), Allinky; MBX-2599; MC-031; MC-032; MC-033; MC-034; MCP-1 inhibitors, Millennium/Pfizer; MCP-1 inhibitors, Roche/Iconix; MDCO-216; MDL-28815; MDL-29311; MIF antagonists (inflammation), Cortical; mimic HMGB-1 antibodies (restenosis/atherosclerosis), Bio3; misoprostol; MK-0736; MK-1903; MK-6213; MKC-121; MLN-1202; MMP-12 inhibitors (atherosclerosis), CEA; MMP-13 inhibitors (arthritis), Wyeth; molecularly imprinted polymers (hyperphosphatemia), Semorex; monoclonal antibody (atherosclerosis), Scotgen; motexafin lutetium; MT1-MMP inhibitors, 3DP; MTP inhibitors, Leiden University; myeloperoxidase inhibitors (oral/small molecule, atherosclerosis), Torrey Pines; N,N′-diacetyl-L-cystine; N-1177-iv; N-4472; naAGs (inflammation/cancer/atherosclerosis/AMD/COPD), SelectX; nanotherapeutics (breast cancer, lung cancer, infectious diseases, sepsis, atherosclerosis), SignaBlok; NB-598; NI-0401; nicotinic acid 1 receptor (GPR109A) agonists, Merck; NIK modulators, Celgene; Nimoxine; NMDA receptor antagonists (atherosclerosis), University of Nebraska Medical Center; NO synthase modulators, CNRS; NPH-4; NTE-122; OPC-35564; Org-13061; P-06103; P-06133; P-06139; P-0654; P-2202; P2Y12 inhibitor (oral, atherothrombosis), LG Life Sciences; P-773; P-947; PAI-1 antagonists, 3DP; pamaqueside; parogrelil; PD-089828; PD-098063; PD-129337; PD-13201-2; PD-132301-2; PD-135022; PD-146176; PD-148817; PD-161721; PD-166285; PDE4/MMP inhibitors, Rhone-Poulenc; PDGF receptor kinase inhibitors, AEterna Zentaris; PDGF receptor program, Millennium; pentosan polysulfate sodium; PEP-14; PF-3052334; PF-3185043; PF-3491165; PF-807925; photosensitizers (restenosis/atherosclerosis), Miravant; PI 3 kinase inhibitors, Pfizer; pioglitazone; polysulphonic acid derivatives, Fuji; PPAR alpha agonists (atherosclerosis), Merck & Co; PPAR delta agonists (dyslipidemia/diabetes/obesity/atherosclerosis), Astrazeneca; PPAR gamma agonists, GlaxoSmithKline; PPAR gamma modulators (inflammation/atherosclerosis/diabetes), Angelini Pharmaceuticals; PPAR modulators, Ligand/Lilly; PPARalpha agonists (atherosclerosis/dyslipidemia), Bristol-Myers Squibb; PR-109; PR-86; pravastatin; PRB-01022; Preverex; pro-Apo AI; protease activated receptor-1 antagonist (atherothrombosis), LG Life Sciences; PSI-421; PSI-697; PTR-709-A; QRS-10-001; quinuclidine squalene synthase inhibitors, Zeneca; R-211945; R-755; radiolabeled VEGF (cancer), Sibtech/Stanford; raloxifene analogs, Lilly; rawsonol; raxofelast; recombinant human histone H1.3+siRNA apoB100 (atherosclerosis), SynBio; revacept; reveromycin-A, RIKEN; reverse cholesterol transport compounds, Fournier; reversible Lp-PLA2 inhibitors, GlaxoSmithKline; rifalazil; rilapladib; rilonacept; rimonabant; Ro-16-6532; ROCK-1 inhibitors (atherosclerosis), MSD; ROR alpha modulators (diabetes/atherosclerosis), Orphagen; rosiglitazone; rosuvastatin; RP-23618; RP-64477; RP-70676; RP-73163; RPR-101821; RS-93427; RSC-451061; RUS-3108; RVX-208 (oral, plaque regression), Resverlogix; S(T15) SuperAntibody (atherosclerosis), InNexus; 5-12340; S-2467; S-2468; S-31354; SAH-49960; SB-204990; SB-222657; SB-253514 analogs, GlaxoSmithKline; SB-332235; SB-435495; SC-57345; SC-69000; SC-71952; Sch-13929; Sch-46442; Sch-48461; Sch-53079; SCH-602539; SDZ-267-489; SDZ-268-198; SDZ-268-445; SDZ-268-449; SDZ-268-596; SDZ-HDL-376; setileuton; signal transduction inhibitor (artherosclerosis), D Western Therapeutics Institute; simvastatin+rimonabant, sanofi-aventis; siRNA anti-HMGB1 (restenosis/atherosclerosis), Bio3; sitagliptin+atorvastatin (diabetes, atherosclerosis), Merck & Co; SKF-97426; SKF-98016; SKL-14763; SLV-342; SOL-02; SPM-5185; SQ-30404; SQ-30517; SQ-32709; SQ-33600; squalene synthase inhibitors (atherosclerosis), Bayer; squalene synthase inhibitors, Sandoz; squalene synthetase inhibitors (antihypercholesterolemia), Eisai; squalene synthetase inhibitors, Pfizer; squalestatin 1, Glaxo; squalestatin-1 analogs, Glaxo; SR-12813; SR-45023A; SR-74829i; SR-BI gene therapy, SB; strontium ranelate (oral, osteoporosis/inflammatory disease/periodontitis/atherosclerosis), Emory University; succinobucol; SUN-C-8257; sustained release incrementally modified drug (atheroslerosis), Daewoong; T-2591; T-686; TA-7552; tagatose; TAN-2177; TEI-6522; TEI-6620; TEI-8535; terutroban; TGF-beta elevating agent, NeoRx/University of Cambridge; TGFTX-1; Tie2-targeting siRNAs (atherosclerosis, diabetes, inflammation, cancer), Alnylam; tiplasinin; tiqueside; TMP-153; torcetrapib; torcetrapib+atorvastatin; trimerized apolipoprotein A-I, Borean; triple PPAR alpha/gamma/delta agonists (diabetes/dyslipidemia/atherosclerosis), Bayer; trombodipine; U-0126; U-73482; U-76807; U-9888; UDCA analogs, Schering-Plough; UK-122802; UK-399276; ureido fibrate analogs, Glaxo Wellcome; VB-201; VEGF-2 DNA vaccine (oral, atherosclerosis), LACDR; vexibinol; VINP-28; VLA-4/VCAM antagonists (inflammation), Elan/Wyeth; VULM-1457; WAY-12175; WAY-121898; WAY-125147; WHI-P164; WYE-672; XJP-1; XL-652; XP-368; XT-199; YM-16638; YM-17E; YM-750; Z2D3; zaragozic acid A derivatives, Merck; zaragozic acid A, Merck & Co; zaragozic acid D, Merck; ZCL-4; ZD-9720; ZM-250462; ZM-97480; and any combinations thereof.

In some embodiments of this and other aspects of the invention, the therapeutic agent is an agent for treatment of sepsis. Exemplary agents for treatment of sepsis include, but are not limited to, 2-aminotetraline derivatives (brain inflammation), Sigma-Tau; 3936W92; 3G-12-scFv; 6343; A-84643; AB-022; AB-103; ABC-88; ABT-299; afelimomab; AFX-300 series, Aphoenix; alpha 2A adrenoceptor antagonist (sepsis), TheraSource; alpha-v/beta-5 monoclonal antibody, Stromedix; ALT-836; anakinra; anti-CD11a MAb, Geneva University; anti-inflammatory protein (severe sepsis/myocardial infarction), Celdara; anti-iNOS mAbs (sepsis), DSX Therapeutics; anti-sepsis peptides, Agennix; antisepsis therapy, Huons; antithrombin alfa; antithrombin III, Aventis Behring; apadenoson; APG-101; apolipoprotein AI analogs, Fournier; AR-9281; ATL-193; AVI-4014; AZD-9773; B-0202; B-214; bimosiamose (intravenous formulation/injectable formulation, acute lung injury), Revotar; bovine alkaline phosphatase (iv, renal failure), AM-Pharma; bradykinin antagonists, Scios; C-10, Interthyr; camel-derived anti-macrophage immune-activating enzyme antibodies (oral, septic shock), Canopus; CAP-18; caspase inhibitors (cancer), EpiCept; CDP-571; cefepime; cefotiam; ceftriaxone; cefuroxime axetil; CKD-712; CL-184005; clinafloxacin; clindamycin; CN-16; complement component 3a antagonists, RWJ; CP-0127; CS-4771; CSL-111; CT-500; CV-3988; CY-1787; CY-1788; CyP (inflammatory disease/reperfusion injury/sepsis), Bluegreen; CYT-107; D-609; dalbavancin; daptomycin; diaspirin cross-linked hemoglobin, Baxter; dipeptidyl peptidase I inhibitors (sepsis), Arpida; doramapimod; doripenem; drotrecogin alfa; DW-286; DY-9973; E coli verotoxin disease therapy, Select Therapeutics; E-5531; E-7016; EA-230; edobacomab; EI-1507-1; ERB-196; ERB-257; eritoran; ertapenem; FE-202158; flomoxef, Shionogi; fluorofenidone; free radical scavengers (sepsis/community-acquired pnemonia), Lantibio; FX-107; gamma interferon antagonist, Genzyme Molecular Oncology; GCH-01; GI-5402; ginkgolide B; GK-04489; Glyco-23; GM-1595; GP-1-515; GR-194444; GR-270773; GR-270773, Glaxo Wellcome; group B streptococcal vaccine, LigoCyte; GYKI-66430; HBN-3; heparin binding protein, Novo Nordisk; HMGB-1 antagonists, Cornerstone Therapeutics; human AM/AMBP-1 (ischemia reperfusion injury), TheraSource; humanized antitissue factor monoclonal antibodies, Centocor; IC-14; ICE inhibitors, Pfizer/Abbott; IFX-1; IL-13, Sanofi; ilodecakin; imipenem+cilastatin; IND-005; IND-006; INNO-202; INO-1001; inter alpha inhibitor proteins (sepsis), BioThera Biologics; ISO-1; ISU-201; JSdLPS/OMP, University of Maryland; J5-OMP; JTE-607; KPE-05001; KRX-211; L-161240; L-97-1 (intravenous, sepsis/pneumonic plague), Endacea; LAS-30989; lenercept; levocarnitine; lexipafant (iv formulation), DevCo Pharmaceuticals; lificiguat; linezolid; lipid A vaccine (sepsis), Scripps; lisofylline; L-NMMA, Fujisawa; LPS inhibitors (recombinant peptide/fragment, sepsis), NUS; LPS neutralizing recombinant tetrameric S3 peptide (sepsis), NUS; LY-215840; M-62812; MDI-P; MDL-101002; MFH-147; MG-96077; MIF inhibitors, Picower; minopafant; monocyte colony inhibitory factor, HGS; MPL-S; MRL-953; MSI-136; MSM-236; N-2733; nanotherapeutics (breast cancer, lung cancer, infectious diseases, sepsis, atherosclerosis), SignaBlok; NAV-838; NCY-118; nebacumab; nerelimomab; NK-modulating CD27 antibodies (viral infection/bacterial infection/sepsis), HZI; NO synthase inhibitors, Merck & Co; NO synthase modulators, CNRS; NOX-100; NPC-15199; NPC-15669; Ochrobactrum intermedium LPS (sepsis), Diomune; OLX-514; ONO-1714; opebacan; oritavancin; P-13; P-7, 13Therapeutics; paraoxonase, Pfizer; PARP inhibitors, Crimson Pharmaceutical; pazufloxacin; peptide therapy (septic shock), MorphoSys; phospholipase A2 inhibitors (endotoxic shock/inflammation) Glaxo Wellcome; piperacillin+tazobactam (injectable), Wyeth/Toyama/Taiho; PMX-622; PN-1561; pralnacasan; Procysteine; protein kinase C inhibitors (1), Lilly; PTS-508a; QRS-5-005; RAS-111; recombinant Slit-2-D1-D2-Fc (ALI/ARDS/sepsis), Navigen Pharmaceuticals; recombinant Slit2N (viral hemorrhagic fever/ARDS/anthrax infection/sepsis), Navigen Pharmaceuticals; resatorvid; RGN-137; r-hdl; rocepafant; rPAF-AH; RR-1; sargramostim; SB-203347; SB-249417; Sepcidin; Sepsicillin; sepsis program, Dong Wha; sepsis therapy (bacterial gene silencing), CytoGenix; sepsis therapy, Hansa Medical; septic shock therapy, Biorex; serine protease inhibitors, SuperGen/Wichita; SJC-13; SPC-702; sPLA2 inhibitors, Lilly; SRI-63-675; ST-899; STEBVax; superantigen toxin therapy (antibodies), Callisto; talactoferrin alfa; TCV-309; teicoplanin; temocillin; Tenecrin; THG-315; tifacogin; tilarginine; TLR4/MD-2 monoclonal antibody therapy (endotoxic shock), NovImmune; TNF alpha inhibitors, Synta; TNF receptor, Roche; TNFalpha-induced apoptosis inhibitors, Genzyme; TREM-1 inhibitors (non-small cell lung cancer, breast cancer, sepsis, hemorrhagic shock), SignaBlok; TREM-1 targeting compounds, Novo Nordisk; trichodimerol; TSS-HIG; UK-91473; UR-12633; vancomycin; varespladib (iv formulation, acute chest syndrome in sickle cell anemia), Anthera; VGV-S; VGX-300; VGX-350; VGX-750; vitamin D3 analogs, BioXell; VTR-4; VX-166; VX-799; WCK-771; XMP-600; Y-40138; and the like.

In some embodiments, the therapeutic agent is an anti-cancer agent or a cancer vaccine. Exemplary anti-cancer agents and vaccines include, but are not limited to, 9-peptide vaccine (breast cancer), University of Virginia; A1-mafodotin; abagovomab; ABTSC-DC vaccine, Cellonis; AC-01; ACH-1625; Ad/PSA; Ad5 [E1-, E2b-]-HER2/neu vaccine, Etubics/Duke Comprehensive Cancer Center; Ad5 vector vaccine targeting E6/7 tumor associated antigen, (E.C7 cell line, HPV associated head and neck cancer), Etubics/South Dakota University; Ad5f35-LMPd1-2-transduced autologous dendritic cells (EBV-associated cancer), NCI; ADC-1009; adenovirus vector E2b-deleted PSA targeting vaccine (E.C7 cell line, prostate cancer), Etubics; adenovirus vector E2b-deleted WT-1 gene targeting vaccine (E.C7 cell line, cancer), Etubics; adenovirus-mediated immunotherapy (melanoma), Zurich; Ad-HPV E6/E7 vaccine, VectorLogics; Ad-PSMA vaccine, VectorLogics; ADVAX; ADXS-HER2; ADXS-HPV; Adxs-LmddA159; ADXS—PSA; AE-08; AE-298p; AE-37, Antigen Express; AE-37/GP-2 vaccine (cancer), Antigen Express; AEA-35p; AEH-10p; AE-M; AE-O; AEZS-120; AFTVac; AG-858; agatolimod; AGI-101H; AGS-003; AGS-005; AGS-006; AHICE; algenpantucel-L; allogeneic cell vaccine (non-small cell lung cancer), University of Kentucky; allogeneic cellular melanoma vaccine, New York Medical College; AlloStim (infusion formulation, hematological neoplasms), Immunovative; AlloStim+autologous chaperone protein vaccine (hematological cancer), Immunovative; alpha-fetoprotein cancer vaccine (hepatocellular carcinoma), Kite; alpha-lactalbumin vaccine (breast cancer), Cleveland Clinic; alpha-type-1 polarized dendritic cells (chronic lymphocytic leukemia), University of Pittsburgh; ALT-212; ALVAC-CEA/B7.1; ALVAC-GM-CSF; ALVAC-gp100 melanoma vaccine, Aventis Pasteur; ALVAC-KSA; ALVAC-MAGE-1/MAGE-3 skin cancer vaccine, sanofi-aventis; AML vaccine (JuvaVax), Juvaris; amolimogene bepiplasmid; AMP-224; anti-angiogenesis vaccine (anti-VEGF-a), Immunovo; anticancer vaccines, Bioleaders; anti-CD3 activated vaccine-primed lymphocytes (cancer), University of Michigan; Anti-CEA antibody, Albert Einstein; antigen-pulsed dendritic cell vaccine (melanoma), Hadassah Medical Organization; antigen-pulsed dendritic cell vaccine (pancreatic cancer), Musashino University; antigen-specific melanoma vaccine, Genzyme Molecular; anti-idiotype HER2 vaccine (cancer), Institut de Recherche en Cancerologie de Montpellier; anti-mammaglobin vaccine (breast cancer), Washington University in St Louis; antimetastasis therapeutic vaccine, Protherics; anti-PTT273 vaccine (prostate cancer, ADX-40 adjuvant), Adjuvantix/Pro-Cure; anti-TACA cancer vaccine (GlycoMim), TFChem; anti-TEM-1 DNA vaccine (cancer), University of Pennsylvania; anti-WT-1 cancer vaccine (Listeria vector), Advaxis; ANZ-100; ANZ-521; AP-1903; AP-1903-activated MyD88/iCD40 dendritic cell vaccine (cancer), Bellicum; APC-8020; APC-80TR; ApoVax104-HPV; ARGENT (prostate cancer therapy), ARIAD; ASIbc1 vaccine, Intracel; ASP-0113; astuprotimut-r, GlaxoSmithKline; atazanavir; autologous anti-gp100 T-cell receptor gene-engineered peripheral blood lymphocytes (melanoma), National Cancer Institute; autologous dendritic cell therapy (cancer), ML Laboratories; autologous dendritic cell vaccine (HIV-1 infection), Institut de Recherche sur les Vaccins et l'Immunotherapie des Cancer et du Sida; autologous dendritic cell vaccine (leukemia), Karolinska Institutet; autologous dendritic cell vaccine (renal cancer), Trimed Biotech; autologous dendritic cell vaccines (cancer), tella; autologous dendritic cell-tumor cell fusion vaccine (gastrointestinal cancer), Teikyo University; autologous Hsp70 cancer vaccine, Kyoto University; autologous melanoma cell vaccine (neoplasm), Dana-Farber; autologous multiple antigen dendritic cell vaccine (PSA, PSMA, prostein, survivin, Trp-p8), Technische Universitat Dresden; autologous NY-ESO-1-targeting dendritic cell vaccine (cancer), Roswell Park; autologous renal cell carcinoma vaccine, Dartmouth-Hitchcock Medical Center; autologous therapeutic cancer vaccine, TVAX Biomedical; autologous tumor cell vaccine (leukemia), NCI; autologous tumor cell-TLR9 agonist vaccine (colorectal cancer), University of Stanford; AVX-701; azacitidine; B7-1 gene therapy (in vivo/Ig G), Georgetown/Imperial College; B7-1 gene therapy, University of Wisconsin; bacteriophage vaccine (lymphoma), Apalexo; bacteriophage vaccine (multiple myeloma), Apalexo; balapiravir; B-cell lymphoma DNA vaccine, Cancer Research Ventures; BCG vaccine, Organon; BCL-002; BCL-003; BCL-004; BCL-005; Bcr-Abl DNA vaccine expressing GM-CSF and IL-12 (leukemia), Mologen; belagenpumatucel-L; bendamustine; BhCG vaccine (cancer), UCL/Vaxcel; BHT-3009; bioerodible DDS (vaccines); BiovaxlD; BIWB-1; BIWB-2; BN-500001; BN-600013; BP-16; BPX-101; brain tumor vaccine, IRC; breast cancer topical vaccine, Vaxin; breast cancer vaccine, MD Anderson Cancer Center; BrevaRex; B-Vax; CA-9-targeted autologous cell therapy (cancer), Dendreon; CA9-targeted fusion protein (Listeria vaccine, cancer), Advaxis; CAD-106; cadi-05; cancer peptide antigen vaccine, Canopus BioPharma; cancer vaccine (CD1), Antigenics; cancer vaccine (measles virus), Mayo/Onyvax; cancer vaccine (prostate/breast/colon cancer), CEL-SCI (MaxPharma); cancer vaccine (VacciMax), ImmunoVaccine; cancer vaccine (VIASKIN), DBV Technologies; cancer vaccine, ApoImmune; cancer vaccine, Attogen; cancer vaccine, AVAX/Thomas Jefferson University; cancer vaccine, Galenica Pharmaceuticals/University of Alabama; cancer vaccine, Geniva; cancer vaccine, Immpheron; cancer vaccine, MCP Hahnemann Uni; cancer vaccine, Ohio State University; cancer vaccine, PolyMASC/Hydro Med; cancer vaccine, University of Illinois/Research Corp Technology; Canvaxin; CAP1-6D; CBD1Qb; CBI-006; CBI-008; CC-394; CD40 ligand, Celldex; CD55-targeting vaccine (cancer), Viragen; CDCA1-derived epitope peptide vaccine (HLA-A2402 restricted, prostate cancer), Iwate Medical University/Tokyo University/Oita University; cDNA vaccine (prostate cancer), Colby; CDX-1127; CDX-1307; CDX-1401; CDX-2410; CDX-301; CEA peptide-loaded dendritic cell vaccine (colorectal cancer), Osaka University; CEA RNA transfected autologous dendritic cell vaccine (cancer), Duke University; CEA(6D)-TRICOM vaccine (colorectal/lung cancer), Therion/NCI; CEA-based DNA vaccine, Vanderbilt/Scripps; CEA-dendritic cell-based vaccine, Takara; CEA-targeted autologous cell vaccine (cancer), Dendreon; CeaVac; CEL-1000; cellular vaccine (ovarian cancer), Cleveland Clinic Foundation; Cervarix; CerVax-16; cervical cancer vaccine (oral, HPV infection), Apimeds; CG-201; cHER2+VEGFR2 targeting vaccine (Listeria vector, cancer), Advaxis; chimeric TRP protein vaccine (melanoma), ImClone; Chlamydia molecular vaccine (infertility/infection), BC Cancer Agency; choriogonadotropin alfa; ChronVac-C; CIGB-228; CIGB-247; CL-2000; CML vaccine, Breakthrough Therapeutics; CMV RNA transfected autologous dendritic cell vaccine (glioblastoma), Duke University; CMVAC; CMX-001 (glioblastoma multiforme), California Pacific Medical Center; colon cancer vaccine, Immune Response Corp; COLO-Vax; COMBIG-vaccine (cancer), Immunicum; combined PR1/WT1 vaccine (leukemia), NIH; combined vaccine adjuvants (cancer), ImmuRx; contusugene ladenovec; CPG23PANC; CreaVax-HCC; CreaVax-PC; CreaVax-RCC; CRL-1005; CRM-197; CRS-207; CryoStim; CT-011; CT-201; CT-5; CTL (melanoma), Fred Hutchinson/Washington/Targeted Genetics; CTL-8004; CTP-37; CV-01; CV-07; CV-09; CV-301; CV-9103; CV-9201; CVac; CYT-003-QbG10; CYT-004-Me1QbG10; CYT-005-allQbG10; CYT-006-AngQb; CYT-007-TNFQb; CYT-009-GhrQb; CYT-014-GIPQb; cytotoxic T-lymphocyte vaccine (nanoparticle nasal, cancer), Peptagen; D-3263; daclatasvir; DC/I540/KLH vaccine (cancer), Dana-Farber; DC-Ad-CCL-21 intratumoral therapy, UCLA/Department of Veterans Affairs; DC-Cholesterol (adjuvant), Targeted Genetics/Pasteur Merieux Connaught; DC-NILV-based cancer vaccine, Immune Design; DCP-001; DCP-002; DCVax; DCVax-Brain; DCVax-Head/Neck; DCVax-Liver; DCVax-Lung; DCVax-Ovarian; DCVax-Pancreas; DCVax-Prostate; DEFB 1 stimulators (peptide, prostate cancer), Phigenix; dendritic cell immunotherapy (ovarian cancer), Life Research Technologies; dendritic cell myeloma fusions (multiple myeloma), Dana Farber/Beth Israel Deaconess; dendritic cell therapy (cancer), Binex/KunWha; dendritic cell vaccine (C5 alpha agonist adjuvant, cancer), University of Nebraska Medical Center; dendritic cell vaccine (colon tumor), ODC; dendritic cell vaccine (glioblastoma multiforme), Malaghan Institute of Medical Research; dendritic cell vaccine (head and neck cancer), University of Maryland/Hasumi; dendritic cell vaccine (injectable, head and neck cancer), National Cancer Institute; dendritic cell vaccine (melanoma), European Institute of Oncology/TTFactor; dendritic cell vaccine (melanoma), ODC; dendritic cell vaccine (melanoma), Vrije Universiteit Brussel; dendritic cell vaccine (prostate cancer), Medistem/Genelux/University of California/San Diego State University; dendritic cell vaccine (prostate tumor), ODC; dendritic cell vaccine (solid tumor), ODC Therapy/SBI Biotech; dendritic cell vaccine, GeneMedicine; dendritic cell vaccine, University of Bonn; dendritic cell/WT1 class I/II peptide vaccine (cancer), tella/Jikei University; dendritic cell-derived exosomes, Anosys; DISC/GM-CSF; DISC-PRO; DNA fusion vaccine (CEA-expressing tumors), Cancer Research UK; DNA vaccine (colorectal cancer), enGene; DNA vaccine (Derma Vax, colorectal cancer), Cellectis/Karolinska Institute; DNA vaccine (Derma Vax, lung cancer), Cyto Pulse; DNA vaccine (Derma Vax, prostate cancer), Cellectis/Karolinska Institute; DNA vaccine (intramuscular electroporation, leukemia), University of Southampton/Inovio; DNA vaccine (intratumoral/EPT, prostate cancer), University of Southampton/Inovio; DNA vaccine (melanoma), Memorial Sloan-Kettering Cancer Center; DNA vaccine (melanoma), NCI; DNA vaccines (cancer), Bio-Ker; DNA vaccines (cancer), ImmunoFrontier; DNA vaccines (cancer), Southampton University; DNA vaccines (cancer), Vaccibody; DNGR-1 antibody vaccine (cancer), CRT; DPX-0907; DPX-Survivac; Drug Name; dSLIM (colon cancer), Mologen; DV-601; E7 toxoid; E7/HSP70 DNA vaccine, Johns Hopkins University; E-7300; E747-57 peptide plus synthetic dsRNA vaccine (RNA technology, solid tumors), Mannkind Corp; EBV CTLs (EBV-associated lymphoma, nasopharyngeal carcinoma), Baylor College/Cell Medica; EBV-related Hodgkin's disease vaccine, Vaccine Solutions; EC-708; edible transgenic plant-expressed recombinant human papilloma virus vaccine (oral, HPV infection), Apimeds; EGF vaccine (cancer), CIMAB/Micromet/Biocon/Bioven; EGFR vaccine (cancer), L2 Diagnostics; EGFR-expressing Saccharomyces cerevisiae-based cancer vaccine (Tarmogen), Globelmmune; EG-HPV; EG-Vac; elpamotide; EMD-249590; emepepimut-S; Engerix B; enkastim-ev; enkastim-iv; ENMD-0996; entinostat; enzalutamide; EPICOAT cancer vaccine, Axis; Epstein Barr virus vaccine (PREPS/L-particles), Australian Centre for Vaccine Development (ACVD)/Henderson Morley; Epstein Barr-based gene therapy (intradermal, cancer), University of Birmingham; Epstein-Barr virus vaccine, Cents; EradicAide; estradiol (transdermal, micro-encapsulated), Medicis/Novavax; ETBX-011; Eukaryotic Layered Vector System; ex vivo adenosine deaminase-transduced hematopoietic stem cell therapy (ADA-SCID), GSK; F10 (neutralizing antibody, group 1 influenza A infection), Harvard Medical School/Dana-Farber Cancer Institute/XOMA/SRI International; F-50040; FANG vaccine; FAV-201; FBP-E39 vaccine (cancer), Galena Biopharma; FG-004, 4G Vaccines; fibroblast cell therapy (Parkinsons), Cell Genesys (Somatix); fibronectin extra domain A (vaccine adjuvant), Digna Biotech; fibrovax, Cytokine; folate receptor alpha-targeted therapeutic vaccine (cancer), VaxOnco/Mayo Clinic; Folatelmmune; fosamprenavir; FP-03; FPI-01; frame-shift peptide vaccine (colorectal cancer), Oryx GmbH; Freevax; fresolimumab; fucosyl-GM1-KLH; fusogenic lipids, Liposome Company; Fve polypeptide vaccine (allergy/viral infection/cancer), NUS; ganglioside vaccine (polyvalent, sarcoma), Memorial Sloan-Kettering/MabVax; Gardasil; gastrin 17C diphtheria toxoid conjugate (pancreatic cancer), Aster; gastrin synthetic peptide antigen vaccine (pancreatic cancer, TDK), Immunovo; gataparsen; GD2 ganglioside peptide mimics, Roswell Park Cancer Institute; Gemvac; gene therapy (Alzheimers), Somatix; gene therapy (anticancer), MediGene/Aventis; gene therapy (cancer), GenEra; gene therapy (cardiovascular), Somatix/Rockefeller; gene therapy (HPV), Chiron Viagene; gene therapy (HSV), Chiron Viagene; gene therapy (IL-2, cLipid), Valentis/Roche; gene therapy (prostate cancer), GenStar/Baxter; gene therapy (RTVP-1), Baylor College of Medicine; gene therapy (vaccine), ICRF/RPMS; GeneVax vaccine (cancer), Centocor; GeneVax vaccine (HIV), Wyeth/University of Pennsylvania; Genevax vaccine (lymphoma), Apollon; GI-10001; GI-4000; GI-5005; GI-6000; GI-6207; GI-6301; GI-7000; GL-0810; GL-0817; GL-ONC1; Gly-MUC1 conjugate prostate tumor vaccine, Memorial Sloan-Kettering; GM-CAIX; GM-CSF cancer vaccine, Thomas Jefferson/NCI; GM-CSF cell therapy (melanoma), University of Wisconsin; GM-CSF tumor vaccine, PowderJect; GM-CSF vaccine, Johns Hopkins; GM-CSF/B7-2 gene therapy and vaccine (CIT, cancer), Radient Pharmaceuticals/Jaiva Technologies; GM-CSF-G250 vaccine, UCLA; GM-CSF-transduced autologous cancer stem cell vaccine, Kyushu University/DNAVEC; GM-CT-01; GMDP, Peptech; GMK; GnRH immunotherapeutic, ML/Protherics; golimumab; golotimod; Gonadimmune; gp100/GM-CSF melanoma vaccine, University of Wisconsin; gp100:209-217(210M) peptide vaccine (melanoma), NCI; gp53, ImClone; gp75 DNA vaccine (melanoma), Memorial Sloan-Kettering Cancer Center; gp75 melanoma therapy, Memorial Sloan-Kettering; GPC-3298306; GPI-0100; GRNVAC-1; GrVax; GS-7977; GSK-2130579A; GSK-2241658A; GSK-2302024A; GSK-2302025A; GSK-2302032A; GSK-568893A; GV-1002; GV-1003; GVAX; GVX-3322; GX-160; GX-201 program; GX-301 program; GX-51; H pylori vaccine, Apovia; H pylori vaccines, sanofi-aventis; H1 therapeutic vaccine (liposomal, ImuXen, cancer), Xenetic Biosciences/Pharmsynthez; H1N1 influenza A vaccine (VLP), Novavax/NIAID; HCV vaccine (ISCOMATRIX), Novartis; HE-2000; HelicoVax; hepatitis B vaccine, Boyce Thompson Institute; HepeX-B; Heplisav; HER-1 vaccine (cancer), Bioven Holdings/CIMAB; HER1-VSSP vaccine (cancer), The Center of Molecular Immunology; HER-2 DNA AutoVac; HER-2 peptide-expressing DNA vaccine, Karolinska Institute; HER-2 protein AutoVac; Her-2 vaccine (anticancer), University of Alabama/Galenica Pharmaceuticals; HER-2 vaccine (cancer), L2 Diagnostics; HER-2/CEA DNA vaccine (cancer), Merck/IRBM/Inovio/Vical; HER-2/HER-1 vaccine (solid tumors), Ohio State University; HER2/neu peptide vaccine (intradermal, breast cancer), Fred Hutchinson Cancer Research Center; HER2/neu peptide vaccine (intramuscular, breast cancer), Norwell; HER-2/Neu Pulsed DC1 Vaccine (breast cancer), University of Pennsylvania/National Cancer Institute; Her-2/neu vaccine (breast cancer), Alphavax/Duke University; HER2-CAR T-cells; HER2p63-71 peptide vaccine, Mie University; HerVac; HGP-30; HGTV-43; Hi-8 PrimeBoost therapeutic HBV vaccine, Oxford Biomedica; Hi-8 PrimeBoost therapeutic melanoma vaccine, Oxford BioMedica; HIV vaccine (SAVINE), BioVax; HIV-1 gag DNA vaccine, Merck & Co; HLA-A*2402-restricted KIF20A and VEGFR-1 epitope peptide vaccine (pancreatic cancer, subcutaneous), Juntendo University School of Medicine; HLA-A, B7.1-transfected adenocarcinoma vaccine, University of Miami; Homspera; Homspera (oral, influenza), ImmuneRegen; hormone-independent vaccine (prostate tumor), Zonagen; HPV 16/18 vaccine (bivalent), Xiamen Innovax Biotech; HPV E7 cancer vaccine (liposomal, VacciMax), ImmunoVaccine Technologies; HPV E7/calreticulin DNA vaccine (gene gun), Johns Hopkins University; HPV vaccine (AAV vector, AAVLP program), MediGene; HPV vaccine (cancer), Fraunhofer; HPV vaccine (cancer/HPV infection/prevention), Coridon; HPV vaccine (iBioLaunch), iBio; HPV vaccine (monovalent), Merck & Co; HPV/cervical cancer vaccine program, Bionor Immuno; HPV-16 E7 lipopeptide vaccine, Tufts University School of Medicine; HPV-16 E7 vaccine (cancer), NCI; HPV-16-E7, Loyola University; HS-110; HS-210; HS-310; HS-410; HSP105 antigen peptide dendritic cell vaccine (cancer), Medinet; hsp110 vaccine, Roswell Park; HspE7; Hsp-HIV antigen fusion therapy, StressGen; HSPPC-56; HSPPC-90; HSV vaccine (LEAPS), CEL-SCI (MaxPharma)/Ohio University; human and mouse gp100 DNA plasmid vaccines (melanoma), Memorial Sloan-Kettering; human and mouse PSMA DNA vaccines (plasmid, prostate cancer), Memorial Sloan-Kettering; human papilloma virus vaccine, Transgene; HybriCell; hybrid cell vaccination, Humboldt University; HyperAcute vaccine (breast cancer), NewLink; HyperAcute vaccine (intradermal, prostate cancer), Newlink Genetics; HyperAcute vaccine (lung cancer), NewLink; HyperAcute vaccine (melanoma), Newlink Genetics; hypercalcemia vaccine (anti-PTH-rP, TDK), Immunovo; I i-key/MHC class II epitope hybrid peptide immunomodulator peptide vaccines (prostate cancer/colon cancer), Antigen Express; I i-key/MHC class II epitope hybrid peptide vaccines (HIV infection), Antigen Express; ibritumomab tiuxetan; ICT-107; ICT-111; ICT-121; ICT-140; IDD-1; IDD-3; IDD-5; idiotypic cancer vaccines, NCI/GTC Biotherapeutics; idiotypic vaccines, Biomira; IdioVax; IDM-2101; IDN-6439; IDO based cancer vaccine, Tectra; IEP-11; IGFBP-2 DNA plasmid vaccine (intradermal, ovarian cancer), Fred Hutchinson; IGN-101; IGN-201; IGN-301; IGN-311; IGN-402; IGN-501; Ii-key/MHC class II epitope hybrid peptide immunomodulator peptide vaccines (diabetes), Antigen Express; Ii-key/MHC class II epitope hybrid peptides (allergy), Antigen Express; IL-10 kinoid; IL-12 gene therapy, Baylor; IL-13, Sanofi; IL-15 smallpox vaccine, NCI; ILlaQb therapeutic vaccines (atherosclerosis), Cytos; IL-2 gene therapy (plasmid, transdermal, melanoma), Vical; IL-2 vaccine (gastric cancer), Newsummit; IL-2/CD40L-expressing leukemia vaccine, Baylor College of Medicine/MaxCyte; IL-2/CD80 expressing autologous whole cell vaccine (leukemia), King's College London; IL-4 gene therapy, Genetic Therapy/Univ Pittsburgh; IL-7/CD80-expressing allogeneic RCC-26 tumor cell vaccine (renal cell carcinoma), Charite-University Medicine Berlin; IMA-901; IMA-910; IMA-920; IMA-930; IMA-941; IMA-950; ImBryon; IMF-001; imiquimod; ImMucin; ImmuCyst; Immunecellgram; ImmuneFx; Immunodrug vaccines (CCR5) (HIV infection), Cytos; Immunodrug vaccines (HBV infection), Cytos; Immunodrug vaccines (osteoporosis), Cytos; Immunodrug vaccines (pancreatic/prostate cancer), Cytos; Immunodrug vaccines (vCJD), Cytos; Immunoglobulin G fusion proteins (melanoma), Wyeth; ImmunoVEX HSV2; IMO-2055; IMP-321; IMP-361; Imprime WGP; IMT-1012; IMT-504; IMVAMUNE; IMX-MC1; IMX-MEL1; inactivated bacterial vector vaccine (KBMA, HIV infection), Cents; inCVAX; IndiCancerVac; indinavir; INGN-225; INNO-305; INO-5150; Insegia; interferon alfa-2b; interferon-gamma gene therapy (cancer), Chiron/Cell Genesys; interleukin-12 cancer vaccine, University of Wisconsin; interleukin-lbeta, Celltech; interleukin-2 vaccine, ICR; inulin (gamma, ADVAX adjuvant), Vaxine; IPH-3102; IPH-3201; ipilimumab; ipilimumab+MDX-1379, Medarex/BMS; ipilimumab/IDD-1 combination vaccine (cancer), Medarex/IDM; IR-502; IRX-2; IRX-4; ISA-HPV-SLP; ISA-P53-01; ISCOMATRIX; ISS vaccine (cancer), Dynavax; Javelin—melanoma, Mojave; Javelin—papillomavirus, Mojave/Institut Pasteur; Javelin—prostate cancer, Mojave/Memorial Sloan-Kettering; JC virus vaccine (gastrointestinal cancer), Baylor Research Institute; JVRS-100; K562/GM-CSF; Kanda HPV Vaccine; KH-901; KLS-HPV; L19-IL-2 fusion protein, Philogen; L2 capsid protein-targeting monovalent vaccine (HPV), Advanced Cancer Therapeutics; L523S; labyrinthin vaccine (adenocarcinoma), ImmvaRx; lapuleucel-T; LewisY-KLH cancer vaccine, Memorial Sloan-Kettering; LG-768; LG-912; Lipomel; lipoprotein-based E6/E7 vaccines (HPV infection/cervical cancer), National Health Research Institutes; liposomal KSA vaccine, IDM Pharma; Lipovaxin-MM; liver cancer vaccine, Mayo Clinic; liver cancer vaccine, West Coast Biologicals; Lm Glioblastoma; Lm Melanoma; Lm Prostate; LMB-2; LMP-1/LMP-2 CTLs, Baylor College of Medicine/NCI; LN-020; LN-030; LN-040; LN-2200; lopinavir+ritonavir; Lovaxin M; Lovaxin NY; Lovaxin SCCE; Lovaxin T; LP-2307; LUD01-016; LungVax; Lx-TB-PstS1; lymphoma vaccine (ADX-40 adjuvant), Adjuvantix; M tuberculosis vaccine (LEAPS), CEL-SCI (MaxPharma); MAGE-3.A1 peptide (cancer), Ludwig Institute; MAGE-3-transduced autologous T-cell vaccine (anticancer), MolMed SpA/Takara Bio; malaria vaccine (LEAPS), MaxPharma/US Navy; MART-1 analogs, INSERM; Maxy-1200; mbGM-CSF tumor vaccines, IRC; MBT-2/VEGFR-2 RNA-transfected autologous dendritic cell vaccine (cancer), Duke University; MEDI-543; Melacine; Melan-A/IL-12, Genetics Institute; Melan-A/MART-1/ASO2B/Montanide ISA vaccine (melanoma), Ludwig/GlaxoSmithKline/Seppic; melanoma vaccine (ALVAC), Sanofi Pasteur; melanoma vaccine (GD3 ganglioside), Memorial Sloan-Kettering; melanoma vaccine (IMP-321, cancer), Immutep; melanoma vaccine (JuvaVax), Juvaris; melanoma vaccine (NA17.A2/tyrosinase/MART-1, gp100), Institut Curie; melanoma vaccine (pulsed antigen therapeutic), Metacine; melanoma vaccine (tyrosinase), Therion; melanoma vaccine (VRP), AlphaVax; melanoma vaccine, FIT Biotech; melanoma vaccine, Immunex; melanoma vaccine, Mayo Clinic/University of Leeds; melanoma vaccine, NYU; melanoma vaccine, PowderJect; melanoma vaccines, Novavax/National Cancer Institute; MelaVax; Melaxin; MelCancerVac; MEN-14358; MF-59; MGN-1601; MGV, Progenics; MicroPor (anticancer DNA vaccine), Altea; mifamurtide (liposomal), Millennium; milatuzumab-Fab-CEA-loaded dendritic cell vaccine (cancer), Immunomedics; MimoVac; MIS-416; MIS-416/immunogen (anthrax/malaria/tuberculosis/neutropenia), Innate; mitumomab; mitumprotimut-t; mixed vaccine (cancer), Zensun; MKC-1106-MT; MKC-1106-NS; MKC-1106-PP; ML-2400; MLCV liposome vaccine (B-cell lymphomas), Xeme; MMU-18006; mRNA transfected dendritic cell vaccine (melanoma/prostate cancer), GemVax; MTL-102; MTL-104; MUC-1 modulating plasmid DNA vaccine (transdermal patch formulation, ZP Patch technology, cancer), Zosano Pharma; MUC-1 naked cDNA vaccine (cancer), Cancer Research UK; MUC1 peptide vaccine program (cancer), UNMC; MUC1 targeted vaccine (cancer), Mayo Clinic; MUC-1 vaccine (pancreatic cancer), Corixa; MUC-1/CD40 cancer vaccine, Sidney Kimmel Cancer Center; MUC-1-KLH vaccine, Sloan-Kettering; MUC1-Poly-ICLC; MUC2-KLH conjugate vaccine (prostate cancer), Memorial Sloan-Kettering; multi-epitope peptide melanoma vaccine (MART-1, gp100, tyrosinase), University of Pittsburgh; multi-epitope tyrosinase/gp100 vaccine (melanoma), Memorial Sloan-Kettering; Multiferon; Multikine; multipeptide vaccine combination (melanoma), Ludwig; multivalent carbohydrate-based vaccine (cancer), Memorial Sloan-Kettering; multivalent HPV vaccine (CyaA), Genticel; mutant ras vaccine, NCI; MVA E2 vaccine (condyloma), Virolab/Universidad Nacional Autonoma De Mexico; MVA HER-2 AutoVac; MVA-BN-PRO; MVA-F6 vector (melanoma), Bavarian Nordic; M-Vax; MV-CEA; N-8295; NAcGM3/VSSP ISA-51 vaccine (cancer/HIV infection), Recombio/Center of Molecular immunology/Laboratorio; naked DNA (B-cell lymphoma) Vical/Stanford; necitumumab; nelfinavir; NeuroVax; NeuVax; Nfu-PA-D4-RNP; NGcGM3/VSSP (cancer), Recombio; NIC-002; NicVAX; non-Hodgkin lymphoma vaccine, Large Scale Biology; Norelin; NovoVAC-M1; NPC SAVINE (cDNA vaccine, nasopharyngeal carcinoma/EBV related lymphoma), Savine; NSC-710305; NSC-748933/OPT-821 vaccine; NTX-010; NV-1020; NY-ESO targeted vaccine (cancer), Dendreon; NY-E50-1 antigen, Genzyme Molecular; NY-E50-1 DNA vaccine (cancer), Ludwig Institute/PowderMed; NY-E50-1 vaccine (peptides), Ludwig Institute; NY-E50-1 vaccine (protein), Ludwig Institute; NY-E50-1/IL-12-expressing autologous lymphocytes (metastatic cancer), National Cancer Institute; OC-L vaccine (cancer), University of Pennsylvania; OCM-108; OCM-111; OCM-124; OCM-127; OCM-7342; OCV-101; OCV-105; OCV-501; ODC-0801; ODC-0901; OFA/iLRP-loaded autologous dendritic cell vaccine (breast cancer), Quantum Immunologics; oligodeoxynucleotides, Coley; Oligomodulators; oligonucleotide toll like receptor agonists (adjuvant, vaccination), Idera; OligoVax; OM-174; OM-197-MP-AC; OM-294-DP; Oncophage+co-adjuvant (cancer), Agenus/NewVac; OncoVAX, Vaccinogen; Oncovax-CL; OncoVax-P; ONT-10; ONY-P; Onyvax-105; Onyvax-CR; Onyvax-L; Onyvax-R; opsonokine tumor cell vaccine (GM-CSF/HA1), Genitrix; OpsoVac; OPT-822/OPT-821; oral vaccine (mucosal surface cancer), Kancer; oregovomab; OTSGC-A24; OV-2500; ovarian cancer vaccine (Listeria vector), Advaxis; O-Vax; P10s-Padre/Montanide ISA 51 vaccine (breast cancer), University of Arkansas; P16(37-63) peptide vaccine (HPV-associated cancer), Oryx GmbH; P-17; P-501; p53 cancer vaccine (canarypox vector, ALVAC), sanofi-aventis; p53 cancer vaccine, Virogenetics; PAGE-4 prostate cancer vaccine (PROVAX), IDEC; PankoPep; PankoVAC; PAP plasmid DNA vaccine (prostate cancer), University of Wisconsin-Madison; PAP vaccine, Hughes; papillomavirus vaccine (prophylactic), Large Scale Biology; PapViRx; PAS vaccine (GERD/pancreatic/colorectal/gastrointestinal cancers), Cancer Advances; PASD1 peptide DNA vaccine (cancer), CRT/University of Oxford/University of Southampton/King's College London; PBT-2; PDS-0101; PDS-0102; PE64-delta-553pi1; peginterferon alfa-2a; peginterferon alfa-2b; Pentarix; Pentrys; PEP-223/CoVaccine HT; peptide vaccine (cancer), VaxOnco; peptide vaccine (glioma), University of Pittsburgh; peptide vaccine (hepatocellular carcinoma), OncoTherapy/Ono; peptide vaccines (colon cancer), OncoTherapy/Otsuka; peptide-based targeted vaccines (cancer/infectious disease, DNL/HLA-antibody), Immunomedics/Alexis; peptide-based vaccines, BTGC/Yeda; peptide-GM-CSF/IL-2 vaccination therapy, Univ South Carolina; personalized cancer vaccine (autologous hemoderivative), PharmaBlood; personalized peptide vaccine (anticancer), Green Peptide; personalized recombinant protein vaccines (cancer), Genitope; PEV-6; pexastimogene devacirepvec; PN-2300; pNGVL-4a-CRT/E7 (detox) DNA vaccine (TriGrid/im, cancer), Ichor Medical Systems; POL-103A; Poly-ICLC; poly-ICLC adjuvanted vaccines (cancer), Oncovir; Polynoma-1; Polyshed-1; polysialic acid/KLH/QS-21 vaccine, Memorial Sloan-Kettering; polyvalent prophylactic vaccine (melanoma), MabVax; polyvalent prophylactic vaccine (neuroblastoma), MabVax; pox virus B7.1 cancer vaccine, Therion Biologics/NCI; pox virus CD40L vaccine (lymphoma), Therion; PR1 peptide antigen vaccine (leukemia), Vaccine Company; pradefovir; PRAME-SLP; Procervix; progenipoietin G; prophylactic vaccine (HPV infection), Dynavax; prostate cancer vaccine (IMP-321), Immutep; prostate cancer vaccine (VSV vector transduced with prostate cDNA), Mayo Foundation; prostate cancer vaccine, FK Biotecnologia; prostate cancer vaccine, Oncbiomune; prostate cancer vaccine, United Biomedical; protein subunit vaccine (lung cancer), MUbio Products BV; protein vaccines (Targosphere, malaria/cancer), Rodos BioTarget; PRO-VAX (anticancer), Shanghai Genomics; PRX-302; PS-2100; PSA RNA transfected autologous dendritic cell vaccine (cancer), Duke University; Pseudomonas aeruginosa vaccine (oral), Provalis; PSMA pharmaccine, Pharmexa; PSMA subunit vaccine (prostate cancer), Progenics/CYTOGEN; PSMA-ADC; PSMA-VRP; pSP-D-CD40L; pSP-D-GITRL; PT-107; PT-123; PT-128; PT-207; PVAC; PVX-410; QS-21; racotumomab; ranagengliotucel-T; RANKL AutoVac; recombinant HPV-16 VLP vaccine, Novavax/NCI; recombinant human Erbb3 fragment therapeutic tumor vaccine (injectable, Erbb2-overexpressing cancer), Zensun; recombinant pox virus vaccine (gp100, melanoma), NCI; recombinant pox virus vaccine (her2/neu, breast cancer), Therion; recombinant pox virus vaccine (MAGE-1), Therion/Aventis Pasteur; recombinant pox virus vaccine (MART-1), Therion/Aventis Pasteur; recombinant prolactin, Genzyme; recombinant protein based vaccine (cervical cancer/HPV infection), Antagen Biosciences; recombinant vaccine (colon cancer), National Institutes of Health; recombinant vaccinia virus vaccine (MUC-1), Therion; Reniale; resiquimod (topical), 3M/Celldex; RetroVax-MAGE-3; rhCMV-based vector vaccine program (cancer), Virogenomics; rindopepimut; rintatolimod; RN-2500; RNF43-721; Roferon-A; RPK-739; rV-CEA-TRICOM+rF-CEA-TRICOM prime-boost colorectal cancer vaccine, Therion; rV-NY-ESO-1/rF-NY-ESO-1 prime-boost breast cancer vaccine, Ludwig Institute/Therion; rV-PSA+rilimogene glafolivec prime-boost prostate cancer vaccine, Therion; rV-PSA-TRICOM/rF-PSA-TRICOM prime-boost prostate cancer vaccine, NCI/BN ImmunoTherapeutics; S-288310; S-488210; S-488410; sargramostim; SART3 peptide cancer vaccine, Kurume University; SCIB-1; SCIB-2; SD-101; SDZ-SCV-106; seasonal influenza vaccine (VLP), Novavax/Cadila; semi-allogenic vaccines (cancer), SemiAlloGen; SFVeE6,7; SGD-2083; sialyl Lea-KLH conjugate vaccine (breast cancer), MabVax; Simplirix; sipuleucel-T; SL-701; sLea-KLH vaccine (cancer), Optimer Therapeutics; SLP vaccine (cervical cancer), Leiden University Medical Center; SP-1017; SRL-172; SSS-08; stage IV melanoma vaccine, Adelaide Research & Innovation; stem cell therapy (HIV), Targeted Genetics/Hutchinson Center/Genetics Therapy; stress gene therapy (cancer), Stress/Genzyme LCC; STxB-E7; suicide gene therapy (HSV-TK), Tulane/Schering-Plough; survivin/midkine vaccine (cancer), Vaxeal; SV-BR-1-GM; synthetic long peptide based vaccines against antigen X/Y (cancer), ISA Pharmaceuticals; T1-IR; TAAVac (cancer), Genticel; TA-CIN; TA-GW; TA-HPV; talimogene laherparepvec; TAP-1 gene therapy, TapImmune; targeted CTLs (CMV), Targeted Genetics; TARP peptide vaccines (prostate cancer), NCI; TARP peptide-pulsed autologous dendritic cell vaccine (prostate cancer), NCI; tasonermin; TBI-4000; technetium Tc 99m etarfolatide; TEIPP-01; TeloB-VAX; telomerase-targeted vaccine (cancer), Dendreon; telomerase-transduced autologous lymphocytes (cancer), Cosmo Bioscience; tertomotide; TG-01, Targovax; TG-1024; TG-1031; TG-1042; TG-4010; TG-4040; TGF beta kinoid; TGF-alpha vaccine (cancer), Bioven Holdings/CIMAB; TGFB2-antisense-GMCSF vaccine (cancer), Gradalis; Theradigm-CEA; Theradigm-Her-2; Theradigm-p53; Theradigm-prostate; Theramide; therapeutic cancer vaccine (human papillomavirus infection), Okairos; therapeutic cancer vaccine (synthetic antigen mimetic/virus-like particle), Virometix; therapeutic cancer vaccines (VLP), Redbiotec; therapeutic cancer vaccines, Circadian/Monash; therapeutic multiepitope vaccine (LT-fused, melanoma), Dan Immunotherapy; therapeutic peptide subunit vaccine (prostate cancer), CIGB; therapeutic vaccine (glioma), Epitopoietic Research; therapeutic vaccine (oral, colon cancer), Bio-Bridge; Theratope; thymalfasin; tipapkinogene sovacivec; TLR-7/TLR-8 agonists (cancer), Pfizer; TMX-202; TNF alpha kinoid; TNF-alpha AutoVac, Pharmexa; tobacco plant-derived anti-idiotype vaccine (subcutaneous magnICON, non-Hodgkin's lymphoma), Bayer/Icon; Tolamba; total tumor RNA transfected autologous dendritic cell vaccine (cancer), Duke University; trametinib DMSO; TRC-105; tremelimumab; TriAb; TriGem; trimelan (ImmunoVEX, melanoma), BioVex; TroVax; TroVax-DC; TRP-1 protein vaccine (melanoma), ImClone; TRP-1/TRP-2, NIH; TRP-2 peptide-based therapeutic cancer vaccine (Vaxfectin), Vical; TRP2-electroporated autologous dendritic cell therapy (melanoma), Memorial Sloan-Kettering; TRX-385; TRX-518; TSD-0014; tucaresol; tucotuzumab celmoleukin; tumor cell vaccines (melanoma), Centro de Investigaciones Oncologicas; tumor vaccine, University of Pittsburgh; tumor-antigen-specific lymphocytes, Corixa; tumor-associated carbohydrate epitope mucin vaccine (cancer), Recopharma; tumor-specific dendritic cell vaccines (cancer), BioPulse; UltraCD40L; UltraGITRL; umbilical cord stem cell therapy (hematological cancer), Novartis; UniDC program; unspecified autologous immunotherapeutic product (cancer), Personal Biotechnology; uPA-targeted interferon-beta-expressing oncolytic Sendai virus (cancer), DNAVEC/Kyushu University; V-212; V-502; V-503; V-934/V-935; vaccine (3H1 mAb), Kentucky Uni; vaccine (anticancer), Norsk Hydro; vaccine (B cell lymphoma), Immune Response Corp; vaccine (B-cell lymphoma), University of Connecticut; vaccine (cancer) (1), Immunomedics; vaccine (cancer) (2), Immunomedics; vaccine (cancer), Biochem Pharma; vaccine (cancer), Genzyme Molecular Oncology; vaccine (cancer), Intercell; vaccine (cancer), Jenner/Walter Reed; vaccine (cancer), Sandoz/Wistar; vaccine (cancer), University of Alberta/Briana; vaccine (cervical cancer), Johns Hopkins; vaccine (colorectal tumor), Therion/Aventis Pasteur; vaccine (EBV), BioResearch Ireland; vaccine (gastrointestinal cancers), Astrimmune; vaccine (GI tumor), Wistar; vaccine (Her-2/neu), Corixa/GlaxoSmithKline; vaccine (human papilloma virus), Chiron; vaccine (melanoma), Dana-Farber; vaccine (melanoma), Genzyme Molecular/NCI; vaccine (melanoma), Pevion; vaccine (melanoma), University of Virginia; vaccine (multidrug resistant cancer), AC Immune; vaccine (naked DNA, HBV), Merck & Co; vaccine (naked DNA, HPV), Vical; vaccine (naked DNA, HSV), Vical; vaccine (naked DNA, influenza), Vical; vaccine (naked DNA, prostate cancer), Vical; vaccine (naked DNA, TB), Merck & Co; vaccine (non-Hodgkin's lymphoma), Malaghan Institute of Medical Research; vaccine (pentavalent, small-cell lung cancer), Memorial Sloan-Kettering; vaccine (prostate tumor), Corixa/SB Biologicals; vaccine (ras protein), IDEC; vaccine (tetravalent, small-cell lung cancer), MabVax; vaccine (tuberculosis), StressGen; vaccine targeting midkine (cancer), Vaxeal; vaccine targeting survivin (cancer), Vaxeal; vaccines (HI-557 technology, viral infection/bacterial infection/cancer), Bioxyne; vaccines (Immunobody, colorectal cancer), Scancell/immatics; vaccines (nanoparticle formulation, infection/metabolic disorder/CNS disease/cancer), Selecta Biosciences; vaccinia virus therapy, Thomas Jefferson; vaccinia/fowl pox TRICOM vaccine (cancer), Therion; vadimezan; VB-1014; Vbx-011; Vbx-016; Vbx-021; Vbx-026; VEGF kinoid; VEGF vaccine, Protherics; VEGFR1-770NEGFR1-1084 peptide vaccines (renal cell carcinoma), Kinki University/Tokyo University; Veldona; velimogene aliplasmid; venom peptide-based cancer vaccine, Canopus; vesicular stomatitis virus vector recombinant vaccine (cancer), University of Leeds; VG-LC; VGX-3100; VGX-3200; VIR-501; viral fusogenic membrane glycoproteins, Mayo/Cambridge Genetics; vitalethine; vitespen; VLI-02A; VLI-02B; VLI-03B; VM-206; VPM-4001; VSV-G vaccine, Mayo/ICRF; Vx-001; Vx-006; VX-026; VX-25; Vxb-025; Vxb-027; VXM-01; whole cell vaccine (intradermal, breast cancer), Oncbiomune; WT1 peptide vaccine (cancer), Charity Medical School of the Humboldt University of Berlin; WT1 peptide-based cancer vaccine, Japan National Cancer Research Center; WT1 protein-based vaccine (leukemia/lymphoma), Corixa; WT1-dendritic cell vaccine (hematological cancer), National Institutes of Health; WT1-targeted autologous dendritic cell vaccine (cancer), University of Antwerp; WT-4869; XToll; ZFP TF (GM-CSF upregulator, cancer), Sangamo/Onyx; zona pellucida antigens (ovarian cancer), Pantarhei Bioscience BV; and the like.

The therapeutic agent can be a radioactive material. Suitable radioactive materials include, for example, of ⁹⁰yttrium, ¹⁹²fridium, ¹⁹8gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ¹²³lead ¹¹¹Indium, ⁵²iron, ¹⁶⁷thulium, ⁵⁷nickel, ⁶²zinc, ⁶²copper, ²⁰¹thallium and ¹²³iodine. Without wishing to be bound by a theory, aggregates comprising a radioactive material can be used to treat diseased tissue such as tumors, arteriovenous malformations, and the like.

The aggregates, red blood cells and microcapsules described herein can be used as in vivo imaging agents of tissues and organs in various biomedical applications including, but not limited to, imaging of blood vessel occlusions, tumors, tomographic imaging of organs, monitoring of organ functions, coronary angiography, fluorescence endoscopy, laser guided surgery, photoacoustic and sonofluorescence methods, and the like. The aggregates, red blood cells and microcapsules described herein are useful for detection and/or diagnosis of atherosclerotic plaques, or blood clots. When used in imaging applications, the aggregates, red blood cells and microcapsules described herein typically comprise an imaging agent, which can be covalently or noncovalently attached to the aggregate.

Accordingly, in some embodiments, the compound is an imaging agent or contrast agent. As used herein, the term “imaging agent” refers to an element or functional group in a molecule that allows for the detection, imaging, and/or monitoring of the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. As used herein the term “contrast agent” refers to any molecule that changes the optical properties of tissue or organ containing the molecule. Optical properties that can be changed include, but are not limited to, absorbance, reflectance, fluorescence, birefringence, optical scattering and the like.

In some embodiments, the compound is a diagnostic reagent.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H, 5H, 11H, 15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; and the like. Many suitable forms of these fluorescent compounds are available and can be used. In some embodiments, the imaging or contrast agent is a coumarin.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³, ¹²⁴I, and ¹²⁵I.

Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd.

Suitable paramagnetic metal ions include, but are not limited to, Gd(III), Dy(III), Fe(III), and Mn(II).

Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide is bound to a chelating agent or chelating agent-linker attached to the aggregate. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the nanoparticles.

In some embodiments, the imaging agent can be selected from the group consisting of [111In]B3; [111In]SRVII23; [124I]DIATHIS-1; [18F]-AH113804; [18F]DCFPyL; [18F]ICF-01006; [99mTc]Met; 105A5; 111In antisense oligonucleotide CDK inhibitor imaging agent (intravenous, Cancer), University of Toronto; 111In anti-tPA, Novo Nordisk; 111In RM-2; 111In-Benzyl-DTPA-Z(HER2:342)-pep2; 111In-capromab pendetide; 111In-GLP-1 analogs (neuroendocrine tumor imaging); 111In-labeled lactam bridge-cyclized alpha-melanocyte-stimulating hormone peptide (melanoma), NuView/University of New Mexico; 111In-labeled LFA-1 targeted imaging agent (lymphoma/leukemia), NuView/University of New Mexico; 11C-6-Me-BTA-1; 11C-atrasentan PET imaging agent (cancer), Abbott; 11C-AZD-2184; 11C-AZD-2995; 11C-carfentanil; 11C-GSK-215083; 11C-labeled sigma opioid receptor ligands, Santen; 11C-LY-2795050; 11C-MePPEP; 11C-MICA; 11C-MK-3168; 11C-MK-8278; 11C-PBR-170; 11C-PBR-28; 11C-R-129144; 11C-RU-40555; 123I-CMICE-013; 123I-DRM-106; 123I-eptacog alfa (bleeding), Novo Nordisk; 123I-IMPY; 123I-iodometomidate; 123I-iofetamine; 123I-ioflupane; 123I-iomazenil, Nihon Medi-Physics; 123I-iometopane; 123I-labeled dopamine antagonist (Parkinsonistic features), Copenhagen University; 123I-MIBG, Molecular Insight; 123I-MNI-168; 123I-MNI-330; 123I-MNI-420; 123-iodine labeled exendin derivatives (imaging GLP-1 receptors, diabetes), Kyoto University/Arkray; 123I-TM-601; 124I-A33; 124I-labeled 11-1F4; 124-iodine-labeled PSCA targeting minibody (cancer), ImaginAb; 124I-PGN-650; 125I-AnnA1 IgG; 125I-MIBG, Neoprobe/Childrens Cancer Group/CIS; 125-Iodine-labeled MFE-23; 131I-chTNT-1/B; 131I-radretumab; 131I-TM-601; 177Lu-AMBA; 178Tantalum; 18F ISO-1; 18F labeled ethanolamine derivatives (cancer imaging), Bayer Schering; 18F-AV-45 dimer; 18F-BAY-85-8050; 18F-FDDNP; 18F-FEDAA-1106; 18F-FEPPA; 18F-fluoromethylallylcholine; 18F-flutabine; 18F-F-PEB; 18F-FRP-170; 18F-labeled fluoropolyethylene glycol derivatives (Alzheimers disease detection), University of Pennsylvania; 18F-labeled glyburide analogs, University of Pennsylvania; 18F-labeled nAChR antagonists (Alzheimers disease), University of California Irvine; 18F-labeled PET imaging agent (melanoma), Wake Forest University; 18F-MNI-558; 18F-NST-ML-10; 18F-SKI-696; 18F-SMIBR-K5; 18F-SMIBR-W372; 18F-VEGF binding peptides (PET imaging), Genentech; 203Pb/212Pb-radiolabled ErbB-2 receptor targeting peptides (cancer), AlphaMed; 227Th-rituximab (cancer), Algeta; 28A32; 3E8; 5-aminolevulinic acid hydrochloride (glioma imaging), Nobelpharma; 62Cu-ATSM; 62Cu-ETS; 62Cu-PTSM; 64Cu-AMG-655; 64Cu-TM-601; 64-Cu-TP-3805; 68Ga-based PET tracer (cancer imaging), Novo; 68Ga-EC-G; 6-FPOL; 76Br-16alpha,17alpha-dioxolane progestin analogs (breast cancer), Washington University/University of Illinois; 98mTC-CIM-ANT; 99mTc-betafectin; 99m-Tc labelled annexin V-128 (rheumatoid arthritis/Crohn's disease), Atreus; 99m-Tc MAG3-HER2/MUC1 peptide (breast cancer), King Faisal; 99mTc TR-21; 99mTc-anti-ED-B; 99mTc-AP(4)A; 99mTc-apcitide injection; 99mTc-besilesomab; 99mTc-ciprofloxacin, DRAXIS; 99mTc-ciprofloxacin, INMAS; 99mTc-Demogastrin 2 (medullary thyroid cancer), Biomedica Life Sciences; 99mTc-depreotide; 99mTc-DTPA; 99mTc-DTPA-Glipizide; 99mTc-EC-0652; 99mTc-EC-DG; 99mTc-EC-metronidazole; 99mTc-fanolesomab; 99mTc-glucarate; 99mTc-Hynic-Annexin V; 99mTc-labeled non-steroidal analogs (cancer, imaging/detection), Roche; 99mTc-labeled PSMA inhibitors (prostate cancer, imaging), Johns Hopkins University; 99mTc-labelled adrenomedullin (pulmonary disease), PulmoScience; 99mTc-maraciclatide; 99mTc-MAS3-TM-601; 99mTc-MIP-1340; 99mTc-MIP-1404; 99mTc-MIP-1405; 99mTc-MIP-1407; 99mTc-MSA; 99mTc-N4-Tyrosine; 99mTC-NC-100668; 99mTc-N-DBODC5; 99mTc-nitrocade; 99mTc-nitroimidazole, Bristol-Myers Squibb; 99mTc-P215; 99mTc-P424; 99mTc-P483H; 99mTc-P587; 99mTc-P748; 99mTc-rBitistatin; 99mTc-rotenone conjugates (cardiac perfusion), Molecular Insight; 99mTc-RP-128; 99mTc-seglitide analog, DRAXIMAGE; 99mTc-sestamibi; 99mTc-siboroxime; 99mTc-sulesomab; 99mTc-teboroxime; 99mTc-tetrofosmin; 99mTc-TP-850; 99m-Tc-tropantiol; 99m-Technetium labeled azetidinylmethoxypyridine derivatives (nervous system imaging), Kyoto University; A-84543; AB-3025-11; ABD-035; Abdoscan; ABY-025; ABY-026; ABY-028; acetylcholinesterase (AChE) inhibitors (Alzheimer's disease), University of California/Scripps Institute/Siemens Medical Solutions Molecular Imaging; Adenoscan; AdreView; AGT-100; AGT-160; Albunex; alpha-7 nicotinic receptor binding PET ligands (neurological disorders), NeuroSearch/University of Copenhagen; Altropane; AMI-121; AMI-25; AMI-HS; amyloid beta MRI contrast agents (Alzheimers), Mayo Clinic; amyloid beta oligomers (imaging agent), University of California Davis; amyloid binding PET ligands (Alzheimers disease), Aventis; ANA-5 analog (oral radiolabelled imaging agent, Alzheimers disease), Alzhyme; androgen receptor modulators (imaging, cancer) University of Nebraska Medical Center; anti PSA antibody conjugates (prostate cancer therapy/diagnosis), Molecular Imaging and Therapeutics; antibodies conjugated fluorochromes/radionuclides (cancer), TTFactor srl; antimelanoma antibodies, MabCure; Anti-ZnT8 antibody imaging agent (diabetes), Mellitech SAS; AP-2011; apadenoson; arcitumomab; AT-004; atrial natriuretic peptide, DRAXIMAGE; AVP-4; AVP-5; AVP-6; AVP-7; AZD-4694; Azedra; AZPET; BAY-1006451; BAY-1006578; BAY-1075553; BAY-1163615; BAY-85-8102; BAY-86-4367; BAY-86-4884; BAY-86-7548; BAY-86-9596; BChE inhibitors (imaging, Alzheimers disease), University of Nebraska Medical Center; BCI-632; beta1-adrenoceptor-targeted imaging agents (cardiovascular disease), Lantheus; BFPET; binodenoson; bivalirudin (nanoparticle, thrombosis), Kereos; BMIPP, Nihon; BMS-753951; BOT-502; BR-14; BR-55; BT-19; BT-20; BT-23; BW-42; BY-963; C11-SB-207145; calcium nanoparticles (cancer detection), BioLink; cancer imaging agent, AltaRex/Resolution Pharm; cancer imaging agents, MallincKrodt/Optimedx; Capiscint; carbonic anhydrase IX inhibitors (cancer, imaging), Molecular Insight; carborane-containing arylphosphonium salts (imaging/boron neutron capture therapy, cancer), University of Sydney; cardiac imaging agents (ACE targeting), Molecular Insight/University of Maryland; CardioPET; Cavisomes; CB1 antagonists (brain imaging), Johns Hopkins; cell penetrating peptide (diagnostic, cancer), CDG; CEN-109; CGRP-A2 radioligand agent (migraine), Merck; chlorin-e6-conjugated mucin-targeted aptamers (photodynamic therapy/imaging, cancer), Ontario Cancer Institute; CLR-1404 (fluorescent analogs); CMC-001; CMUS-100; CNS-1261; cocaine analogs, Indiana University; Collagelin; CTP, Hafslund Nycomed; CTT-54; Cu64-CND1-PNA; Cu64-CNND1-B; Cu64-CNND1-L; CUSCA; D-04; Demobesin; depelestat; diagnostic agent (infectious diseases), Univalor; DMP-444; DOTA-BASS (cancer), Salk Institute; DOTA-NT-MSH targeted alpha particle-emitting radionuclides (cancer), AlphaMed; DX-182; E-7210; EchoGen; Echovist; EM-2198; EM-3106B; ENDG-4010; EP-1242; EP-1873; EP-2104R; EP-3533; EP-862; EPI-HNE-2; EVP-1001-1; eye disease program, NuvOx Pharma; F-18 exendin-4 derivative PET tracers (diabetes), Kyoto University/Arkray; F-18-CCR1; F-18-HX4; F-18-VM4-037; FerriSeltz; ferumoxtran-10; ferumoxytol; fibrin-binding radiodiagnostic (thrombosis), DRAXIMAGE/Savient; florbenazine (18F); florbetaben (18F); florbetapir (18F); florilglutamic acid (18F); fluciclatide F 18; Fluoratec; fluorescein derivative contrast agent (imaging, ocular disease), Philogen; fluorescent LYVE-1 antibody (imaging agent, cancer), University of California/Anticancer Inc; fluorine-18-based PET imaging agents (neuropsychiatric disorders), Janssen; fluorine-18-labelled peptides (PET cancer imaging), Immunomedics; fluoropegylated indolylphenylacetylenes (Alzheimer's disease), Avid; flurpiridaz F 18; flutemetamol (18F); folate-targeted imaging agents (inflammation), Endocyte/Purdue University; fullerene-encapsulated MRI imaging agents, Luna Innovations; functionalized liposomes (stroke), Universidade de Santiago de Compostela; gadobenic acid; gadobutrol; gadocoletic acid; gadodiamide; gadofluorine 8; gadofosveset; gadolinium based C60 fullerene-paclitaxel-ZME-018 conjugates (prodrug/imaging, cancer), TDA Research/Rice University/MD Anderson; gadolinium texaphyrin; gadolinium texaphyrins (imaging, atherosclerosis), Pharmacyclics; gadolinium zeolite; gadomelitol; Gadomer-17; gadopenamide; gadopentetate dimeglumine; gadoteridol; gadoversetamide; gadoxetate disodium; gallium-68 pasireotide tetraxetan; Gd contrast agents (liposomal nanoparticles), ImuThes Therapeutics; GE-226; Glio-Image, Targepeutics; Gliolan; GL-ONC1; GlucaGen; GlucoMedix; Glysopep; GlyT1 PET radiotracers (schizophrenia), Merck & Co; GN-1140; GP-2-193; GTx-100; GW-7845; hedgehog labelled stem cells (cancer), Radiomedix; Hexvix; hMAG-1 targeting GRSA (imaging, breast cancer), Woomera; HRC-201; humanized ATA antibodies (imaging, cancer), Enlyton; humanized mAbs (breast cancer), Kalgene; HumaSPECT; hyaluronic acid-Gd, Hyal; I-124-CLR1404; ibritumomab tiuxetan; IL-8 analogs, Diatech; imaging agent (infectious disease), NuView; imaging agent (pancreatic cancer), NuView/University of New Mexico; imaging-theranostic nanoemulsion agents (multidrug resistant ovarian cancer), Nemucore/Fox Chase Cancer Center/Northeastern University; IN-N01-OX2; INP-04; intetumumab; iobitridol; iodine (124I) girentuximab; iodine-124-labeled F-16 scFv antibody (PET immunodetection, cancer), Philogen; iodixanol; iodofiltic acid (123 I); ioflubenzamide (131I); iofolastat I 123; ioforminol; iohexol; iomeprol; iopamidol; iopiperidol; iopromide; iosimenol; iosimide; iotrolan (oral, X-ray imaging), Schering AG; J-001X; KDF-07002; KI-0001; KI-0002; KI-0003; KI-100X; labeled TSH superagonists (thyroid cancer), Trophogen; landiolol (coronary imaging), Ono; LeucoTect; Levovist; LipoRed; LM-4777; LMI-1195; Lumacan; LumenHance; LymphoScan; mangafodipir; matrix metalloproteinase inhibitor (atherosclerosis), Lantheus; MB-840; meglumine gadoterate; Metascan; mGlu2 receptor PET ligand (psychiatric disease), Johnson & Johnson; mGluR5 PET tracers (neurodegenerative disease), Merck & Co; MH-1, American Biogenetic; MIP-160; MIP-170D; MIP-170S; MM-Q01; MN-2011; MN-3015; Monopharm-C; MRX-408; MRX-825; MS-136; MS-264; myocardial imaging agent, Mallinckrodt; Myomap; N-0861; N-1177-inh; N-1177-iv; N-1177-sq; nAChR PET agent, NIDA; NanoBarium; NanoLymph; nanoparticle MRI agents (Alzheimers disease/cancer), Senior Scientific; nanotherapeutics (breast cancer, lung cancer, infectious diseases, sepsis, atherosclerosis), SignaBlok; NC-100150; NC-100182; NCL-124; NCTX; NK3 antagonist PET ligand (psychiatric disease), AstraZeneca; NMDA radioligands, Kyushu University; NMK-36; nociceptin/orphanin FQ receptor PET ligands (neuropsychiatric disorders), Eli Lilly; nofetumomab; NP-50511; NS-2381; NSI-1; NVLS/FMAU; NVLS/FX-18A; OBP-401; octafluoropropane; OctreoScan; oligonucleotide (HNE), NeXstar; omacianine; Oncotec; Oralex; OvaFluor; oxidronic acid; oxilan; P-3378; P-773; P-947; PB-127; Pb-203 labeled [DOTA]-ReCCMSH targeted alpha particle-emitting radionuclides (cancer), AlphaMed/University of Missouri; PCP-Scan; PDL-506; Pentacea; Pepscan; peptide-based PET radiotracer (breast cancer), Stanford University Medical Center; perflexane-lipid microsphere; perflubutane (lipid microsphere-encapsulated, imaging), Daiichi Sankyo; perflubutane (polymer microsphere-encapsulated, heart disease), Acusphere; perflutren lipid microsphere; PET imaging agent (Alzheimer's disease), AC Immune; PET imaging agent (anti-5T4 tumor antigen Ab, ovarian cancer), ImaginAb; PET imaging agent (cancer), Cancer Targeted Technology/Bayer; PET imaging agent (melanoma), Acaduceus; PET imaging agent (neurodegenerative diseases), Fujisawa; PET imaging agent (thrombosis), Astellas; PET imaging agents (cancer), Affinity Pharmaceuticals; PET imaging agents (cardiovascular disease), ImaginAb/GE Healthcare; PET radiotracer (prostate cancer), Johns Hopkins University School of Medicine; PET radiotracer (solid tumors), MD Anderson Cancer Center; phosphodiesterase 10 imaging agent (PET, neurological disorders), Institute for Neurodegenerative Disorders; PIMBA; Prognox; ProScan-A; ProstaFluor; ProstaLite; Prostatec; Prostaview; PT-16; pyridyl benzofuran derived imaging agent (nervous system disorder), Kyoto University; Quantison; QW-7437; radiolabeled antibodies, University of Sydney/ANSTO; radiolabeled anti-CD4 monoclonal antibody fragment (imaging agent, chronic inflammation), Biotectid; radiolabeled anti-CEACAM6 antibodies (imaging/cancer), NIH; radiolabeled anti-PSMA huJ591 minibodies (prostate cancer), ImaginAb; radiolabeled anti-RECAF antibodies (cancer), BioCurex; radiolabeled DTPA-adenosylcobalamin, Copharos; radiolabeled HPMA copolymer conjugates (angiogenesis), Molecular Insight; radiolabeled iodobenzamide, INSERM; radiolabeled leukotrine B4 antagonist, University of Nijmegen/BMS; radiolabeled onartuzumab (imaging, cancer), Genentech; radiolabeled sigma-2 receptor ligands (solid tumor), Washington University in St Louis; radiolabeled VEGF (cancer), Sibtech/Stanford; radiolabeled VEGFR-1 inhibitors (cancer), IASON; radiolabeled WC-10 (neurological disease), Washington University; radiolabelled-A20FMDV2; radiotargeted gene therapy HSV1-tk (cancer), KIRAMS; recombinant TSH superagonists (thyroid cancer), Trophogen; regadenoson; RESP-3000; RG-7334; RP-431; RP-517; RP-748; samarium-153-DOTMP; SapC-DOPS, Molecular Targeting Technology/Bexion; secretin human; seprase inhibitors (cancer, imaging), Molecular Insight; SF-25; SH-U-555-C; SH-U-563; sigma-opioid ligand, NIH; SLX-1016; somatostatin analogs, Neoprobe; SonoRx; SPAGO Pix; SPIO-Stasix nanoparticles (imaging/therapeutic, prostate cancer), Androbiosys/Roswell Park Cancer Institute; sprodiamide; SPVF-2801-10; SR-4554; STARBURST dendrimer-based MRI contrast agents (cardiovascular disease/ovary cancer), Dendritic Nanotechnology; steroid mimics (breast cancer imaging/therapy), Daya Drug Discoveries; sulphur hexafluoride microbubble ultrasound agent, Bracco; targeted nanoparticle-enhanced pro-apoptotic peptides (glioblastoma), Sanford-Burnham/Salk Institute; targeted two-photon photodynamic therapy (cancer), SensoPath; tau-binding PET tracer (Alzheimer disease), Siemens; Tc99-labeled 14F7 humanized mAb (cancer imaging), The Center of Molecular Immunology; T-cell co-receptor targeting PET imaging agent (antibody fragment, cancer/inflammation/transplantation), ImaginAb; Tc-HL-91; TechneScan Q12; technetium (99m Tc) bicisate; technetium Tc 99m etarfolatide; technetium Tc 99m tilmanocept; technetium-99m-RP-414, Resolution; TF-12-radiolabeled IMP-288 (cancer), Immunomedics; TF-2 plus diagnostic/therapeutic (cancer), Immunomedics; Tin-117m-labeled annexin (heart disease), Clear Vascular; TKS-040; TLC 1-16; TomoRx; TPM+imaging agents; transcript imaging technology, Sugen/NCI; TRC-105; triiodobenzene contrast agents, Nycomed; Tru-Scint; TSARs, Cytogen/Elan; tumor endothelial marker antibodies (anticancer), Genzyme/John Hopkins; undisclosed compounds (epithelial/thyroid cancer), Kalgene; VasoPET; VEGF superagonists (neovascularization), Trophogen; ViaScint; VINP-28; VK-11; VMAT2 ligands (CNS disorder imaging), Molecular Neurolmaging/Institute for Neurodegenerative Disorders; WIN-70197; yttrium (90Y) clivatuzumab tetraxetan; Zn-DPA-B; Zn-DPA-G; Zn-DPA-H; Zn-DPA-I; Zn-DPA-P; and any combinations thereof.

In some embodiments, the contrast agent can be selected from the group consisting of [111In]SRVII23; [124I]DIATHIS-1; [18F]-AH113804; [18F]DCFPyL; 111In RM-2; 111In-Benzyl-DTPA-Z(HER2:342)-pep2; 11C-6-Me-BTA-1; 11C-atrasentan PET imaging agent (cancer), Abbott; 11C-AZD-2184; 11C-AZD-2995; 11C-carfentanil; 11C-GSK-215083; 11C-labeled sigma opioid receptor ligands, Santen; 11C-LY-2795050; 11C-MePPEP; 11C-MICA; 11C-MK-3168; 11C-MK-8278; 11C-PBR-170; 11C-PBR-28; 11C-R-129144; 11C-RU-40555; 123I-DRM-106; 123I-IMPY; 123I-iofetamine; 123I-iometopane; 123I-MIBG, Molecular Insight; 123I-MNI-168; 123I-MNI-420; 123-iodine labeled exendin derivatives (imaging GLP-1 receptors, diabetes), Kyoto University/Arkray; 124I-labeled 11-1F4; 131I-chTNT-1/B; 131I-radretumab; 18F ISO-1; 18F labeled ethanolamine derivatives (cancer imaging), Bayer Schering; 18F-AV-45 dimer; 18F-BAY-85-8050; 18F-FDDNP; 18F-FEDAA-1106; 18F-FEPPA; 18F-fluoromethylallylcholine; 18F-F-PEB; 18F-labeled fluoropolyethylene glycol derivatives (Alzheimers disease detection), University of Pennsylvania; 18F-labeled glyburide analogs, University of Pennsylvania; 18F-labeled nAChR antagonists (Alzheimers disease), University of California Irvine; 18F-labeled PET imaging agent (melanoma), Wake Forest University; 18F-MNI-558; 18F-NST-ML-10; 18F-SKI-696; 18F-SMIBR-K5; 18F-SMIBR-W372; 18F-VEGF binding peptides (PET imaging), Genentech; 62Cu-ATSM; 62Cu-ETS; 62Cu-PTSM; 64Cu-AMG-655; 64-Cu-TP-3805; 68Ga-EC-G; 76Br-16alpha,17alpha-dioxolane progestin analogs (breast cancer), Washington University/University of Illinois; 99mTc TR-21; 99mTc-anti-ED-B; 99mTc-EC-DG; 99mTc-labeled PSMA inhibitors (prostate cancer, imaging), Johns Hopkins University; 99mTc-maraciclatide; 99mTc-MAS3-TM-601; 99mTc-teboroxime; 99m-Tc-tropantiol; A-84543; AdreView; Albunex; alpha-7 nicotinic receptor binding PET ligands (neurological disorders), NeuroSearch/University of Copenhagen; Altropane; amyloid beta MRI contrast agents (Alzheimers), Mayo Clinic; amyloid binding PET ligands (Alzheimers disease), Aventis; AP-2011; ASP-1001; AZD-4694; AZPET; BAY-1006451; BAY-1006578; BAY-1163615; BAY-86-4367; BAY-86-7548; BAY-86-9596; BCI-632; BFPET; BR-14; BR-55; BY-963; CardioPET; Cavisomes; CB1 antagonists (brain imaging), Johns Hopkins; CEN-109; CGRP-A2 radioligand agent (migraine), Merck; CMC-001; CMUS-100; CNS-1261; CTP, Hafslund Nycomed; CTT-54; E-7210; EchoGen; Echovist; EM-2198; EM-3106B; EP-3533; F-18 exendin-4 derivative PET tracers (diabetes), Kyoto University/Arkray; F-18-CCR1; florbenazine (18F); florbetaben (18F); florbetapir (18F); florilglutamic acid (18F); Fluoratec; fluorescein derivative contrast agent (imaging, ocular disease), Philogen; fluorine-18-based PET imaging agents (neuropsychiatric disorders), Janssen; fluorine-18-labelled peptides (PET cancer imaging), Immunomedics; fluoropegylated indolylphenylacetylenes (Alzheimer's disease), Avid; flurpiridaz F 18; flutemetamol (18F); gadoversetamide; gallium-68 pasireotide tetraxetan; Gd contrast agents (liposomal nanoparticles), ImuThes Therapeutics; GE-226; GlyT1 PET radiotracers (schizophrenia), Merck & Co; GW-7845; humanized ATA antibodies (imaging, cancer), Enlyton; HumaSPECT; I-124-CLR1404; INO-4885; INP-04; intetumumab; iobitridol; iodixanol; iohexol; iomeprol; iopamidol; iopiperidol; iopromide; iosimenol; iotrolan (oral, X-ray imaging), Schering AG; Levovist; LMI-1195; MB-840; mGlu2 receptor PET ligand (psychiatric disease), Johnson & Johnson; mGluR5 PET tracers (neurodegenerative disease), Merck & Co; MN-3015; MRX-408; Myomap; N-1177-inh; N-1177-iv; N-1177-sq; nAChR PET agent, NIDA; NanoBarium; NanoLymph; NK3 antagonist PET ligand (psychiatric disease), AstraZeneca; NMDA radioligands, Kyushu University; NMK-36; nociceptin/orphanin FQ receptor PET ligands (neuropsychiatric disorders), Eli Lilly; NP-50511; NSI-1; NVLS/FMAU; NVLS/FX-18A; octafluoropropane; omacianine; Oralex; oxilan; PB-127; Pb-203 labeled [DOTA]-ReCCMSH targeted alpha particle-emitting radionuclides (cancer), AlphaMed/University of Missouri; peptide-based PET radiotracer (breast cancer), Stanford University Medical Center; perflexane-lipid microsphere; perflubutane (lipid microsphere-encapsulated, imaging), Daiichi Sankyo; perflubutane (polymer microsphere-encapsulated, heart disease), Acusphere; perflutren lipid microsphere; PET imaging agent (Alzheimer's disease), AC Immune; PET imaging agent (anti-5T4 tumor antigen Ab, ovarian cancer), ImaginAb; PET imaging agent (neurodegenerative diseases), Fujisawa; PET imaging agent (thrombosis), Astellas; PET imaging agents (cardiovascular disease), ImaginAb/GE Healthcare; PET radiotracer (prostate cancer), Johns Hopkins University School of Medicine; PET radiotracer (solid tumors), MD Anderson Cancer Center; phosphodiesterase 10 imaging agent (PET, neurological disorders), Institute for Neurodegenerative Disorders; PIMBA; Quantison; QW-7437; radiolabeled anti-CEACAM6 antibodies (imaging/cancer), NIH; radiolabeled anti-PSMA huJ591 minibodies (prostate cancer), ImaginAb; radiolabeled onartuzumab (imaging, cancer), Genentech; radiolabeled sigma-2 receptor ligands (solid tumor), Washington University in St Louis; radiolabeled WC-10 (neurological disease), Washington University; radiolabelled-A20FMDV2; RESP-3000; RG-7334; SH-U-563; SonoRx; SR-4554; STARBURST dendrimer-based MRI contrast agents (cardiovascular disease/ovary cancer), Dendritic Nanotechnology; sulphur hexafluoride microbubble ultrasound agent, Bracco; tau-binding PET tracer (Alzheimer disease), Siemens; T-cell co-receptor targeting PET imaging agent (antibody fragment, cancer/inflammation/transplantation), ImaginAb; technetium Tc 99m etarfolatide; technetium Tc 99m tilmanocept; TF-2 plus diagnostic/therapeutic (cancer), Immunomedics; TKS-040; TRC-105; triiodobenzene contrast agents, Nycomed; VasoPET; VMAT2 ligands (CNS disorder imaging), Molecular Neurolmaging/Institute for Neurodegenerative Disorders; yttrium (90Y) clivatuzumab tetraxetan; and any combinations thereof.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ). In certain other instances, the detectable response the detectable response is radioactivity (i.e., radiation), including alpha particles, beta particles, nucleons, electrons, positrons, neutrinos, and gamma rays emitted by a radioactive substance such as a radionuclide.

Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled aggregate. Positron emission tomography (PET) is another suitable technique for detecting radiation in a subject.

One of skill in the art will understand that the methods described herein for attaching ligands to the nanoparticles can be also be used for attaching imaging agents to the nanoparticles. In addition, an ordinarily skilled artisan will also be familiar with other methods for attaching imaging agents to nanoparticle and/or fabricating nanoparticles that comprise an imaging agent.

In some embodiments, the aggregate comprises at least one therapeutic agent and at least one imaging or contrast agent. This can be useful for simultaneous delivery of a therapeutic agen and an imaging or contrast agent for theranostic.

Without wishing to be bound by a theory, aggregation of nanoparticles into an aggregate reduces the rate of release and/or amount released of the compound(s) associated with the aggregate or prevents the compound(s) from coming in contact with the cells that would absorb or adsorb the compound(s). This can be due to the reduction in the surface area of aggregate relative to the total surface area of the individual nanoparticle. Accordingly, in some embodiments, the associated compound is released at a higher rate and/or amount from a disaggregated aggregate relative to release from to a non-disaggregated aggregate. For example, the rate of release from a disaggregated aggregate is at least 10%, at least 20%, at least 30%, at least 40%, at least 50%, at least 60%, at least 70%, at least 80%, at least 90%, at least 1-fold, at least 2-fold, at least 5-fold, at least 10-fold, at least 20-fold, at least 30-fold, at least 40-fold, at least 50-fold, or at least 100-fold or higher, relative to release from a non-disaggregated aggregate.

Because aggregation of nanoparticles into an aggregate reduces the rate of release and/or amount released of the compound(s) associated with the aggregate, the aggregates described herein can be used as slow release drug carriers to prolong circulating half-life of therapeutic agents. For example, aggregates that only undergo partial disaggregation under normal blood vessel shear stress will not release, or release very little, of the nanoparticles and a molecule associated therewith. This can increase circulation life of the nanoparticle and the associated therapeutic agent. Thus, aggregates that disaggregate partially, e.g., less than 20%, less than 15%, less than 10%, less than 5%, less than 4%, less than 3%, or less than 2% under normal blood vessel shear stress (e.g., less than 70 dyne/cm², less than 60 dyne/cm², less than 50 dyne/cm², less than 40 dyne/cm², less than 30 dyne/cm², less than 25 dyne/cm², less than 20 dyne/cm², or less than 15 dyne/cm²) can be used as slow release drug carriers to increase circulating half-life of therapeutic agents.

In some embodiments, the aggregates that disaggregate partially, e.g., less than 20%, less than 15%, less than 10%, less than 5%, less than 4%, less than 3%, or less than 2% in the shear stress range of from about 1 dyne/cm² to about 25 dyne/cm², from about 2 dyne/cm² to about 20 dyne/cm², or from about 5 dyne/cm² to about 15 dyne/cm² can be used as slow release drug carriers to increase the circulating half-life of therapeutic agents.

The compositions described herein can be used for controlled or extended release of therapeutic or imaging agents. For example, in vivo half-life of a therapeutic or imaging agent can be increased or decreased by encapsulating the agent in polymer systems with different delivery rates. The agent can be encapsulated in the aggregate. The agent can be conjugated to the nanoparticle, aggregate, RBC or microcapsule. The linkage between the agent and the nanoparticle, aggregate, RBC or microcapsule can be a cleavable or time-sensitive linkage. The particle size, shape and composition can also be varied to extend the half-life of therapeutic agents.

Additionally, this slow release can occur throughout the entire vasculature over time. This can be useful in long term targeting of endothelium under physiological shear stress conditions.

### Prodrug Delivery

The compositions and methods described herein can also be used for delivering a pro-drug and an agent for activating the prodrug. For example, the prodrug and the activating agent can be kept separate from each other in the aggregate. This can be accomplished, for example, by using nanoparticles which separately comprise (encapsulated or absorbed/adsorbed on the surface) the prodrug or the activating agent. In some embodiments, the prodrug can be encapsulated in the aggregate and the activating agent can be conjugated to the surface of the aggregate. In some other embodiments, the activating agent can be encapsulated in the aggregate and the prodrug can be conjugated (covalently or non-covaletly) to the surface of the aggregate. When the aggregate disaggregates the prodrug and the activating agent can come in contact (or interact) with each other releasing the drug. This can be used to safety match the delivery of both the prodrug and the activating agent to prodrug's desired site of action.

In one embodiment, the prodrug can be a polypeptide which becomes biologically active after cleavage or removal of a part thereof. The cleavage or removal of part of the polypeptide can be by enzymatic or chemical means. In one non-limiting example of this, the prodrug can be plasminogen and the activating agent can be a plasminogen activator. In some embodiments, the plasminogen activator can be urokinase, pro-urokinase, streptokinase, plasmin or, or tPA. The plasminogen can be encapsulated within the aggregate and the plasminogen activator can be conjugated to the outside surface of the aggregate.

### Ligands

A wide variety of entities can be coupled to the nanoparticles, microaggregates, red blood cells and microcapsules. Preferred moieties are ligands, which are coupled, preferably covalently, either directly or indirectly via an intervening tether. In preferred embodiments, a ligand alters the distribution, targeting or lifetime of the nanoparticle, red blood cell or microcapsule into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ligands providing enhanced aggregation are termed aggregating ligands herein.

Ligands providing enhanced affinity for a selected target are also termed targeting ligands herein. As used herein, the term “targeting ligand” refers to a molecule that binds to or interacts with a target molecule. Typically the nature of the interaction or binding is noncovalent, e.g., by hydrogen, electrostatic, or van der waals interactions, however, binding can also be covalent.

In some embodiments, the targeting ligand increases or enhances the efficiency or rate disaggregation of the aggregate at site of the target molecule or in the presence of the target molecule. For example, a targeting ligand can increase or enhance the efficiency or rate of disaggregation of the aggregate by at least 1%, at least 2%, at least 3%, at least 4%, at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, or more at site or presence of the target molecule relative to in the absence of the target molecule.

Without limitation, a ligand can be selected from the group consisting of peptides, polypeptides, proteins, enzymes, peptidomimetics, glycoproteins, antibodies and portions and fragments thereof, lectins, nucleosides, nucleotides, nucleic acids, monosaccharides, disaccharides, trisaccharides, oligosaccharides, polysaccharides, lipopolysaccharides, vitamins, steroids, hormones, cofactors, receptors, receptor ligands, and analogs and derivatives thereof.

In some embodiments of this and other aspects of the invention, the ligand is selected from the group consisting of CD47 or a fragment thereof, tPA, polylysine (PLL), intercellular adhesion molecules (ICAMS), cellular adhesion molecules (CAMS), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, polyphosphazine, polyethylenimine, cspermine, spermidine, polyamine, pseudopeptide-polyamine, peptidomimetic polyamine, dendrimer polyamine, arginine, amidine, protamine, thyrotropin, melanotropin, lectin, surfactant protein A, mucin, transferrin, bisphosphonate, polyglutamate, polyaspartate, an aptamer, asialofetuin, hyaluronan, procollagen, insulin, transferrin, albumin, acridines, cross-psoralen, mitomycin C, TPPC4, texaphyrin, Sapphyrin, polycyclic aromatic hydrocarbons (e.g., phenazine, dihydrophenazine), bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), RGD peptide, radiolabeled markers, haptens, naproxen, aspirin, dinitrophenyl, HRP, AP, lectins, vitamin A, vitamin E, vitamin K, vitamin B, folic acid, B12, riboflavin, biotin, pyridoxal, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, GalNAc, galactose, mannose, mannose-6P, clusters of sugars such as GalNAc cluster, mannose cluster, galactose cluster, an aptamer, integrin receptor ligands, chemokine receptor ligands, serotonin receptor ligands, PSMA, endothelin, GCPII, somatostatin, and any combinations thereof.

In some embodiments, the cell adhesion molecule (CAM) is an immunoglobulin, an integrin, a selectin or a cadherin.

In some embodiments, the ligand is monoclonal antibody or a fragment thereof. In some embodiments, the ligand is a polyclonal antibody of a fragment thereof.

In some embodiments of this and other aspects of the invention, the ligand is a peptide selected from the group consisting of, SEQ ID NO: 1 (CREKA), SEQ ID NO: 2 (CRKRLDRNK), SEQ ID NO: 3 (CHVLWSTRC), SEQ ID NO: 4 (ALEALAEALEALAEA), SEQ ID NO: 5 (KFFKFFKFFK (Bacterial cell wall permeating peptide)), SEQ ID NO: 6 (AALEALAEALEALAEALEALAEAAAAGGC (GALA)), SEQ ID NO: 7 (ALAEALAEALAEALAEALAEALAAAAGGC (EALA)), SEQ ID NO: 8 (GLFEAIEGFIENGWEGMIWDYG (INF-7)), SEQ ID NO: 9 (GLFGAIAGFIENGWEGMIDGWYG (Inf HA-2)), SEQ ID NO: 10 (GLF EAI EGFI ENGW EGMI DGWYGC GLF EAI EGFI ENGW EGMI DGWYGC (diINF-7)), SEQ ID NO: 11 (GLF EAI EGFI ENGW EGMI DGGC GLF EAI EGFI ENGW EGMI DGGC (diINF-3)), SEQ ID NO: 12 (GLFGALAEALAEALAEHLAEALAEALEALAAGGSC (GLF)), SEQ ID NO: 13 (GLFEAIEGFIENGWEGLAEALAEALEALAAGGSC (GALA-INF3)), SEQ ID NO: 14 (GLF EAI EGFI ENGW EGnI DG K GLF EAI EGFI ENGW EGnI DG (INF-5, n is norleucine)), SEQ ID NO: 15 (RQIKIWFQNRRMKWKK (penetratin)), SEQ ID NO: 16 (GRKKRRQRRRPPQC (Tat fragment 48-60)), SEQ ID NO: 17 (GALFLGWLGAAGSTMGAWSQPKKKRKV (signal sequence based peptide)), SEQ ID NO: 18 (LLIILRRRIRKQAHAHSK (PVEC)), SEQ ID NO: 19 (WTLNSAGYLLKINLKALAALAKKIL (transportan)), SEQ ID NO: 20 (KLALKLALKALKAALKLA (amphiphilic model peptide)), SEQ ID NO: 21 (RRRRRRRRR (Arg9)), SEQ ID NO: 22 (LLGDFFRKSKEKIGKEFKRIVQRIKDFLRNLVPRTES (LL-37)), SEQ ID NO: 23 (SWLSKTAKKLENSAKKRISEGIAIAIQGGPR (cecropin P1)), SEQ ID NO: 24 (ACYCRIPACIAGERRYGTCIYQGRLWAFCC (α-defensin)), SEQ ID NO: 25 (DHYNCVSSGGQCLYSACPIFTKIQGTCYRGKAKCCK ((3-defensin)), SEQ ID NO: 26 (RRRPRPPYLPRPRPPPFFPPRLPPRIPPGFPPRFPPRFPGKR-NH2 (PR-39)), SEQ ID NO: 27 ILPWKWPWWPWRR-NH2 (indolicidin)), SEQ ID NO: 28 (AAVALLPAVLLALLAP (RFGF)), SEQ ID NO: 29 (AALLPVLLAAP (RFGF analogue)), SEQ ID NO: 30 (RKCRIVVIRVCR (bactenecin)), cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, caerins, and any analogs and derivatives thereof.

In some embodiments, the targeting ligand can be selected from the group consisting of tPA fibrin (to target fibrin), von Willibrand factor (vWF) or a functional fragment thereof (to target platelets).

In some embodiments, the targeting ligand can be an antibody (monoclonal or polyclonal) and portions and fragments thereof.

In some embodiments of this and other aspects of the invention, the ligand is an aggregating ligand. Without wishing to be bound by a theory, an aggregating ligand can decrease the rate of disaggregation by at least 1%, at least 2%, at least 3%, at least 4%, at least 10%, at least 20%, at least 30%, at least 40%, at least 50%, at least 60%, at least 70%, at least 80%, or at least 90% or more, relative to a control.

In some embodiments, the ligand is a fluorescent reporter or a chemiluminescent molecule.

In some embodiments, of this and other aspects of the invention, a nanoparticle comprises both a targeting ligand and the target molecule. Without wishing to be bound by a theory, binding of the targeting ligand on one nanoparticle to a target molecule on a second nanoparticle enhances aggregation.

### Linking of Molecules

A molecule (e.g. a compound or a ligand) can be conjugated to a nanoparticle, red blood cell, or microcapsule using any of a variety of methods known to those of skill in the art. The molecule can be coupled or conjugated to the nanoparticle, red blood cell, or microcapsule covalently or non-covalently. The covalent linkage between the molecule and the nanoparticle, red blood cell, or microcapsule can be mediated by a linker. The non-covalent linkage between the molecule and the nanoparticle, red blood cell, or microcapsule can be based on ionic interactions, van der Waals interactions, dipole-dipole interactions, hydrogen bonds, electrostatic interactions, and/or shape recognition interactions.

Without limitations, conjugation can include either a stable or a labile (e.g. cleavable) bond or linker. Exemplary conjugations include, but are not limited to, covalent bond, amide bond, additions to carbon-carbon multiple bonds, azide alkyne Huisgen cycloaddition, Diels-Alder reaction, disulfide linkage, ester bond, Michael additions, silane bond, urethane, nucleophilic ring opening reactions: epoxides, non-aldol carbonyl chemistry, cycloaddition reactions: 1,3-dipolar cycloaddition, temperature sensitive, radiation (visible, IR, near-IR, UV, or x-ray) sensitive bond or linker, pH-sensitive bond or linker, noncovalent bonds (e.g., ionic charge complex formation, hydrogen bonding, pi-pi interactions, cyclodextrin/adamantly host guest interaction) and the like.

As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

Without limitations, any conjugation chemistry known in the art for conjugating two molecules or different parts of a molecule together can be used to for linking a molecule of interest (e.g. a drug) to a nanoparticle, red blood cell, or microcapsule. Exemplary linker and/or functional groups for conjugating a drug or ligand to a nanoparticle, red blood cell, or microcapsule include, but are not limited to, a polyethylene glycol (PEG, NH₂-PEG_{X}-COOH which can have a PEG spacer arm of various lengths X, where 1<X<100, e.g., PEG-2K, PEG-5K, PEG-10K, PEG-12K, PEG-15K, PEG-20K, PEG-40K, and the like), maleimide linker, PASylation, HESylation, Bis(sulfosuccinimidyl) suberate linker, DNA linker, Peptide linker, Silane linker, Polysaccharide linker, Hydrolyzable linker.

In some embodiments, the molecule can be covalently linked to the nanoparticle or microcapsule by a PEG linker. Without wishing to be bound by a theory, using a PEG based linker to attach the molecules to the nanoparticles provides a clinically relevant biocompatible strategy for fabricating the nanoparticles and the aggregates. An exemplary PEGylation approach for attaching an exemplary drug (tPA) to surface of nanoparticles is shown in FIG. 13. Carboxylic groups on surface of the nanoparticle can be activated using anyone of the methods and reagents available to the artisan. In some embodiments, the carboxylic groups can be activated using EDC/NHS chemistry. A heterobifunctional PEG (e.g., a heterobifunctional amino PEG acid) can be conjugated to the nanoparticles via a coupling between amines and activated carboxylic groups. The carboxylic groups on the PEG can be activated using anyone of the methods and reagents available to the artisan and conjugated with the molecule (e.g. drug) of interest via an amine group present inherently in the molecule or attached to the molecule.

The molecule (e.g., drug or ligand) can be conjugated with the nanoparticle, red blood cell, or microcapsule by an affinity binding pair. The term “affinity binding pair” or “binding pair” refers to first and second molecules that specifically bind to each other. One member of the binding pair is conjugated with the molecule while the second member is conjugated with the nanoparticle, red blood cell, or microcapsule. As used herein, the term “specific binding” refers to binding of the first member of the binding pair to the second member of the binding pair with greater affinity and specificity than to other molecules.

Exemplary binding pairs include any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin, hormone [e.g., thyroxine and cortisol-hormone binding protein, receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleoitde pairs capable of forming nucleic acid duplexes), and the like. The binding pair can also include a first molecule which is negatively charged and a second molecule which is positively charged.

One example of using binding pair conjugation is the biotin-avidin or biotin-streptavidin conjugation. In this approach, one of the molecule or nanoparticle, red blood cell, or microcapsule is biotinylated and the other is conjugated with avidin or streptavidin. Many commercial kits are also available for biotinylating molecules, such as proteins.

Another example of using binding pair conjugation is the biotin-sandwich method. See, e.g., example Davis et al., Proc. Natl. Acad. Sci. USA, 103: 8155-60 (2006). The two molecules to be conjugated together are biotinylated and then conjugated together using tetravalent streptavidin as a linker.

Still another example of using binding pair conjugation is double-stranded nucleic acid conjugation. In this approach, one of the molecule or nanoparticle, red blood cell, or microcapsule is conjugated with a first strand of the double-stranded nucleic acid and the other is conjugated with the second strand of the double-stranded nucleic acid. Nucleic acids can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges.

In some embodiments, the linker comprises at least one cleavable linking group, i.e., the linker is a cleavable linker. A cleavable linking group is one which is sufficiently stable under one set of conditions, but which is cleaved under a different set of conditions to release the two parts the linker is holding together. In a preferred embodiment, the cleavable linking group is cleaved at least 10 times or more, preferably at least 100 times faster under a first reference condition (which can, e.g., be selected to mimic or represent intracellular conditions, stenosis, or stenotic lesions) than under a second reference condition (which can, e.g., be selected to mimic or represent conditions found in the blood or serum).

Cleavable linking groups are susceptible to cleavage agents, e.g., hydrolysis, pH, redox potential, temperature, radiation, sonication, or the presence of degradative molecules (e.g., enzymes or chemical reagents), and the like. Generally, cleavage agents are more prevalent or found at higher levels or activities at a site of interest (e.g. stenosis or stenotic lesion) than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell, organ, or tissue to be targeted. In some embodiments, cleavable linking group is cleaved at least 1.25, 1.5, 1.75, 2, 3, 4, 5, 10, 25, 50, or 100 times faster under a first reference condition (or under in vitro conditions selected to intracellular conditions, stenosis, or stenotic lesions) than under a second reference condition (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to intracellular conditions, stenosis or stenotic lesions (or under in vitro conditions selected to mimic intracellular conditions, stenosis or stenotic lesions)

Exemplary cleavable linking groups include, but are not limited to, hydrolyzable linkers, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid clearable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease. In some embodiments, an acid cleavable linking group is cleavable in an acidic environment with a pH of about 6.5 or lower (e.g., about 6.5, 6.0, 5.5, 5.0, or lower), or by agents such as enzymes that can act as a general acid.

Activation agents can be used to activate the components to be conjugated together (e.g., surface of nanoparticle). Without limitations, any process and/or reagent known in the art for conjugation activation can be used. Exemplary surface activation method or reagents include, but are not limited to, 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3,3-tetramethyl uronium hexafluorophosphate Methanaminium (HATU), silinazation, surface activation through plasma treatment, and the like.

Again, without limitations, any art known reactive group can be used for coupling. For example, various surface reactive groups can be used for surface coupling including, but not limited to, alkyl halide, aldehyde, amino, bromo or iodoacetyl, carboxyl, hydroxyl, epoxy, ester, silane, thiol, and the like.

### Aggregate Fabrication

In one aspect, the invention provides a method for preparing an aggregate described herein, the method comprising: (i) fabricating a plurality of nanoparticles; (ii) aggregating said plurality of nanoparticle into micron sized particles. The fabricated nanoparticles may also be further subjected to centrifugation to decrease the concentration of single unbound nanoparticles in the aggregate. The fabricated nanoparticles may also be further subjected to centrifugation to decrease the concentration of single unbound nanoparticles in the aggregate.

After aggregation, particles of desired size can be selected by employing various techniques well known to a skilled artisan, such as size exclusion chromatography, use of track etched filters, sieving, filtering, and the like. In one non-limiting example, aggregated particles can be filtered using a filter with appropriate pore size. In another non-limiting example, aggregated particles can be subjected to density gradient centrifugation. Cheng et al. (Review of Scientific Instruments, 2010, 81: 026106), content of which is incorporated herein by reference, describes a method for high-precision microsphere sorting using velocity measurement. The method can be adapted to select aggregates of desired size.

Accordingly, in some embodiments, the method further comprises the step of selecting aggregated particles ≧1 μm, ≧2 μm, ≧3 μm, ≧4 μm, ≧5 μm, ≧6 μm, ≧7 μm, ≧8 μm, ≧8 μm, or ≧10 μm in size.

In some embodiments, the method further comprises the step of selecting aggregated particles ≦20 μm, ≦15 μm, ≦10 μm, or ≦5 μm in size.

In some embodiments, the method further comprises selecting aggregated particles of a certain size range, e.g., from 1 μm to 50 μm, from 1 μm to 25 μm, from 1 μm to 20 μm, from 1 μm to 10 μm, or from 0.5 μm to 5 μm. This can be accomplished by first selecting particles of size less than the upper size limit and then from those particles selecting particles of size greater than the lower size limit or vice-versa.

Various methods can be employed to fabricate nanoparticles of suitable size for aggregation. These methods include vaporization methods (e.g., free jet expansion, laser vaporization, spark erosion, electro explosion and chemical vapor deposition), physical methods involving mechanical attrition (e.g., the pearl milling technology developed by Elan Nanosystems of Dublin, Ireland), and interfacial deposition following solvent displacement.

In some embodiments, the MICROSIEVE™ emulsification technology by Nanomi can be used for producing narrow size distribution particles. The MICROSIEVE™ emulsification technology is described on the web at www.nanomi.com/membrane-emulsification-technology.html.

The solvent displacement method is relatively simple to implement on a laboratory or industrial scale and can produce nanoparticles able to pass through a 0.22 μm filter. The size of nanoparticles produced by this method is sensitive to the concentration of polymer in the organic solvent, to the rate of mixing, and to the surfactant employed in the process. Although use of the solvent displacement method with the surfactant sodium dodecyl sulfate (SDS) has yielded small nanoparticles (<100 nm), SDS is not ideal for a pharmaceutical formulation. However, similar natural surfactants (e.g., cholic acid or taurocholic acid salts) can be substituted for SDS to obtain similarly sized nanoparticles. Taurocholic acid, the conjugate formed from cholic acid and taurine, is a fully metabolizable sulfonic acid with very similar amphipathic solution chemistry to SDS. An analog of taurocholic acid, tauroursodeoxycholic acid (TUDCA), is not toxic and is actually known to have neuroprotective and anti-apoptotic properties. TUDCA is a naturally occurring bile acid and is a conjugate of taurine and ursodeoxycholic acid (UDCA). UDCA is an approved drug (ACTIGALL®, Watson Pharmaceuticals) for the treatment of gallbladder stone dissolution. Other naturally occurring anionic surfactants (e.g., galactocerebroside sulfate), neutral surfactants (e.g., lactosylceramide) or zwitterionic surfactants (e.g., sphingomyelin, phosphatidyl choline, palmitoyl carnitine) can be used in place of SDS or other surfactants that have been commonly employed in nanoparticle formulation studies. Other excipients that are generally recognized as safe, such as those used to solubilize the basic form of gacyclidine, can also be used to prepare nanoparticles. Such excipients include a polyoxyethylene fatty acid ester (e.g., polysorbate 80 (e.g., TWEEN 80®)), a polyglycol mono or diester of 12-hydroxy steric acid (e.g., SOLUTOL® HS 15), and CAPTISOL®. Poloxamers such as (but not limited to) poloxamer 407 can also be used.

A sampling of various surfactants can be used in order to determine the optimal surfactants for small (e.g., <200 nm), non-toxic drug-containing nanoparticles. Surfactant concentrations also affect the formation of the nanoparticles, their density and their size. A surfactant concentration can be optimized for each polymer composition, desired drug concentration, and intended use.

Of the various organic solvents previously employed in nanoparticle formulation, acetone is attractive because of its prior use in preparing filterable nanoparticles, its low toxicity, and its ease of handling. Various polymers composed of L- and D,L-lactic acid (PLA) or mixtures of lactic acid and glycolic acid (poly(lactide-co-glycolide)) (PLGA) are soluble in acetone, with the exception of 100% L-PLA and 100% glycolic acid (PGA). Polymers composed of 100% L-PLA will dissolve in methylene chloride and polymers composed of either 100% L-PLA or 100% PGA will dissolve in hexafluoroisopropanol (HFIP).

Rapid mixing can be employed when preparing nanoparticles using the solvent displacement method. In some such embodiments, a stirring rate of 500 rpm or greater is typically employed. Slower solvent exchange rates during mixing result in larger particles. Fluctuating pressure gradients are used to produce high Reynolds numbers and efficient mixing in fully developed turbulence. Use of high gravity reactive mixing has produced small nanoparticles (10 nm) by achieving centrifugal particle acceleration similar to that achieved by turbulent mixing at high Reynolds numbers.

Sonication is one method that can provide turbulent mixing. Sonication is the method most commonly employed with the double emulsion nanoparticle fabrication method, but is less suited to the solvent displacement method. Sonication can be performed by mixing two liquid streams (e.g. one stream having dissolved particle polymeric material and the other stream having a drug and/or combination of drugs that will cause the particles to come out of solution and solidify) passing through a tube with an inline ultrasonic vibrating plate at the point of stream intersection. Formation of very small liquid droplets by vibrational atomization has also been employed in the fabrication of nanoparticles. For example, the DMP-2800 MEMS-based piezoelectric micropump (inkjet) system produced by the Spectra Printing Division (Lebanon, N.H.) of Dimatix, Inc. (Santa Clara, Calif.) forms a 10-50 pL (1-5×10⁻¹¹ liter) sized liquid droplet at 100,000 pL/s. Micropumps (inkjet systems) offer uniform mixing and the ability to reliably translate the process from lab to production scale, but production of nanoparticles smaller than 200 nm will still rely on mixing dynamics (i.e., the solidification timing of the precipitated solid or liquid intermediates produced on mixing) when piezoelectric micropumps are used to produce small, polymer-laden droplets. Temperature, surfactant and solvent composition are important variables in using this approach, as they modify the solidification dynamics and the density of the produced nanoparticle.

The nanoparticles can be induced to form aggregates by a wide variety of methods available and well known to the skilled artisan. Many hydrophobic nanoparticles, such PLGA based nanoparticles, can self-aggregate in aqueous solution. See for example, C. E. Astete and C. M. Sabliov, J. Biomater. Sci, Polymer Ed. 17:247 (2006). Accordingly, a concentrated solution comprising the nanoparticles can be stored at room temperature or lower temperature for a period of time. In some embodiments, the storage temperature is 4° C. or lower. Without limitation, the storage period can last from minutes to days or weeks. For example, the storage period is 1-day, 2-days, 3-days, 4-days, 5-days, 6-days, 1-week, 2-week or more.

Alternatively, a concentrated solution of nanoparticles can be spray dried to form aggregates. See for example, Sung, et al., Pharm. Res. 26:1847 (2009) and Tsapis, et al., Proc. Natl. Acad. Sci. USA, 99:12001 (2002).

The concentrated solution can comprise 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml or more of the nanoparticles.

Other methods of forming aggregates include, but are not limited to, the w/o/w emulsion method and the simple solvent displacement method.

In one non-limiting example, the nanoparticles are fabricated from PLGA polymers. The PLGA polymer can be conjugated with PEG and/or a ligand. Accordingly, in some embodiments of this and other aspects of the invention, the nanoparticles are fabricated from PEG-PLGA polymers to which the peptide CREKA (SEQ ID NO: 1), CRKRLDRNK (SEQ ID NO: 2), or CHVLWSTRC (SEQ ID NO: 3) is linked. The CREKA (SEQ ID NO: 1) peptide is known to home in to a wide variety of tumors. Without wishing to be bound by a theory, the CREKA (SEQ ID NO: 1) peptide recognizes clotted blood, which is present in the lining of tumor vessels but not in vessels of normal tissues. Additionally, CREKA (SEQ ID NO: 1) peptide is used to target fibrin located on the luminal surface of atherosclerotic plaque.

The CRKRLDRNK (SEQ ID NO: 2) peptide is a known peptide targeting inflamed endothelium.

The CHVLWSTRC (SEQ ID NO: 3) peptide is a known peptide, which targets islet endothelial cells.

### Drug or Contrast/Imaging Agent Delivery

In another aspect, the invention provides a method of delivering or controlling the release of a drug or contrast/imaging molecule at a desired site in a subject. The method comprising administering to a subject in need thereof an aggregate described herein. In some embodiments, the method further comprising applying or administering an external stimulus, e.g. ultrasound, magnetic, radiation (e.g., visible, UV, IR, near-IR), temperature, pressure, and the like, to the subject. Without wishing to be bound by a theory, this external stimulus can disaggregate the aggregate thereby releasing the therapeutic agent or imaging agent comprised in the aggregate.

The methods disclosed herein differ from the art known drug delivery methods employing an external stimulus for drug delivery. The art known methods are based on rupture of micro-bubbles or liposomes. The drug is encapsulated in the cavity of micro-bubbles/liposomes and the external stimulus ruptures the micro-bubble or the liposome. For example, high intensity ultrasound is used to break up the micro-bubble/liposome and requires complex equipment. Use of high intensity ultrasound can cause local tissue damage and can be too harmful for non-cancer or non-acute treatments. In contrast, the method disclosed herein is based on disaggregation of aggregates and dispersing nanoparticles with a stimulus. For example, ultrasound can be used to disaggregate the aggregate to disperse the nanoparticles. Without wishing to be bound by a theory, the method and aggregates disclosed herein allows use of lower intensity of ultrasound for delivering a drug to a desired site in a subject. For example, ultrasound intensity can be equal to or less than about 150 W/cm⁻², 125 W/cm⁻², 100 W/cm⁻², 75 W/cm⁻², 50 W/cm⁻², 25 W/cm⁻², 20 W/cm⁻², 15 W/cm⁻², 10 W/cm⁻², 7.5 W/cm⁻², 5 W/cm⁻², or 2.5 W/cm⁻². In some embodiments, the ultrasound intensity can be between 0.1 W/cm⁻² and 20 W/cm⁻²; between 0.5 W/cm⁻² and 15 W/cm⁻²; or between 1 W/cm⁻² and 10 W/cm⁻².

Further, the aggregates and methods disclosed herein provide controlled release of the molecule (e.g. drug) from the nanoparticle over time as opposed to the burst release from current proposed carriers. Moreover, the methods and aggregates can provide drug targeting and delivery at a desire site by combining targeting moieties on the nanoparticles or aggregates.

While the following section discusses applications of the compositions and methods described herein to specific diseases, it is to be understood that the compositions and methods described herein can be used for delivery of therapeutic agents or imagining or contrast agents in a subject in need thereof.

### Treatment of Stenosis

In another aspect, the invention provides a method for treating stenosis and/or a stenotic lesion in a subject, the method comprising administering to a subject in need thereof an aggregate described herein.

As used herein, the term “stenosis” refers to narrowing or stricture of a hollow passage (e.g., a duct or canal) in the body. The term “vascular stenosis” refers to occlusion or narrowing of a canal or lumen of the circulatory system. Vascular stenosis often results from fatty deposit (as in the case of atherosclerosis), excessive migration and proliferation of vascular smooth muscle cells and endothelial cells, acute narrowing due to clot formation, or as a result of vascular malformation. As used herein, the term “vascular stenosis” includes occlusive lesions. Arteries are particularly susceptible to stenosis. The term “stenosis” as used herein specifically includes initial stenosis and restenosis. Typical examples of blockages within a canal or lumen include in situ or embolized atheromatous material or plaques, aggregations of blood components, such as platelets, fibrin and/or other cellular components, in clots resulting from disease or injury or at the site of wound healing. Clot-forming conditions include thrombosis, embolisms and in an extreme case, abnormal coagulation states. Other vascular blockages include blockages resulting from an infection by a microorganism or macroorganism within the circulatory system, such as fungal or heartworm infections. Sickle cell disease also can result in vessel obstruction as a result of RBC sickling and stacking into structures that are larger than the lumen of the microvessel. Thus, during sickle cell crisis, RBC change shape/stiffness and can occlude blood vessel. This phenomenon is also present during crisis stages of malaria. As such, as used herein, the term “vascular stenosis” includes arterial occlusive disease.

The term “restenosis” refers to recurrence of stenosis after treatment of initial stenosis with apparent success. For example, “restenosis” in the context of vascular stenosis, refers to the reoccurrence of vascular stenosis after it has been treated with apparent success, e.g. by removal of fatty deposit by balloon angioplasty. One of the contributing factors in restenosis is intimal hyperplasia. The term “intimal hyperplasia”, used interchangeably with “neointimal hyperplasia” and “neointimal formation”, refers to thickening of the inner most layer of blood vessels, intimal, as a consequence of excessive proliferation and migration of vascular smooth muscle cells and endothelial cells. The various changes taking place during restenosis are often collectively referred to as “vascular wall remodeling.” Without limitations, compositions and methods described herein can be used treat stent restenosis.

The terms “balloon angioplasty” and “percutaneous transluminal coronary angioplasty” (PTCA) are often used interchangeably, and refer to a non-surgical catheter-based treatment for removal of plaque from the coronary artery. Stenosis or restenosis often lead to hypertension as a result of increased resistance to blood flow.

The term “hypertension” refers to abnormally high blood pressure, i.e. beyond the upper value of the normal range.

Some exemplary causes of stenosis and/or stenotic lesion include, but are not limited to, trauma or injury, atherosclerosis, cerebral vasospasms, birth defects, diabetes, iatrogenic, infection, inflammation, ischemia, neoplasm, vasospasm, coronary vasospasm, Raynaud's phenomenon, stroke, blood clotting, Moyamoya disease, Takayasu's disease, polyarteritis nodosa, disseminated lupus erythematous, rheumatoid arthritis, tumors of the spine, Paget's disease of bone, fluorosis, extracorporeal devices (e.g., hemodialysis, blood pumps, etc.), thrombotic and/or embolic disorders, sickle cell anemia, and any combinations thereof.

As used herein, the term “thrombotic and/or embolic disorders” means acute or chronic pathological states or conditions resulting from occlusion or partial occlusion of a blood vessel due to thrombus or embolus. Similarly, the term “thrombotic or embolic occlusion” means occlusion or partial occlusion of a blood vessel due to thrombus or embolus. Examples of thrombotic and embolic disorders include, but are not limited to cerebral thrombotic and embolic disorders such as cerebral infarct (stroke), transient ischemic attack and vascular dementia; thrombotic and embolic disorders of the heart such as myocardial infarct, acute coronary syndrome, unstable angina and ischemic sudden death; renal infarcts, peripheral circulatory disorders and deep vein thrombosis.

In some embodiments of this and other aspects of the invention, stenosis or stenotic lesion is selected from the group consisting of arterial occlusive disease; a blood clot; intimal hyperplasia; stent restenosis; intermittent claudication (peripheral artery stenosis); angina or myocardial infraction (coronary artery stenosis); carotid artery stenosis (leads to strokes and transient ischaemic episodes); aortic stenosis; buttonhole stenosis; calcific nodular stenosis; coronary ostial stenosis; double aortic stenosis; fish-mouth mitral stenosis; idiopathic hypertrophic subaortic stenosis; infundibular stenosis; mitral stenosis; muscular subaortic stenosis; subaortic stenosis; pulmonary arterial stenosis; heart valve disease (valvular stenosis); subvalvar stenosis; supravalvar stenosis; tricuspid stenosis; renal artery stenosis; aneurysm; mesenteric artery thrombosis; venous stenosis; venous thrombosis; a lesion; disease or disorder of a fluid containing channel; and any combinations thereof.

### Treatment of Internal Hemorrhage

As used herein, the term “internal hemorrhage” refers to bleeding that is occurring inside the body. Such bleeding can be a serious depending on wherein it occurs (e.g., brain, stomach, lungs), and can potentially cause death and cardiac arrest if proper medical treatment is not quickly received. Accordingly, in one aspect, the invention provides a method for treating internal hemorrhage or a hemorrhagic disorder in a subject, the method comprising administering to a subject in need thereof an aggregate described herein. Depending on the nature of hemorrhage, the shear stress can be high at or near the bleed site.

Internal hemorrhage can result from a trauma, blood vessel rupture from high blood pressure, infection (e.g., Ebola, Marburg), cancer, scurvy, hepatoma, autoimmune thrombocytopenia, ectopic pregnancy, malignant hypothermia, ovarian cysts, liver cancer, vitamin K deficiency, hemophilia, or adverse effect of a medication.

As used herein the term “hemorrhagic disorder” means acute or chronic pathological state or condition resulting from bleeding from damaged blood vessel. Examples of hemorrhagic disorders include, but are not limited to, cerebral hemorrhages such as intracerebral hemorrhage (ICH), subarachnoid hemorrhage (SAH) and hemorrhagic stroke.

The aggregates described herein can also be used in extracorporeal devices, such as hemodialysis devices (possibly also artificial blood vessel/valves etc.)—these can cause elevated shear stress that induce shear activation of platelets etc. Aggregates described herein can be added to the extracorporeal device to release anti-platelets drug when elevated shear stress exists in these extracorporeal devices.

The aggregates can also be used for detection of abnormal flow in the body/extracorporeal devices. For example by measuring the release rate and/or amount of a label molecule.

Additionally, the aggregates can also be used in combination with embolization treatments. Embolization refers to the introduction of various substances into the circulation to occlude vessels, either to arrest or prevent hemorrhaging; to devitalize a structure, tumor, or organ by occluding its blood supply; or to reduce blood flow to an arteriovenous malformation. Thus, embolization includes selective occlusion of blood vessels by purposely introducing emboli in the blood vessels. Embolization is used to treat a wide variety of conditions affecting different organs of the human body, including arterivenous malformations, cerebral aneurysm, gastrointestinal bleeding, epistaxis, primary post-partum hemorrhage, surgical hemorrhage, slow or stop blood supply thus reducing the size of a tumor, liver lesions, kidney lesions and uterine fibroids.

In some embodiments, the aggregates can be used in combination with embolization treatment to clear an already occluded vessel. For example, an occluded vessel can be further embolized near or at site of the occlusion and the aggregate comprising an occlusion clearing molecule delivered to the site. Without wishing to be bound by a theory, this can be useful in cases where the occlusion in the vessel is insufficient to disaggregate the aggregate by itself.

### Pharmaceutical Compositions

For administration to a subject, the aggregates can be provided in pharmaceutically acceptable compositions. These pharmaceutically acceptable compositions comprise an aggregate, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, excipient can include Leucine, Mannitol, Sodium glycocholate, Trehalose, Sucrose, Dextran, PVA (polyvinyl alcohol), Cellulose, Cellulosic ethers, HPC, Polyox, Saccharides, Gelatin, and the like.

In some embodiments, a therapeutic agent or an imaging agent can be used as the excipient.

In some embodiments of this and other aspects of the invention, a therapeutically effective amount of therapeutic agent is administered to the subject. The phrase “therapeutically-effective amount” as used herein means that amount of a therapeutic agent which is effective for producing some desired therapeutic effect in at least a sub-population of cells in an animal at a reasonable benefit/risk ratio applicable to any medical treatment. For example, an amount of a therapeutic agent administered to a subject that is sufficient to produce a statistically significant, measurable modulation of stenosis.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. Routes of administration suitable for the methods of the invention include both local and systemic administration. Generally, local administration results in more of the therapeutic agent being delivered to a specific location as compared to the entire body of the subject, whereas, systemic administration results in delivery of the therapeutic agent to essentially the entire body of the subject.

Administration to a subject can be by any appropriate route known in the art including, but not limited to, oral or parenteral routes, including intravenous, intramuscular, subcutaneous, transdermal, rectal, and topical (including buccal and sublingual) administration.

Exemplary modes of administration include, but are not limited to, injection, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments of the aspects described herein, administration is by intravenous infusion or injection.

The microaggregate, RBC, or microcapsule described herein can be administered to a subject in conjunction with other art known therapies for removal of blood vessel obstructions. For example, the compositions and methods described herein can be used in combination with an endovascular (e.g. catheter-based) procedure. In some embodiments, the composition, e.g., microaggregate, RBC, or microcapsule, can be administered using a catheter. Without limitations, a catheter can be used to create a small opening in vascular obstruction (e.g. a clot). This can initiate flow and delivery of the appropriate therapeutic agent by the microaggregate, RBC, or microcapsule can remove or reduce the amount of left over obstruction (e.g. clot). The microaggregates, RBCs, or microcapsules can also be used to clear blood vessels of partial obstruction or restenosis resulting from catheter-based clot removal. In some embodiments, the aggregates and methods described herein can be used in combination with a second therapy comprising placement of a wire through an occlusion.

In some embodiments, the aggregates and methods described herein can be used in combination with mechanical thrombectomy. For example, mechanical thrombectomy be used to remove the obstruction while co-administering the aggregate described herein. For example, the aggregates and methods disclosed herein can be used with a retrievable stent (stentriever) or self-expanding stent. When used in combination with mechanical thrombectomy, the aggregate can be administered locally at the site of obstruction or stenosis.

In some embodiments, the aggregates and methods described herein can be used in combination with embolization treatments.

In some embodiments, the microaggregate, RBC, or microcapsule can be co-administered with an art known obstruction clearing agent for clearing or removing blood vessel obstructions. For example, an art known obstruction clearing agent for clearing or removing blood vessel obstructions can be administered to a subject. Without wishing to be bound by a theory, such an agent can induce or initiate some flow. Administering of the microaggregate, RBC, or microcapsule described herein can then be used to clear up the remaining obstructions. The microaggregate, RBC, or microcapsule and the obstruction clearing agent can be co-administered in the same composition of different compositions. When microaggregate, RBC, or microcapsule and the obstruction clearing agent are to be administered in different compositions, they can administered at the same time, e.g., within 30 seconds, one minute, two minutes, or three minutes of each other. Alternatively, the obstruction clearing agent can be administered first. The microaggregate, RBC, or microcapsule can then be administered within 1, 2, 3, 4, 5, 6, 7, 8, 9, 10, 15, 20, 25, 30, 35, 40, 45, 50, 55, 60, 90, 120, 150, 180, 210, 240, 270, 300, 330, or 360 minutes of administering of the obstruction clearing agent. It is not necessary to administer the recommended dosage of the obstruction clearing agent for this. An amount of obstruction clearing agent sufficient to induce or initiate flow at the obstruction can be used. In one example, a small amount of free tPA can be co-administered to the subject to initiate flow at the obstruction.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation. In addition, the methods and compositions described herein can be used to treat domesticated animals and/or pets.

A subject can be one who has been previously diagnosed with or identified as suffering from or having a disease or disorder characterized with stenosis or stenotic lesion, or a hemodynamic disorder or condition.

A subject can be one who is currently being treated for stenosis, stenotic lesion, a disease or disorder characterized with stenosis or stenotic lesion, or a hemodynamic disorder or condition.

A subject can be one who has been previously diagnosed with or identified as suffering from or having internal bleeding.

A subject can be one who is being treated for internal bleeding.

In some embodiments of the aspects described herein, the method further comprising diagnosing a subject for stenosis, stenotic lesion, internal bleeding, or a hemodynamic disorder or condition before onset of the treatment according to methods of the invention.

In some embodiments of the aspects described herein, the method further comprising selecting a subject with stenosis, stenotic lesion, internal bleeding, or a hemodynamic disorder or condition before onset of the treatment according to methods of the invention.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage can be determined by a physician and adjusted, as necessary, to suit observed effects of the treatment. Generally, the compositions are administered so that therapeutic agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compositions are administered at a dosage so that therapeutic agent or a metabolite thereof has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the therapeutic agent. The desired dose can be administered every day or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### Non-Medical Uses

The aggregates described herein are also useful in industrial applications. For example, the aggregates can be used for clearing a clogged pipe and/or to repair leaks in a pipe. Without limitation, a pipe can be of any diameter and any substance can be flowing through the pipe, e.g., chemicals, water, oil, gas, etc. As used herein, the term “pipe” is intended to include any type of apparatus through which a fluid can flow. Examples include chemical feed systems, municipal services, and supply pipelines, such as water, gas, and oil. As used herein the term “fluid” refers to a material that can flow. Accordingly, the term “fluid” includes liquid, gaseous, and semi-solid materials.

Without wishing to be bound by a theory, the shear stress in the clogged area is higher than in the unclogged area. Thus, the aggregate will disaggregate near or at the clogged area releasing agents that can clear the clog. Alternatively, or in addition, the aggregate can be placed at or near the desired site and an external stimulus applied to disaggregate the aggregate. Accordingly, in some embodiments of this and other aspects of the invention, the aggregate includes an agent that can unclog a pipe. Agents for unclogging a pipe can include, but are not limited to, agents capable of producing an exothermic reaction, producing an oxidation reaction, producing an enzymatic reaction, and any combinations thereof.

Agents that can produce an exothermic reaction can include a combination of a base and metal. The base and the metal can be formulated in separate aggregates, and an exothermic reaction takes place upon release of the base and metal at the clog, which can clear the clog. In some embodiments, base is sodium hydroxide. In some embodiments, the metal is aluminum.

Agents that can produce an oxidation reaction can include peroxygens, such as sodium percarbonate, sodium persulfate, and sodium perborate; and halogen-containing oxidizing compounds, such as calcium hypochlorite, alkali earth metal hypochlorites, alkaline earth metal hypochlorites, sodium dichloro-striazinetrione, chlorinated isocyanurates, 1,3-dibromo and 1,3-dichloro-5-isobutylhydantoin. In some embodiments, the oxidation agent includes a combination of a peroxygen and an organic substance, e.g., a carbohydrate.

Agents that can produce an enzymatic reaction can include bacterium. The bacterium can be a lignin-degrading bacterium. In some embodiments of this and other aspects of the invention, the bacterium produces at least one of a lipase, an amylase, a cellulase, or a protease.

In addition to the anti-clogging agent, the aggregate can include a compound selected from the group consisting of surfactants, slip agents, foam suppressants, anti-caking agents, binding agents, abrasive agents, corrosion inhibitors, defoamers, and any combinations thereof.

As discussed above, flow thorough a leak can also lead to high shear stress. Depending on the nature of the leak, shear stress can be high at or near the leak. Accordingly, in some embodiments of this and other aspects of the invention, the aggregate includes a sealing material. Exemplary sealing materials include, but are not limited to, alginates, particulates, mineral oils, silicone rubber, thermoplastic or thermosetting resins (vinyl acetate resins, or atactic polypropylene), rubber latexes, non-silicone type rubber (natural rubber (MR), isoprene rubber (IR), butadiene rubber (BR), poly(l, 2-butadiene) (1,2-BR), styrene-butadiene rubber (SBR), chloroprene rubber (CR), nitrile rubber (NBR), butyl rubber (TfR), ethylene-propylene rubber (EPM, EPDM), chlorosulfonated polyethylene (CSM) and acryl rubber (ACM, ANM).

### Kits

In another aspect, the invention provides a kit comprising an aggregate, a formulation comprising an aggregate, components for making an aggregate or a formulation comprising an aggregate described herein.

In addition to the above mentioned components, the kit can include informational material. The informational material can be descriptive, instructional, marketing or other material that relates to the methods described herein and/or the use of the aggregates for the methods described herein. For example, the informational material describes methods for administering the aggregate to a subject. The kit can also include a delivery device.

In one embodiment, the informational material can include instructions to administer the formulation in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments the individual components of the formulation can be provided in one container. Alternatively, it can be desirable to provide the components of the formulation separately in two or more containers, e.g., one container for an oligonucleotide preparation, and at least another for a carrier compound. The different components can be combined, e.g., according to instructions provided with the kit. The components can be combined according to a method described herein, e.g., to prepare and administer a pharmaceutical composition.

In addition to the formulation, the composition of the kit can include other ingredients, such as a solvent or buffer, a stabilizer or a preservative, and/or a second agent for treating a condition or disorder described herein. Alternatively, the other ingredients can be included in the kit, but in different compositions or containers than the formulation. In such embodiments, the kit can include instructions for admixing the formulation and the other ingredients, or for using the oligonucleotide together with the other ingredients.

The formulation can be provided in any form, e.g., liquid, dried or lyophilized form. It is preferred that the formulation be substantially pure and/or sterile. When the formulation is provided in a liquid solution, the liquid solution preferably is an aqueous solution, with a sterile aqueous solution being preferred. When the formulation is provided as a dried form, reconstitution generally is by the addition of a suitable solvent. The solvent, e.g., sterile water or buffer, can optionally be provided in the kit.

In some embodiments, the kit contains separate containers, dividers or compartments for the formulation and informational material. For example, the formulation can be contained in a bottle, vial, or syringe, and the informational material can be contained in a plastic sleeve or packet. In other embodiments, the separate elements of the kit are contained within a single, undivided container. For example, the formulation is contained in a bottle, vial or syringe that has attached thereto the informational material in the form of a label.

In some embodiments, the kit includes a plurality, e.g., a pack, of individual containers, each containing one or more unit dosage forms of the formulation. For example, the kit includes a plurality of syringes, ampules, foil packets, or blister packs, each containing a single unit dose of the formulation. The containers of the kits can be air tight and/or waterproof.

Compositions and methods for shear-stress controlled drug delivery are also described in International patent application no. PCT/US2011/049691, filed Aug. 30, 2011, content of which is incorporated herein by reference.

Some exemplary embodiments of the invention can be described by one or more of the following paragraphs:
1. An aggregate comprising a plurality of nanoparticles, wherein the aggregate disaggregates under a predetermined stimulus selected from the group consisting of ultrasound, mechanical strain, vibration, magnetic field, radiation, temperature, ionic strength, pH, pressure, turbulence, change in flow, flow rate, or chemical or enzymatic activation.
2. The aggregate of paragraph 1, wherein the aggregate further comprises a molecule selected from the group consisting of small or large organic or inorganic molecules; carbon-based materials (e.g., nanotubes, fullerenes, buckeyballs, and the like); metals; metal oxides; complexes comprising metals; inorganic nanoparticles; metal nanoparticles; monosaccharides; disaccharides; trisaccharides; oligosaccharides; polysaccharides; glycosaminoglycans; biological macromolecules; enzymes; amino acids; peptides; proteins; peptide analogs and derivatives thereof; peptidomimetics; antibodies and portions or fragments thereof; lipids; carbohydrates; nucleic acids; polynucleotides; oligonucleotides; genes; genes including control and termination regions; self-replicating systems such as viral or plasmid DNA; RNA; modified RNA; single-stranded and double-stranded siRNAs and other RNA interference reagents; short-hairpin RNAs (shRNA); hairpin DNAs; self-assemblying DNAs or RNAs; antisense oligonucleotides; ribozymes; microRNAs; microRNA mimics; aptamers; antimirs; antagomirs; triplex-forming oligonucleotides; RNA activators; immuno-stimulatory oligonucleotides; decoy oligonucleotides; nucleic acid analogs and derivatives; an extract made from biological materials such as bacteria, plants, fungi, or animal cells or tissues; naturally occurring or synthetic compositions; or any combinations thereof.
3. The aggregate of paragraph 2, wherein the antibody is a monoclonal antibody or fragment thereof or a polyclonal antibody or fragment thereof.
4. The aggregate of paragraph 2 or 3, wherein the molecule is non-covalently linked to the aggregate or the nanoparticle constituent of the aggregate.
5. The aggregate of any of paragraphs 1-4, wherein the molecule is non-covalently linked to the aggregate or the nanoparticle constituent of the aggregate by ionic interactions, van der Waals interactions, dipole-dipole interactions, hydrogen bonding, electrostatic interactions, shape recognition interactions, ionic charge complex formation, pi-pi interactions, and host guest interaction (e.g., cyclodextrin/adamantine).
6. The aggregate of any of paragraphs 1-5, wherein the molecule is absorbed/adsorbed on the surface of the aggregate or the nanoparticle constituent of the aggregate.
7. The aggregate of any of paragraphs 1-6, wherein the molecule is encapsulated in the aggregate or the nanoparticle constituent of the aggregate.
8. The aggregate of any of paragraphs 1-7, wherein the molecule is covalently linked to the aggregate or the nanoparticle constituent of the aggregate.
9. The aggregate of any of paragraphs 1-8, wherein the molecule is covalently linked to the aggregate or the nanoparticle constituent of the aggregate by a linker or functional group selected from the group consisting of a PEG linker, maleimide linker, PASylation, HESylation, bis(sulfosuccinimidyl) suberate linker, nucleic acid linker, peptide linker, silane linker, polysaccharide linker, bond, amide bond, additions to carbon-carbon multiple bonds, azide alkyne Huisgen cycloaddition, Diels-Alder reaction, disulfide linkage, ester bond, Michael additions, silane bond, urethane, nucleophilic ring opening reactions: epoxides, non-aldol carbonyl chemistry, cycloaddition reactions: 1,3-dipolar cycloaddition, tosylation, temperature sensitive, radiation (IR, near-IR, UV) sensitive bond or linker, pH-sensitive bond or linker, and a hydrolysable) linker.
10. The aggregate of any of paragraphs 1-9, wherein surface of the aggregate or the nanoparticle constituent of the aggregate is activated for linking with the molecule by a reagent selected from the group consisting of 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3,3-tetramethyl uronium hexafluorophosphate Methanaminium (HATU), tosylation, silinazation, and surface activation through plasma treatment.
11. The aggregate of any of paragraphs 1-10, wherein the aggregate or the nanoparticle constituent of the aggregate comprises a surface reactive group for linking with the molecule, wherein the surface reactive group is selected from the group consisting of alkyl halide, aldehyde, amino, bromo or iodoacetyl, carboxyl, hydroxyl, epoxy, ester, silane, thiol, and the like.
12. The aggregate of any of paragraphs 1-11, wherein the molecule is hydrophobic, hydrophilic or amphiphilic.
13. The aggregate of any of paragraphs 1-12, wherein the molecule is biologically active.
14. The aggregate of any of paragraphs 1-13, wherein the biological activity is selected from the group consisting of adhesive, polymerization, stimulatory, inhibitory, regulatory, trophic, migratory, toxic, or lethal response in a biological assay.
15. The aggregate of any of paragraphs 1-14, wherein the biological activity is selected from the group consisting of exhibiting or modulating an enzymatic activity, blocking or inhibiting a receptor, stimulating a receptor, modulation of expression level of one or more genes, modulation of cell proliferation, modulation of cell division, modulation of cell migration, modulation of cell differentiation, modulation of cell apoptosis, modulation of cell morphology, and any combinations thereof.
16. The aggregate of any of paragraphs 1-15, wherein the aggregate or the nanoparticle constituent of the aggregate are internalized into a cell
17. The aggregate of any of paragraphs 1-16, wherein said biological activity occurs inside a cell.
18. The aggregate of any of paragraphs 1-17, wherein the aggregate or nanoparticle constituent of the aggregate are biologically active following internalization into the cell.
19. The aggregate of any of paragraphs 1-18, wherein the molecule is a therapeutic agent, or an analog, derivative, prodrug, or a pharmaceutically acceptable salt thereof
20. The aggregate of any of paragraphs 1-19, wherein the therapeutic agent is an antithrombotic agent, a thrombolytic agent, a thrombogenic agent, an anti-inflammatory agent, anti-atherosclerosis agent, anti-infective agent, anti-sepsis agent, anti-cancer agent, an anti-angiogenesis agent, a pro-angiogenesis agent, a vasodilator, a vasoconstrictor, an anti-neoplastic agent, an anti-proliferative agent, an anti-mitotic agent, an anti-migratory agent, an anti-adhesive agent, an anti-platelet agent, or an anti-polymerization agent.
21. The aggregate of any of paragraphs 1-20, wherein the molecule is a plasminogen activator.
22. The aggregate of any of paragraphs 1-21, wherein the plasminogen activator is tissue plasminogen activator (tPA), urokinase, pro-urokinase, streptokinase or plasmin.
23. The aggregate of any of paragraphs 1-22, wherein the molecule is a therapeutic agent and is a monoclonal antibody or fragment thereof or a polyclonal antibody or fragment thereof.
24. The aggregate of any of paragraphs 1-23, wherein the molecule is a diagnostic agent.
25. The aggregate of any of paragraphs 1-24, wherein the molecule is a diagnostic agent and is a monoclonal antibody or fragment thereof or a polyclonal antibody or fragment thereof.
26. The aggregate of any of paragraphs 1-25, wherein the molecule is a targeting ligand.
27. The aggregate of any of paragraphs 1-26, wherein the molecule is a targeting ligand and is a monoclonal antibody or fragment thereof or a polyclonal antibody or fragment thereof.
28. The aggregate of any of paragraphs 1-27, wherein the molecule is an imaging or contrast agent.
29. The aggregate of any of paragraphs 1-28, wherein the imaging or contrast agent is an echogenic substance, a non-metallic isotope, an optical reporter, a fluorescent molecule, a boron neutron absorber, a paramagnetic metal ion, a ferromagnetic metal, a gamma-emitting radioisotope, a positron-emitting radioisotope, or an x-ray absorber.
30. The aggregate of any of paragraphs 1-29, wherein the molecule is a metal or metal oxide comprises a metal selected from the group consisting alkali metals, earth metals, transition metals, post-transition metals, lanthanides, actinides, and any combinations thereof.
31. The aggregate of any of paragraphs 1-30, wherein the aggregate comprises both a therapeutic agent and an imaging or contrast agent.
32. The aggregate of any of paragraphs 1-31, wherein the therapeutic agent is tPA and the imaging or contrast agent is a fluorescent dye.
33. The aggregate of any of paragraphs 1-32, wherein the plurality of nanoparticles comprises a first subpopulation comprising a first type, shape, morphology, size, chemistry, a therapeutic agent, or a imaging or contrast agent and at least one second subpopulation comprising a second type, shape, morphology, size, chemistry, a therapeutic agent, or a imaging or contrast agent, wherein at least one of the first type, shape, morphology, size, chemistry, a therapeutic agent, or a imaging or contrast agent is different from the second type, shape, morphology, size, chemistry, a therapeutic agent, or a imaging or contrast agent.
34. The aggregate of any of paragraphs 1-33, wherein the molecule is a prodrug and the aggregate further comprises a reagent for activating the prodrug.
35. The aggregate of any of paragraphs 1-34, wherein the prodrug is encapsulated within the aggregate.
36. The aggregate of any of paragraphs 1-35, wherein the reagent for activating the prodrug is on outer surface of the aggregate.
37. The aggregate of any of paragraphs 1-36, wherein the reagent for activating the prodrug is covalently linked to the outer surface of the aggregate.
38. The aggregate of any of paragraphs 1-37, wherein the prodrug is on outer surface of the aggregate.
39. The aggregate of any of paragraphs 1-38, wherein the reagent for activating the prodrug is encapsulated in the aggregate.
40. The aggregate of any of paragraphs 1-39, wherein the prodrug is covalently linked to the outer surface of the aggregate.
41. The aggregate of any of paragraphs 1-40, wherein the prodrug is plasminogen and the reagent for activating the prodrug is a plasminogen activator.
42. The aggregate of any of paragraphs 1-41, wherein the plasminogen activator is urokinase, pro-urokinase, streptokinase, plasmin, or tPA.
43. The aggregate of any of paragraphs 1-42, wherein the molecule is released at a higher rate and/or in higher amount from a disaggregated aggregate relative to a non-disaggregated aggregate.
44. The aggregate of any of paragraphs 1-43, wherein aggregate comprises a ligand.
45. The aggregate of any of paragraphs 1-44, wherein the ligand is a targeting ligand.
46. The aggregate of any of paragraphs 1-45, wherein the ligand is selected from the group consisting of peptides; polypeptides; proteins; enzymes; peptidomimetics; antibodies or a portion or fragment thereof; monoclonal antibodies or a portion or fragment thereof; polyclonal antibodies or a portion or fragment thereof; glycoproteins; lectins; nucleosides; nucleotides; nucleic acids; analogues and derivatives of nucleic acids; monosaccharides; disaccharides; trisaccharides; oligosaccharides; polysaccharides; glycosaminoglycans; lipopolysaccharides; lipids; vitamins; steroids; hormones; cofactors; receptors; receptor ligands; and analogs and derivatives thereof.
47. The aggregate of any of paragraphs 1-47, wherein the ligand is selected from the group consisting of CD47 or a fragment thereof, tPA, polylysine (PLL), intercellular adhesion molecules (ICAMS), cellular adhesion molecules (CAMS), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, polyphosphazine, polyethylenimine, cspermine, spermidine, polyamine, pseudopeptide-polyamine, peptidomimetic polyamine, dendrimer polyamine, arginine, amidine, protamine, thyrotropin, melanotropin, lectin, surfactant protein A, mucin, transferrin, bisphosphonate, polyglutamate, polyaspartate, an aptamer, asialofetuin, hyaluronan, procollagen, insulin, transferrin, albumin, acridines, cross-psoralen, mitomycin C, TPPC4, texaphyrin, Sapphyrin, polycyclic aromatic hydrocarbons (e.g., phenazine, dihydrophenazine), bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), RGD peptide, radiolabeled markers, haptens, naproxen, aspirin, dinitrophenyl, HRP, AP, lectins, vitamin A, vitamin E, vitamin K, vitamin B, folic acid, B12, riboflavin, biotin, pyridoxal, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, GalNAc, galactose, mannose, mannose-6P, clusters of sugars such as GalNAc cluster, mannose cluster, galactose cluster, an aptamer, integrin receptor ligands, chemokine receptor ligands, serotonin receptor ligands, PSMA, endothelin, GCPII, somatostatin, and any combinations thereof.
48. The aggregate of any of paragraphs 1-47, wherein the cell adhesion molecule (CAM) is an immunoglobulin, an integrin, a selectin or a cadherin.
49. The aggregate of any of paragraphs 1-48, wherein the aggregate is of a spherical, cylindrical, disc, rectangular, cubical, lenticular or irregular shape.
50. The aggregate of any of paragraphs 1-49, wherein the nanoparticle is of a spherical cylindrical, disc, rectangular, cubical, lenticular or irregular shape.
51. The aggregate of any of paragraphs 1-50, wherein surface of the nanoparticles is modified to modulate intermolecular electrostatic interactions, hydrogen bonding interactions, dipole-dipole interactions, hydrophilic interaction, hydrophobic interactions, van der Waal's forces, and any combinations thereof between two or more nanoparticles.
52. The aggregate of any of paragraphs 1-51, wherein the aggregate is from about 1 μm to about 20 μm in size.
53. The aggregate of any of paragraphs 1-52, wherein the aggregate increases or decreases the in vivo lifetime of the molecule.
54. The aggregate of any of paragraphs 1-53, wherein the aggregate alters biodistribution of the molecule.
55. The aggregate of any of paragraphs 1-54, wherein the nanoparticle comprises at least one moiety that increases the in vivo lifetime of the aggregate.
56. The aggregate of any of paragraphs 1-55, wherein the at least one moiety is polyethylene glycol or CD47 or a fragment thereof
57. The aggregate of any of paragraphs 1-56, wherein the nanoparticles comprises a polymer selected from the group consisting of polysaccharides, polypeptides, polynucleotides, copolymers of fumaric/sebacic acid, poloxamers, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), gelatin, collagen, silk, alginate, cellulose, poly-nucleic acids, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), hyaluron, heparin, agarose, pullulan, and copolymers, terpolymers, and copolymers comprising any combinations thereof.
58. The aggregate of any of paragraphs 1-57, wherein the nanoparticles are liposomes.
59. The aggregate of any of paragraphs 1-58, wherein the nanoparticles are aggregated non-covalently.
60. The aggregate of any of paragraphs 1-59, wherein the aggregate further comprises an aggregating matrix.
61. The aggregate of any of paragraphs 1-60, wherein the aggregating matrix is an excipient, a therapeutic agent, an imaging or contrast agent, or a cleavable linker.
62. A pharmaceutical composition comprising an aggregate of any of paragraphs 1-61 and a pharmaceutically acceptable carrier or excipient.
63. A method of drug delivery to subject, the method comprising administering to the subject an aggregate of any of paragraphs 1-61 or a pharmaceutical composition of paragraph 62, wherein the aggregate comprises a therapeutic agent; and administering a stimulus to the subject to disaggregate the aggregate and thereby controlling release of the therapeutic agent from the aggregate.
64. The method of paragraph 63, wherein the stimulus is selected from the group consisting of ultrasound, mechanical strain, vibration, magnetic field, radiation, temperature, ionic strength, pH, pressure, turbulence, change in flow, flow rate, or chemical or enzymatic activation.

65. A method of treating a vascular stenosis and/or a stenotic lesion and/or an embolic or vasoocclusive lesion in a subject, the method comprising administering to a subject in need thereof an aggregate of any of paragraphs 1-61 or a pharmaceutical composition of paragraph 62.
66. A method of imaging a vascular stenosis and/or a stenotic lesion and/or an embolic or vasoocclusive lesion in a subject, the method comprising administering to a subject in need thereof an aggregate of any of paragraphs 1-61 or a pharmaceutical composition of paragraph 62.
67. The method of paragraph 65 or 66, wherein the stenosis, stenotic or occlusive lesion is selected from the group consisting of arterial occlusive disease; a blood clot; intimal hyperplasia; stent restenosis; intermittent claudication (peripheral artery stenosis); angina or myocardial infraction (coronary artery stenosis); carotid artery stenosi; aortic stenosis, buttonhole stenosis; calcific nodular stenosis; coronary ostial stenosis; double aortic stenosis; fish-mouth mitral stenosis; idiopathic hypertrophic subaortic stenosis; infundibular stenosis; mitral stenosis; muscular subaortic stenosis; subaortic stenosis; subvalvar stenosis; supravalvar stenosis; tricuspid stenosis; renal artery stenosis, mesenteric artery thrombosis; venous stenosis; venous thrombosis; a lesion, disease or disorder of a fluid containing channel; and any combinations thereof.
68. The method of any of paragraphs 65-67, wherein the stenosis, stenotic or occlusive lesion results from trauma or injury, atherosclerosis, cerebral vasospasms, birth defects, diabetes, iatrogenic, infection, inflammation, ischemia, neoplasm, vasospasm, coronary vasospasm, Raynaud's phenomenon, stroke, blood clotting, Moyamoya disease, Takayasu's disease, polyarteritis nodosa, disseminated lupus erythematous, rheumatoid arthritis, tumors of the spine, Paget's disease of bone, fluorosis, hemodialysis, sickle cell anemia, and any combinations thereof.
69. A method of treating internal hemorrhage in a subject, the method comprising administering to a subject in need thereof an aggregate of any of paragraphs 1-61 or a pharmaceutical composition paragraph 62.
70. The method paragraph 69, wherein internal hemorrhage is result of trauma, blood vessel rupture from high blood pressure, infection (e.g., Ebola, Marburg), cancer, scurvy, hepatoma, autoimmune thrombocytopenia, ectopic pregnancy, malignant hypothermia, ovarian cysts, liver cancer, vitamin K deficiency, hemophilia, adverse effect of a medication.
71. A method of theranostic classification in a subject, the method comprising administering to a subject in need thereof an aggregate of any of paragraphs 1-61 or a pharmaceutical composition of paragraph 62, wherein the aggregate comprises a therapeutic agent and a imaging or contrast agent.
72. The method of any of paragraphs 63-71, wherein said administrating is by injection, infusion, instillation, or ingestion.
73. The method of any of paragraphs 63-72, wherein the aggregate is co-administered with a second therapy.
74. The method of any of paragraphs 63-73, wherein the second therapy is an endovascular (e.g., catheter-based) procedure.
75. The method of any of paragraphs 63-74, wherein the second therapy comprises placement of a wire through an occlusion.
76. The method of any of paragraphs 63-75, wherein the second therapy comprises mechanical thrombectomy.
77. The method of any of paragraphs 63-76, wherein the second therapy comprises administering a therapeutic agent for removing or clearing a blood vessel obstruction.
78. The method of any of paragraphs 63-77, wherein the second therapeutic agent is administered at a lower dose than the recommend dose of the second therapeutic agent.
79. The method of any of paragraphs 63-78, wherein the second therapeutic agent is administered before administering of the aggregate.
80. The method of any of paragraphs 63-79, the method further comprising administering a stimulus to the subject to disaggregate the aggregate and thereby controlling release of the therapeutic agent.
81. The method of any of paragraphs 63-80, wherein said stimulus is selected from the group consisting of ultrasound, mechanical strain, magnetic field, radiation, temperature, pressure, change in flow, chemical or enzymatic activation, and any combinations thereof.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. In some embodiments, the term “treatment” can also mean prolonging survival as compared to expected survival if not receiving treatment. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management. The subject is usually ill or injured, or at an increased risk of becoming ill relative to an average member of the population and in need of such attention, care, or management.

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis is actually true. The decision is often made using the p-value.

The term “nanosphere” means a nanoparticle having an aspect ratio of at most 3:1. The term “aspect ratio” means the ratio of the longest axis of an object to the shortest axis of the object, where the axes are not necessarily perpendicular.

The term “longest dimension” of a nanoparticle means the longest direct path of the nanoparticle. The term “direct path” means the shortest path contained within the nanoparticle between two points on the surface of the nanoparticle. For example, a helical nanoparticle would have a longest dimension corresponding to the length of the helix if it were stretched out into a straight line.

The term “nanorod” means a nanoparticle having a longest dimension of at most 200 nm, and having an aspect ratio of from 3:1 to 20:1.

The term “nanoprism” means a nanoparticle having at least two non-parallel faces connected by a common edge.

The “length” of a nanoparticle means the longest dimension of the nanoparticle.

The “width” of a nanoparticle means the average of the widths of the nanoparticle; and the “diameter” of a nanoparticle means the average of the diameters of the nanoparticle.

The “average” dimension of a plurality of nanoparticles means the average of that dimension for the plurality. For example, the “average diameter” of a plurality of nanospheres means the average of the diameters of the nanospheres, where a diameter of a single nanosphere is the average of the diameters of that nanosphere.

As used herein, the term “pharmaceutically-acceptable salts” refers to the conventional nontoxic salts or quaternary ammonium salts of a compound, e.g., from non-toxic organic or inorganic acids. These salts can be prepared in situ in the administration vehicle or the dosage form manufacturing process, or by separately reacting a purified compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

As used herein, a “prodrug” refers to compounds that can be converted via some chemical or physiological process (e.g., enzymatic processes and metabolic hydrolysis) to a an active compound. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

The term “analog” as used herein refers to a compound that results from substitution, replacement or deletion of various organic groups or hydrogen atoms from a parent compound. As such, some monoterpenoids can be considered to be analogs of monoterpenes, or in some cases, analogs of other monoterpenoids, including derivatives of monoterpenes. An analog is structurally similar to the parent compound, but can differ by even a single element of the same valence and group of the periodic table as the element it replaces.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

The term “theranostic” refers to the ability to determine the outcomes of a therapeutic procedure by using diagnostic devices and methods. Theranostics (a portmanteau of therapeutics and diagnostics) is a process of diagnostic therapy for individual patients—to test them for possible reaction to taking a medication and to tailor a treatment for them based on the test results. Theranostics can be a key part of personalized medicine and usually requires considerable advances in predictive medicine, and usually rely on pharmacogenomics, drug discovery using genetics, molecular biology and microarray chips technology. However, the compositions and methods described herein can be used for theranostic purposes without requiring any significant advances in predictive medicine or equipment.

As used herein, the terms “antibody” and “antibodies” refer to intact antibody, or a portion or fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)2, Fv, and single-chain antibodies. The terms “antibody” and “antibodies” include polyclonal antibodies, monoclonal antibodies, humanized or chimeric antibodies, single chain Fv antibody fragments, Fab fragments, and F(ab)2 fragments. Unless it is specifically noted, as used herein a “portion thereof” or “fragment thereof” in reference to an antibody refers to an immunespecific fragment, i.e., an antigen-specific or binding fragment.

Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., Monoclonal Antibodies and Cancer Therapy, Alan R. Liss, Inc., 1983, pp. 77-96). Such antibodies can be of any immunoglobulin class including IgG, IgM, IgE, IgA, IgD, and any subclass thereof. Polyclonal antibodies are heterogeneous populations of antibody molecules that are specific for a particular antigen, which are contained in the sera of the immunized animals. Polyclonal antibodies are produced using well-known methods. A chimeric antibody is a molecule in which different portions are derived from different animal species, such as those having a variable region derived from a murine monoclonal antibody and a human immunoglobulin constant region. Chimeric antibodies can be produced through standard techniques. Antibody fragments that have specific binding affinity for a component of the complex can be generated by known techniques. For example, such fragments include, but are not limited to, F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al., 1989, Science, 246: 1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

In some embodiments the antibody or antigen-binding fragment thereof is recombinant, engineered, humanized and/or chimeric. In some embodiments, the antibody or antigen binding fragment thereof is human.

The antibodies or fragments thereof can be combined with the nanoparticles or aggregates to create therapeutic agents or diagnostic agents. Aggregates and their constituent nanoparticles can comprise on their surfaces both therapeutic and diagnostic antibodies or fragments thereof, which can serve to both identify lesions (e.g., stenoses) and treat said lesions. Alternatively, such antibodies or fragments thereof can serve as ligands to bind the aggregates and their constituent nanoparticles to cell surface receptors/molecules (e.g., proteins, carbohydrates) or extracellular/intercellular molecules.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Nanoparticle Preparation

Nanoparticles (NPs) were prepared from PLGA (50:50,17 kDa, acid terminated; Lakeshore Biomaterials, AL) using a simple solvent displacement method (26). The fluorescent hydrophobic dye, coumarin-6, was included in the NPs to enable visualization and quantitation in this study. Briefly, 1 mg/ml of polymer was dissolved with 0.1 wt % coumarin in dimethyl sulfoxide (DMSO, Sigma, MO), dialyzed against water at room temperature, and the nanoparticles were allowed to form by solvent displacement and subsequent self-assembly in aqueous solution. The size distribution and morphology of the formed NPs were characterized using Dynamic Light Scattering (DLS), Scanning Electron Microscopy (SEM) and Transmission Electron Microscopy (TEM).

Fabrication of SA-NTs—

The PLGA NPs were centrifuged and concentrated to a 10 mg/ml suspension in water and 1 mg/ml L-leucine (Spectrum Chemicals & Laboratory Products, CA) was added. NP aggregates (SA-NTs) were prepared by a spray-drying technique using a Mobile Minor spray dryer (Niro, Inc.; Columbia, Md.). The aqueous leucine-NP suspension was infused separately from the organic phase (ethanol) at a ratio of 1.5:1 and mixed in-line immediately prior to atomization (27). The inlet temperature was 80° C. and the liquid feed rate was 50 ml/min; gas flow rate was set at 25 g/min and nozzle pressure was 40 psi. Spray dried powders were collected in a container at the outlet of the cyclone. SA-NTs suspensions were formed by reconstituting the powders in water at desired concentrations. Aggregate suspensions were filtered through 20 μm filters to filter out any oversized aggregates; centrifugation (2000 g for 5 min) followed by washing also was used to remove single unbound NPs. DLS was used to determine the size of the NPs in dilute solutions using a zeta particle size analyzer (Malvern instruments, UK) operating with a HeNe laser, 173° back scattering detector. Samples were prepared at 1 mg/ml concentration in PBS buffer at pH 7.4. Data collection and analysis was performed with Malvern instrument software.

Functionalization with tPA—

NP aggregates (1 mg/ml) were pre-activated with 1-ethyl-3-(3-dimethylaminopropyl) (EDC) and Sulfo-NHS (N-hydroxysulfosuccinimide) at 1:5:10(PLGA:EDC:NHS) molar ratio in 0.1 M MES buffer, pH 6.0 for 1 hour. The reaction mixture was then centrifuged and washed twice with PBS and subsequently reacted with linker NH2-PEGbiotin (Thermofisher Scientific, Rockford, Ill.) at a 1:10 molar ratio in PBS, pH 7.4 at room temperature for 2 hours. The aggregates were then centrifuged and washed twice and reacted with streptavidin (Thermofisher Scientific, Rockford, Ill.) for 15 minutes at room temperature. The aggregates were purified by repeated centrifugation and washing to remove any unreacted reagents. Separately, human tissue plasminogen activator (tPA, Cell Sciences, MA) was functionalized with biotin using linker NHS—PEG-biotin in PBS at room temperature for 2 hours at a 1:10 molar ratio (23). The functionalized tPA was then reacted with the strepatvidin-biotinaggregates for 30 min at room temperature. The tPA functionalized NP aggregates tPA were then purified by centrifugation and washing; the amount of tPA conjugated to the aggregates was determined by fluorescence spectrometry. Briefly, aggregates were dissolved in 1M NaOH under stirring at 37° C. for ˜6h until a clear polymer solution was obtained. The amount of tPA (TRITC-labeled, Cell Sciences, MA) in the polymer solution was then measured at 594 nm. Activity of tPA coated particles was confirmed using a fluorometric tPA activity assay (SensoLyte, AnaSpec, CA); after immobilization, tPA-coated NPs retained ˜70% of the activity exhibited by soluble tPA. SEM of the aggregated nanoparticles was performed using a Zeiss FESEM Supra55vP (Center for Nanosystems (CNS), Harvard University). Samples were mounted on carbon tape adhesive substrates and sputter coated with gold under vacuum using a sputter coater (Center for Nanosystems (CNS), Harvard University). The coated NP aggregates were imaged at 4 kV using an in-lens detector at 9 mm working distance.

Rheometer Shearing Assay—

A solution of SA-NTs (5 mg/ml in 8% Polyvinylpyrrolidone solution) was sheared for 1 min using a 20 mm cone & plate configuration in a Rheometer (AR-G2 TA Instruments, DE). The solutions were then collected, filtered through a 0.45 micron filter (Millipore, MA) to remove large microscale aggregates from NPs and diluted 1:3 with water. The fluorescence intensity of these NP suspensions was measured using a PTI QM40Fluorometer (PTI-FL) (Photon Technology International, NJ) and normalized relative to the highest shear level (1,000 dyne/cm2) value.

Computational Fluid Dynamics (CFD) Simulations—

CFD simulations for the microfluidic channels were performed using the software package Comsol 3.5 (Comsol, USA), based on a finite element method. We considered the flow to be steady and incompressible, and assumed a no-slip boundary condition at the walls and the fluid medium (PBS) to have a constant densityof 1000 kg/m3 and viscosity of 1 mPa·sec. CFD simulations of IVUS reconstructed blood vessel were performed as previously described (28). Microfluidic Models of Vascular Stenosis-Microchannels mimicking vascular constriction used for studies on microemboli formation were prepared from polydimethylsiloxane (PDMS) using conventional soft lithography (29). A master mold was prepared by aligning 80 micron layers designed using a CAD program and formed using a cutter plotter (CE5000, Graphtec, CA). The device contained a region (160 μm high×400 μm wide×10 mm long) with a 90% constriction relative to upstream and downstream channel regions (each: 640 μm high×2 mm wide×20 mm long). The PDMS channels were sealed with a glass micro slide (170 μm thick) using plasma bonding. In some studies, solutions of SA-NTs (5 ml, 100 ug/ml) were recirculated through microfluidic devices with 90% occlusion or without any constriction using a peristaltic pump (ISM 834C, Ismatec SA, Switzerland). Flow rate was adjusted to obtain a wall shear stress of 10 dyne/cm2 at the unconstructed channels. The suspensions were collected after 20 minutes of flow and filtered through a sub-micron (0.45 um) filter. The fluorescent intensity of the collected NP suspensions was measured using a spectrometer (Photon Technology International, NJ) and normalized relative to the unconstricted channel value. For studies on release of NPs and their binding to endothelial cells in stenotic regions, the microfluidic devices were sterilized using oxygen plasma and coated with fibronectin (50 ug/ml @ 30 min) to support cell adhesion. Bovine aortic endothelial cells (Lonza, MD) were introduced to the microchannel and allowed to adhere under static conditions (2 hr at 37° C.). The devices were then placed in a tissue culture incubator and medium (EGM®-MV BulletKit, Lonza, MD) was infused (50 μL/hr) using a syringe pump (Braintree Scientific, Braintree, Mass.). The endothelial cells were cultured in the devices for 3-4 day until a continuous cell monolayer was formed. A solution containing SA-NTs (10 μg/ml) was then infused for 10 min through the device at a flow rate which produces a wall shear stress of ˜10 dyne/cm2 in the unconstructed channel. Unattached particles were flushed away by infusing water through the channels at the same flow rate for 5 min. Phase contrast and fluorescence microscopic images of cells and bound NPs in regions proximal upstream and downstream to the constriction were acquired using a Zeiss microscope. The averaged fluorescence intensity of cell-associated coumarin loaded NPs obtained from these views was used to evaluate the difference in NP accumulation between pre- and post stenotic regions.

Microfluidic Models of Vascular Embolism—

Microfluidic devices with a narrowed cross-sectional area (80 μm high×0.5 mm wide×200 mm long) were fabricated using soft lithography as described above. Fibrin clots formed as described below that were infused into the main channel lodged and obstructed the flow in these smaller channels. A solution of tPA or tPA coated SA-NTs was infused at a flow rate corresponding to a shear stress of 10 dyne/cm2 in an unobstructed channel. Prior to infusion of the tPA solutions, bovine plasminogen (Cell Sciences, MA) was added to a final concentration of 2.2 μM (30). During the fibrinolysis process, the fibrin clot sizes were monitored in real-time (images acquired every 30 sec) on an inverted Zeiss microscope.

Experimental Fibrin Emboli—

Fibrin clots were formed by adding CaCl2 (20 mM) and human α-thrombin (1 units/ml final concentrations, Enzyme Research Laboratories, IN) to human fibrinogen (5 mg/ml, Enzyme Research Laboratories, IN), as previously described (23, 31). This solution was immediately added drop-wise to a solution of canola oil with Span-80 (0.05%). The emulsion was mixed at 350 rpm for 4 hr and centrifuged (500 g, 5 min), followed by repeated washing in ethanol and water. The diameter of the resulting fibrin beads was determined by optical microscopy to be ˜250 μm. By adjusting the mixing speed in the described protocol, fibrin beads of smaller defined sizes were produced.

Ex-Vivo Mouse Pulmonary Embolism Model—

6-8 week-old C57BL/6 male mice (Jackson Laboratory, Bar Harbor, Me.) were weighed and anesthetized with Avertin (200 mg/kg IP). The trachea was incised via surgical tracheotomy, and cannulated with a blunted 22G stainless steel needle tip. The lungs were subsequently ventilated at a rate of 60 breaths/min, with a Peak Inspiratory Pressure (Pip) of 10 cm H2O and a Positive End Expiratory Pressure (Peep) of 3 cmH2O with compressed air using a mouse ventilator (VCM-R, Hugo Sachs Elektroniks, Germany). Ex vivo ventilation and perfusion of the mouse lung was performed using an IL1 ex vivo mouse lung ventilation-perfusion system (Harvard Apparatus, Natick, Mass.), (32). Following initiation of mechanical ventilation, the chest was opened via thoracotomy, and heparin (100 IU) was injected into the right ventricle. After 30 seconds, the thoracic aorta and superior vena cava were cut and the animal exsanguinated. A suture was placed around the pulmonary artery and aorta. Cannulae made from polyethylene tubing (PE90 (0.86 mm ID, 1.7 mm OD)) were placed in the pulmonary artery (PA) and left atrium (LA), and lungs were perfused with RPMI-1640 with 4% Bovine Albumin (PROBUMIN™ Reagent Grade, Billerica, Mass.) and 0.7 g NaCl/500 ml via a roller pump (ISM 834C, Ismatec SA, Switzerland) set at a constant flow rate of 0.5 ml/min in a recirculating system with a system volume of 6 ml. Perfusate and lung temperatures were maintained at 37° C. by housing the entire ex-vivo ventilation perfusion system inside a standard cell incubator without CO2 (Forma Scientific, Ohio). Humidity was maintained in the range 09°-95%. Pulmonary arterial and left atrial pressures and airway flow and pressures were recorded with dedicated Type 379 vascular pressure and DLP2.5 flow and MPX Type 399/2airway pressure transducers and TAM-A amplifiers (Hugo Sachs Elektroniks, Germany). Vascular pressures were zeroed at the mid lung level prior to each experiment and recorded using Polyview16© software (Grass Technologies, West Warwick, R.I.). Prior to injection of experimental fibrin clots (prepared as above), the measured pressures were allowed to stabilize and remain stable for a period of more than 10 minutes. A solution of fibrin clots suspended in perfusion medium was infused at a flow rate of 0.1 ml/min and mixed with the regular perfusion line entering the pulmonary artery. The fibrin clot suspension was infused until the pulmonary artery pressure increased to ˜three-fold higher than the baseline. The system was then allowed to equilibrate and remain stable for at least 10 minutes. Next, tPA-coated SA-NTs or free soluble tPA was added to the main perfusion line and circulated through the perfusion system. As in the microfluidic device experiments, when tPA was perfused, plasminogen was added to obtain a final concentration of 2.2 μM. Pulmonary artery and vein pressures were acquired continuously during the perfusion period. At the end of each experiment, lungs were perfused with 4% paraformaldehyde, and prepared for sectioning by incubating in 4% paraformaldehyde, then sucrose and OCT. All experimental animal protocols were approved by the Institutional Animal Care and Use Committee at Children's Hospital Boston and Harvard Medical School.

In Vivo Mouse Pulmonary Embolism Model (PE)—

6-8 week-old male C57BL/6 mice (Jackson Laboratory, Bar Harbor, Me.) were weighed and anesthetized with Avertin (200 mg/kg IP). A ventro-lateral incision was made in the neck and a jugular vein catheter (PE10 tubing (0.28 mm ID, 0.61 mm OD, BD Biosciences) was inserted through which a solution containing preformed fibrin clots was infused using a syringe pump (Braintree Scientific Inc). Different size of emboli were used: in the acute PE model, large fibrin clots were used (150±80 micron; 0.1 ml @ 1×103 clots/ml over 2 minutes), while in the peripheral PE model smaller emboli were injected (30±25 micron; 0.1 ml@ 1×104 clots/ml over 2 minutes). Animals were subsequently injected via the jugular vein catheter with tPA coated SA-NTs (1 mg particles/ml in PBS @ 3 ul/min; 500 ng tPA total) or with carrier fluid for 45 minutes. Following the treatment period, animals were further monitored for an additional 15 min. Animal core temperature was maintained at 37° C. using a temperature regulated heating lamp. At the conclusion of the experiments animals were euthanized and their lungs and organs prepared for histology using standard techniques. Prior to histology, intact lungs were placed under an upright fluorescent microscope and emboli located in peripheral blood vessel were observed. Bright field and fluorescent images of the lungs were captured. The size distribution of emboli observed in the peripheral blood vessels was determined using a threshold and performing size analysis on the fluorescent images (ImageJ) on areas which were in focus in their corresponding bright field image.

Mouse Ferric Chloride Arterial Injury Model—

A previously described model was used with minor modifications (16). In brief, male C57BL/6 mice (3-4 weeks old) were anesthetized with 2.5% tribromoethanol (0.15 ml/10 g) and injected with fluorescently labeled platelets (calceinRed/Orange, ˜1×109 platelets/kg). An incision was made through the abdominal wall to expose the mesentery and arterioles (˜100 μm in diameter) were visualized using a Zeiss Axiovert 135-inverted microscope (objectives: 10× and 32×, Carl Zeiss Microlmaging, Inc.) and recorded on videotape. Whatman filter paper saturated with FeCl3 (10%) solution was applied topically for 5 min, which caused denudation of the endothelium. 100 μl of PBS solutions with either SA-NTs coated with tPA (50 ng tPA, 1 mg), bare SA-NTs, soluble tPA (50 ng), pre-dispersed t-PA SA-NTs into NPs (pre-sheared using 30 min flow in microfluidic devices with wall shear stress of 1,000 dyne/cm2, followed by sonication @60 W, 2 min), unbreakable shear insensitive SA-NTs coated with t-PA (NPs in SA-NTs were fused by incubation in 60° C. for >4 hr) or PBS alone were administrated through the retro-orbital plexus of the eye, 7-8 min after removal of the ferric chloride filter paper. Following this bolus injection, the vessels were monitored until full occlusion occurred (blood flow stopped) and lasted for more than 10 seconds. The shear rate was calculated using an optical Doppler velocity meter (Microcirculation Research Institute, Texas A&M College of Medicine, College Station, Tex.) (17). One arteriole was chosen per mouse.

Adhesion of NPs and Microaggregates Under Flow—

Microfluidic devices contain a narrowed channel (80 μm high×2 mm wide×200 mm long) were fabricated using soft lithography as described above. A glass slide coated with a thin dry layer of fibrin (<1 μm thick) was bonded to the bottom of the channel. Fluorescent NPs (200 nm) and microparticles (2 μm) were coated with tPA as detailed above. A solution of the coated NPs or microaggregates (100 ug/ml) was infused in the channel at a flow rate corresponding to a wall shear stress of 10 dyne/cm2 for 15 min. At the end of the experiment, the channels were washed with water at the same flow rate for >10 min. Fluorescence microscopy images were taken and analyzed to evaluate the area covered by particles.

Biodistribution of Particles in Mice—

100 μl of SA-NTs or dispersed NPs solution (5 mg/ml) was bolus injected through the jugular vein of anesthetized male 6-8 week-old C57BL/6 mice. Five minutes post injection animals were killed and the major organs (liver, lungs, spleen and kidney) were harvested. The organs were homogenized in DMSO and mixed for 30 min on a shaker. The mixed solutions were then centrifuged (10,000 g for 10 min) and the supernatant was collected. The fluorescence intensity of the supernatant was measured using PTI QM40Fluorometer (PTI-FL), (Photon Technology International, NJ) at 460/515 nm excitation and emission. Organs from control mice were similarly processed, and the baseline organ autofluorescence values measured were subtracted from the treated group measurements. A calibration curve built using SA-NTs solutions of different concentrations was used to correlate the tissue measurements to their injected dose (ID) values. Accumulation of particles in the blood was estimated by fluorescent intensity measurements of blood samples.

Disruption of normal blood flow to the heart, lung and brain is the leading cause of death and long-term adult disability in the western world (1). Current approaches to acutetherapy for ischemic stroke, coronary infarction, and pulmonary embolism require infusion of thrombolytic drugs, which need to be administered systemically or through a catheter placed within the obstructed vessel, usually in an acute care hospital setting (2-4). To be effective, patients must receive therapy within a few hours after onset of symptoms, and the doses of clot-lysing drugs that can be administered are limited by the potential risk of bleeding as active drug is free to distribute throughout the body. To overcome these limitations, inventors designed an athrombolytic delivery system that targets drugs selectively to sites of flow obstruction and concentrates active drug in these regions.

Stenotic and thrombosed blood vessels exhibit unique physical characteristics that distinguish them from normal vasculature in that fluid shear stress can increase locally by one to two orders of magnitude, from below approximately 70 dyne/cm2 in normal vessels to greater than 1,000 dyne/cm2 in highly constricted arteries (5-8). Normal circulating platelets are locally activated by high shear stress in these regions and rapidly adhere to the adjacent surface lining of the narrowed vessels (9-11), which is a major contributing factor in development of vulnerable atherosclerotic plaques. Inspired by this natural physical mechanism of platelet targeting, we developed a therapeutic strategy that uses local high shear stress as a generic mechanism to target treatment to regions of blood vessels that are constricted by clots, stenosis or developmental abnormalities.

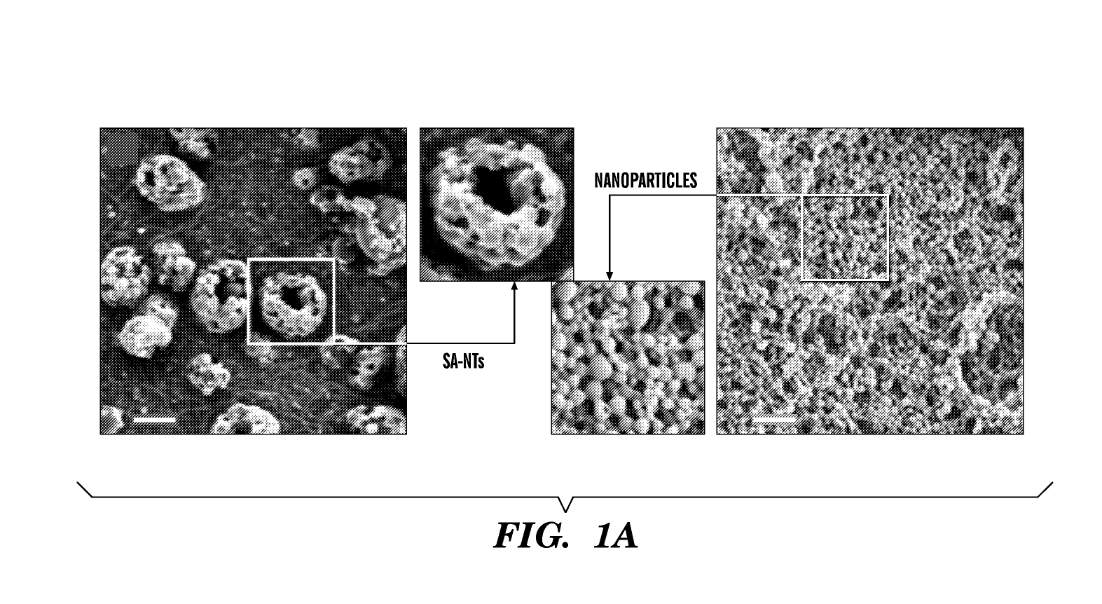

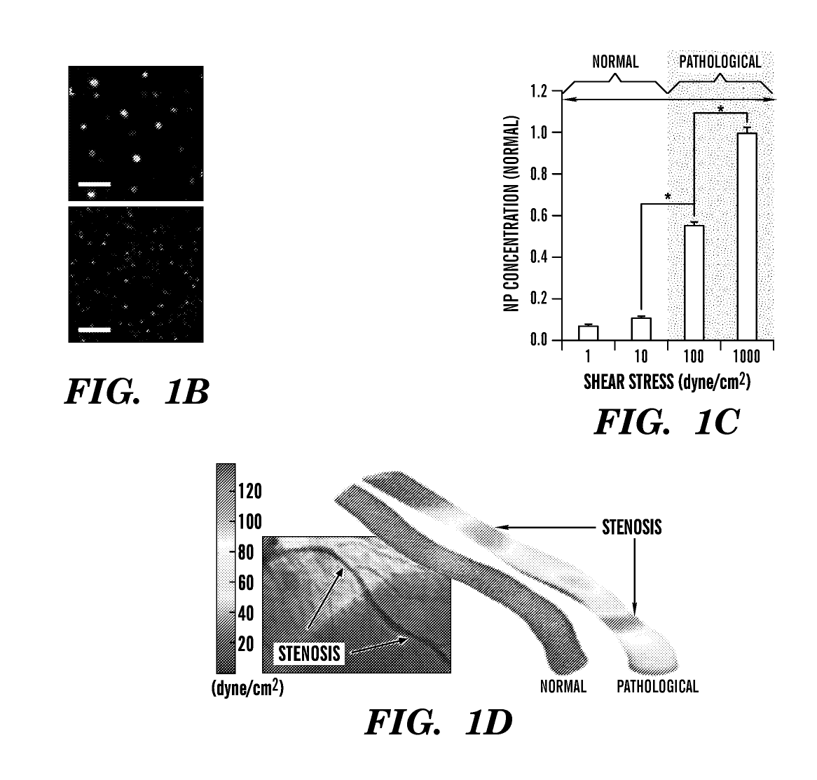

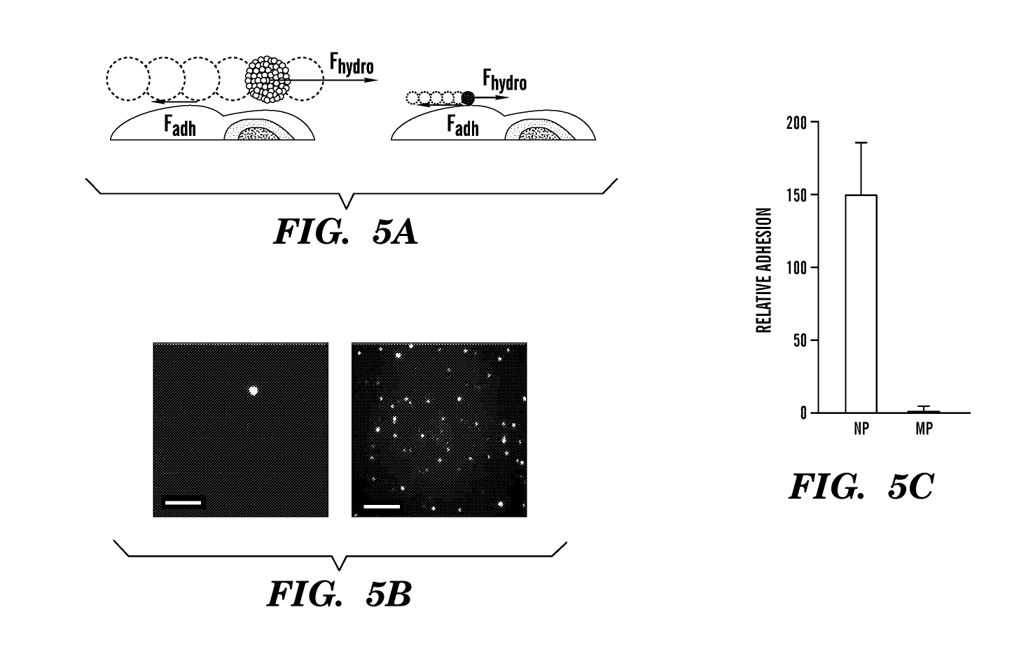

Our shear-activated nanotherapeutics (SA-NTs) are similar in size to natural platelets (1 to 5 μm in diameter); however, they are fabricated as aggregates of multiple smaller nanoparticles (NPs). The microscale aggregates remain intact when flowing in blood underphysiological flow conditions, but break up into individual nanoscale components when exposed to high local shear stress. Because of their smaller size compared to the microscale aggregates, shear-dispersed NPs experience lower drag forces and hence, they adhere more efficiently to the surface of the adjacent blood vessel wall than the larger microaggregates (FIG. 5). The efficiency of this local adhesion can be further enhanced by coating the NPs with molecules that bind to endothelial cells or relevant targets, such as fibrin clots. In this manner, high concentrations of therapeutic agents can be concentrated locally at sites of vascular occlusion or embolism by immobilizing relevant drugs or enzymes on the NPs. The SA-NTs were produced by spray-drying concentrated solutions of biocompatible, biodegradable, poly-lactic-co-glycolic acid (PLGA 50:50, MW 17 kDa) to form micrometer-sized (3.8±1.6 μm) aggregates composed of small (180±70 nm) NPs (FIG. 1A). Microaggregates of PLGA NPs are stable in aqueous solutions due to their hydrophobicity (12, 13). But when exposed to mechanical forces that overcome the attractive forces holding the NPs together, such as hemodynamic shear stresses, the aggregates break apart (FIG. 1B), much like a wet ball of sand disperses into individual grains when rubbed in one's hands.

To determine the shear-sensitivity of this NP deployment mechanism, a rheometer was used to apply controlled shear stresses in vitro to SA-NTs fabricated from NPs labeled with a fluorescent tag. We detected an 8- to 12-fold increase in the concentration of released NPs when the level of shear reached 100 dyne/cm2 or higher (FIG. 1C). This range of fluid shear stress is relevant in many vascular diseases. For example, computational fluid dynamics (CFD) modeling of flow within normal and stenotic human left coronary arteries based on ultrasound imaging (see Methods; 15, 16) revealed that the level of shear that induce NP release in vitro is similar to that generated by a 60% lumen obstruction (FIG. 1D) whereas normal coronary vessels experience a 5-fold lower level of shear stress (˜10 to 30 dyne/cm²) that does not cause disruption of the SA-NTs.

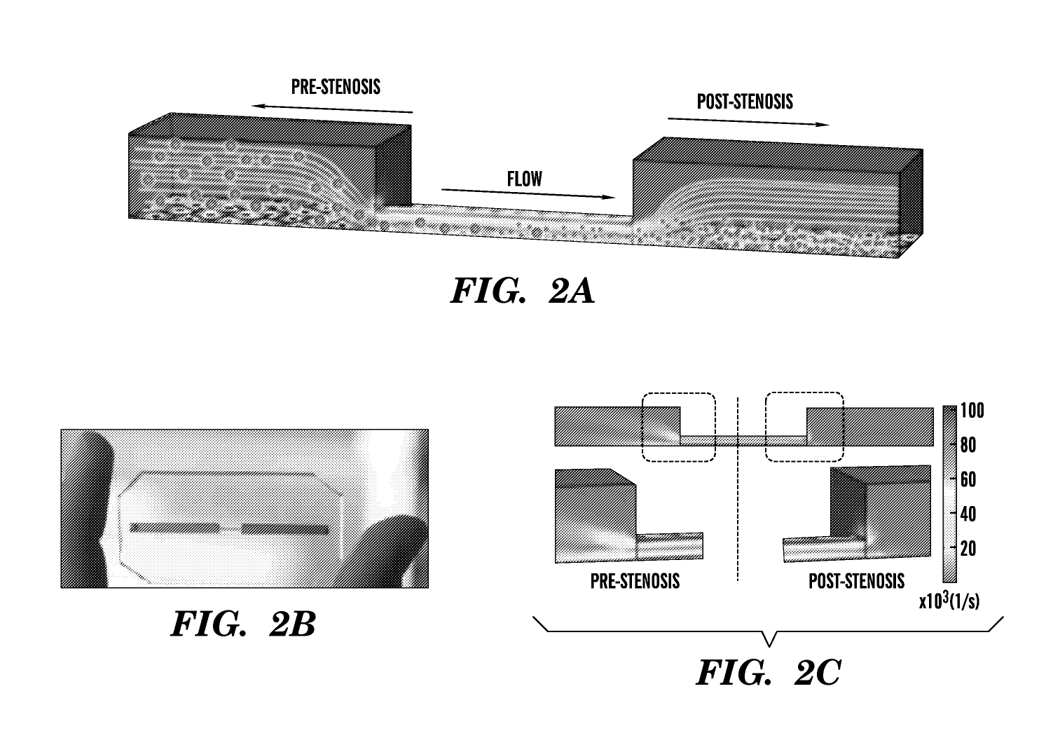

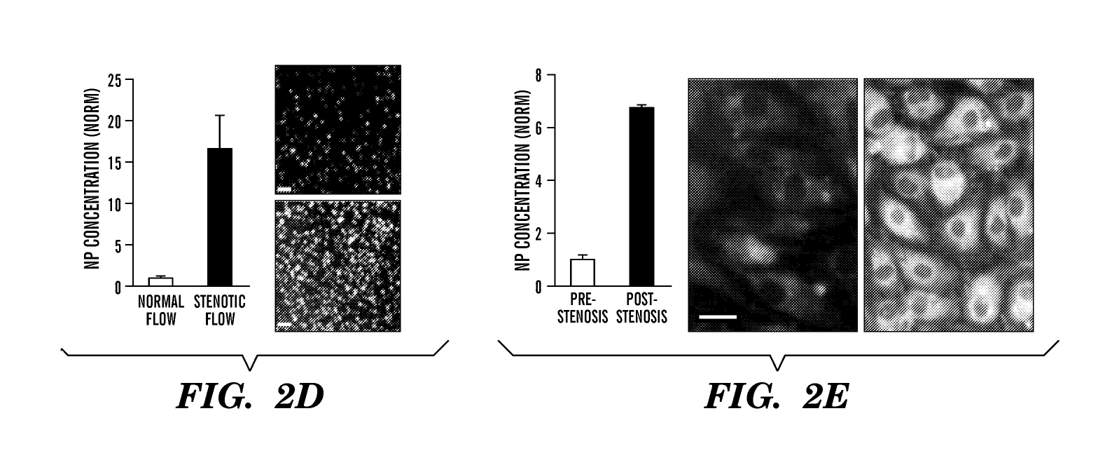

To determine whether these SA-NTs can target agents selectively to stenotic regions under relevant hemodynamic flow conditions, we carried out studies in a three-dimensional (3D) microfluidic model of vascular narrowing fabricated from poly-dimethylsiloxane (PDMS) that was designed to mimic regions of living blood vessels with 90% lumen obstruction (FIGS. 2A and B). Based on CFD modeling, such a constriction generates ˜100 fold increase in shear at the stenotic site (FIG. 2C). Perfusion of SA-NTs (100 μg/ml in PBS) through these microfluidic devices resulted in a 16-fold increase in the release of free NPs, as measured in the solution flowing downstream of the obstruction compared to fluid flowing through unobstructed microfluidic channels of similar dimensions (FIG. 2D). Moreover, fluorescence microscopic imaging confirmed that released NPs accumulated in endothelial cells cultured on the inner surface of the artificial microfluidic vessel just distal to the narrowed region whereas minimal uptake occurred in cells lining the channel prior to the constriction (FIG. 2E).

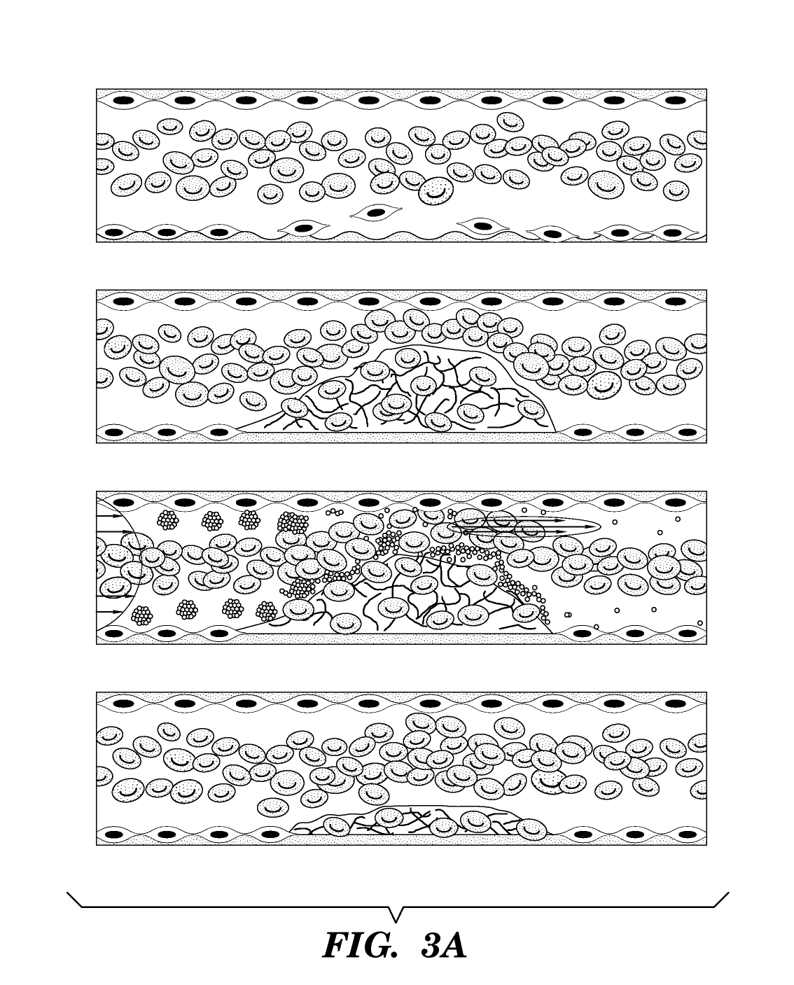

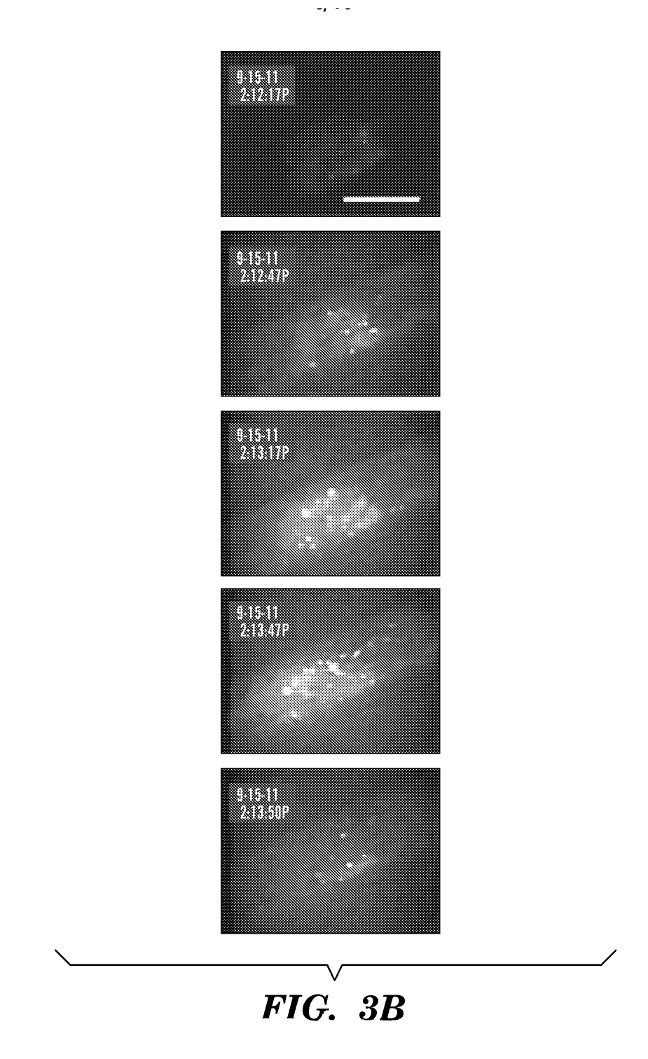

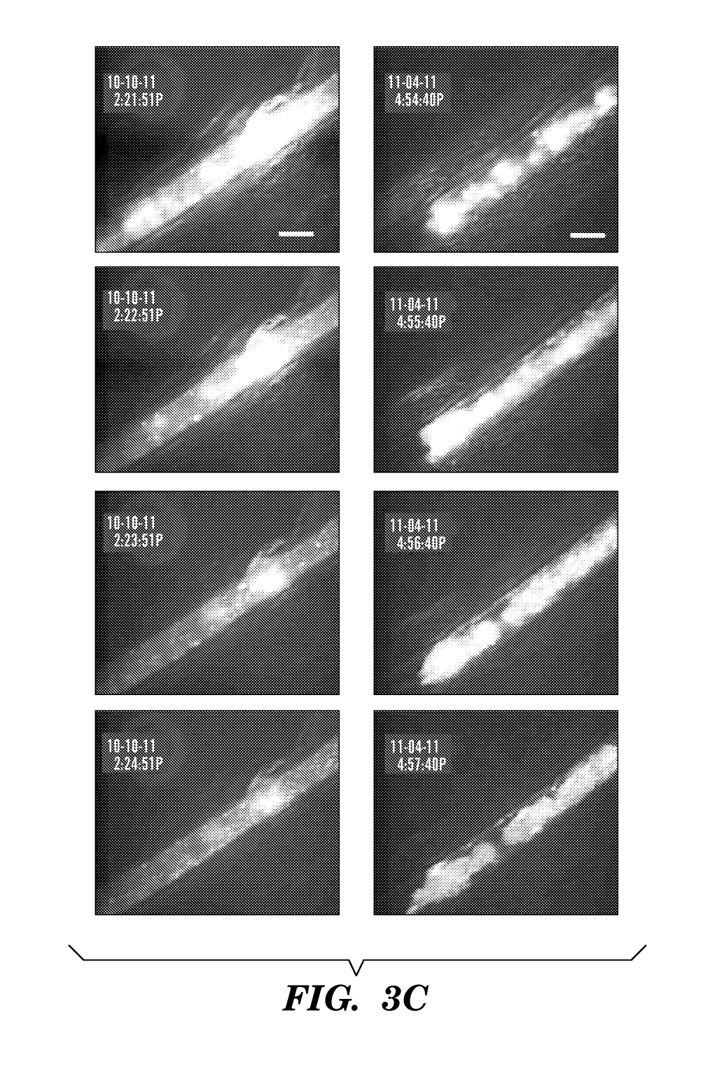

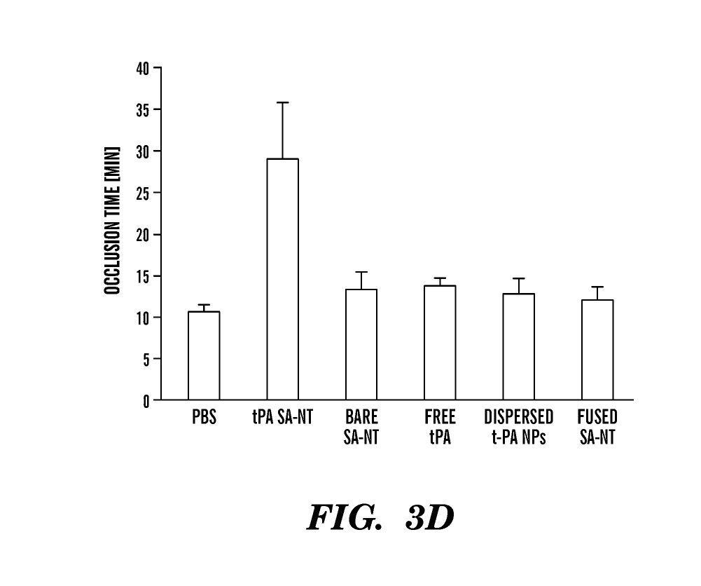

To evaluate their functional potential, we fabricated SA-NTs containing fluorescent NPs coated with the FDA-approved thrombolytic drug, tissue plasminogen activator (tPA), using biotin-streptavidin chemistry (˜5×105 tPA molecules/micro-aggregate) and tested their ability to dissolve blood clots. To examine the general utility of this shear-targeted nanotherapeutic approach (FIG. 3A) for removal of natural clots formed endogenously in vivo, we studied the effect of bolus injection of thrombolytic SA-NTs in an established mouse arterial thrombus model in which clot formation is triggered by injuring the vessel wall by direct exposure to ferric chloride (14-16). Real-time, intravital, fluorescence microscopic studies confirmed that this treatment resulted in formation of large blood clots within minutes in injured mesenteric arteries (˜100 μm diameter, normal wall shear stress ˜30 dyne/cm2 (17) that occluded the diameter by more than 80% (FIGS. 3B and 3C) and caused the local shear stress to increase by more than 15-fold (˜450 dyne/cm2) in these regions, as determined using an optical Doppler velocity meter. Fluorescently-labeled tPA-carrying SA-NTs that were injected intravenously 8 min after chemical injury preferentially accumulated in the regions of clot formation, resulting in clear microscopic visualization of these lesions (FIG. 3B). In addition, the locally deployed tPA-coated NPs induced progressive surface erosion of the thrombi, with complete clearance of occlusions occurring within 5 min after SA-NT injection (FIGS. 3B and 3C). Continuous monitoring of unobstructed vessels for up to 15 minutes in the mesenteric bed revealed that intact microscale NP aggregates continued to be observed throughout the course of the study, confirming that circulation of the SA-NTs through the normal vasculature did not induce microaggregate disruption.

Importantly, shear-induced release of tPA-coated NPs from the SA-NTs reopened the obstructed mesenteric arteries and significantly delayed the time to vessel occlusion (29±7 min with tPA-coated SA-NTs versus 12±3 min with PBS), when vessel patency was monitored using intravenous injection of fluorescently-labeled platelets (˜2.5% of total platelets) (FIG. 3C,D). In contrast, when loaded with the same tPA dose, neither addition of free tPA, pre-dissociated tPA-NPs, nor heat-fused tPA-NP microaggregates (that do not dissociate in high shear) produced any detectable effects in this model (FIG. 3D). Careful analysis of these results also revealed that even when a vessel is almost fully occluded, the microscale tPA-coated SA-NTs that bind to the surface of the clot can actively degrade and ‘recanalize’ the clot. Once this happens, flow and shear stress rapidly increase once again, and this feeds back to activate other tPA carrying SA-NTs, resulting in full clot removal (FIG. 3C). Taken together, these results provide proof-of-principle that the SA-NT technology can be used to target clot-lysing agents to vascular occlusions, in addition to providing a way to image these lesions in real-time in situ.

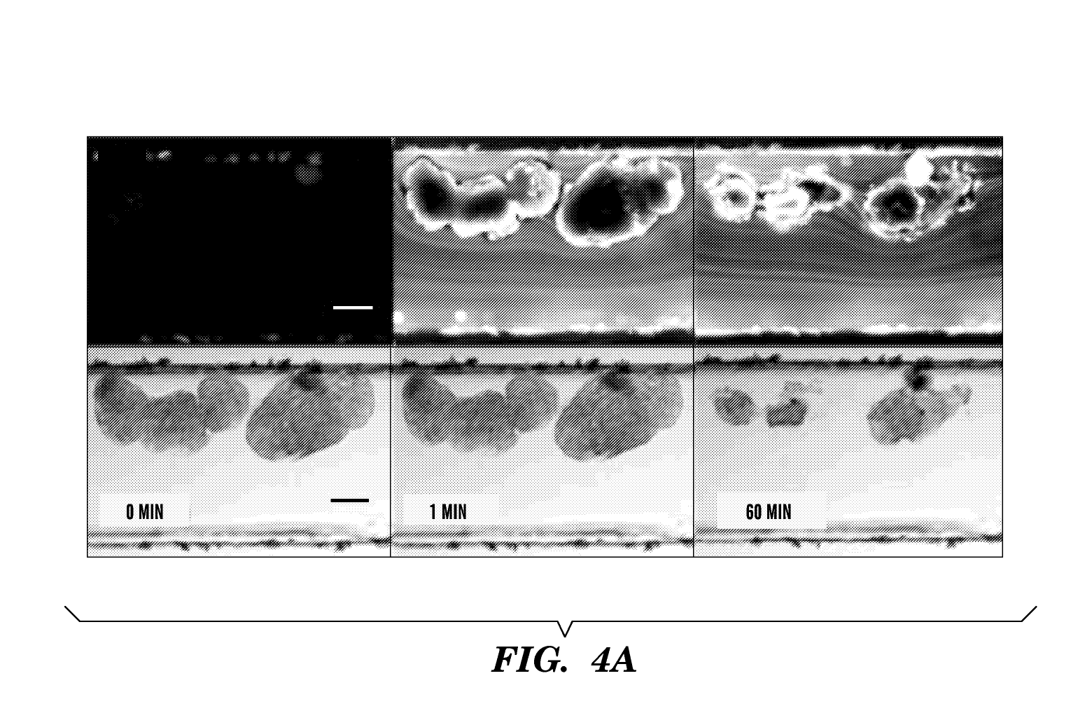

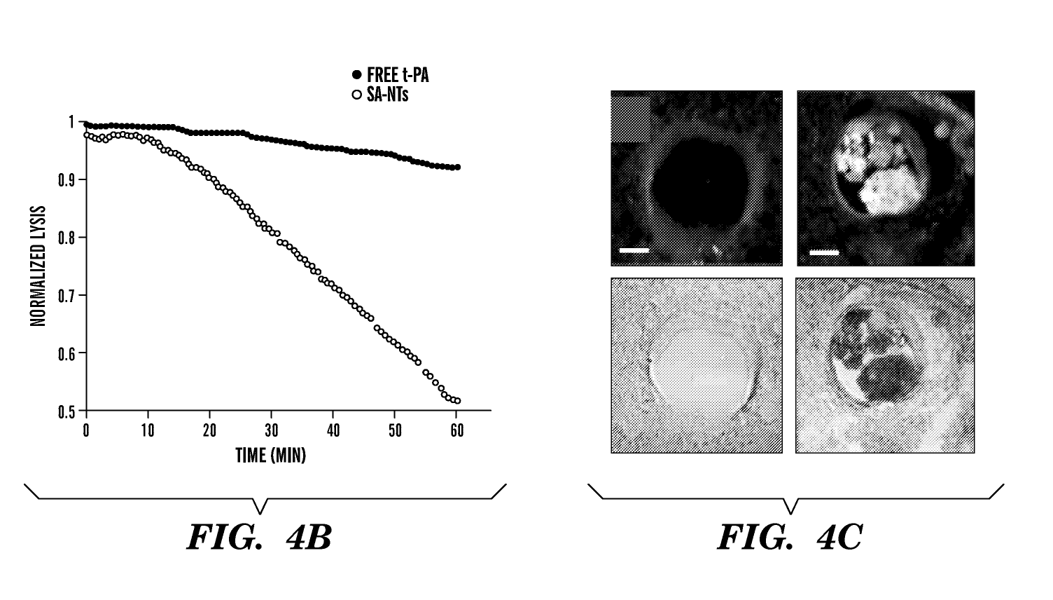

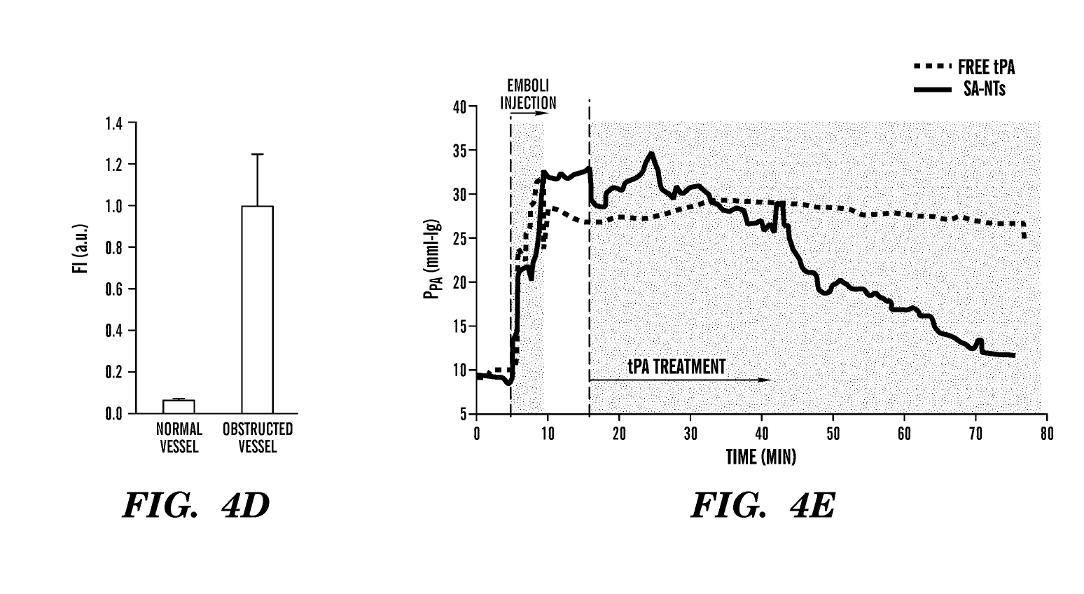

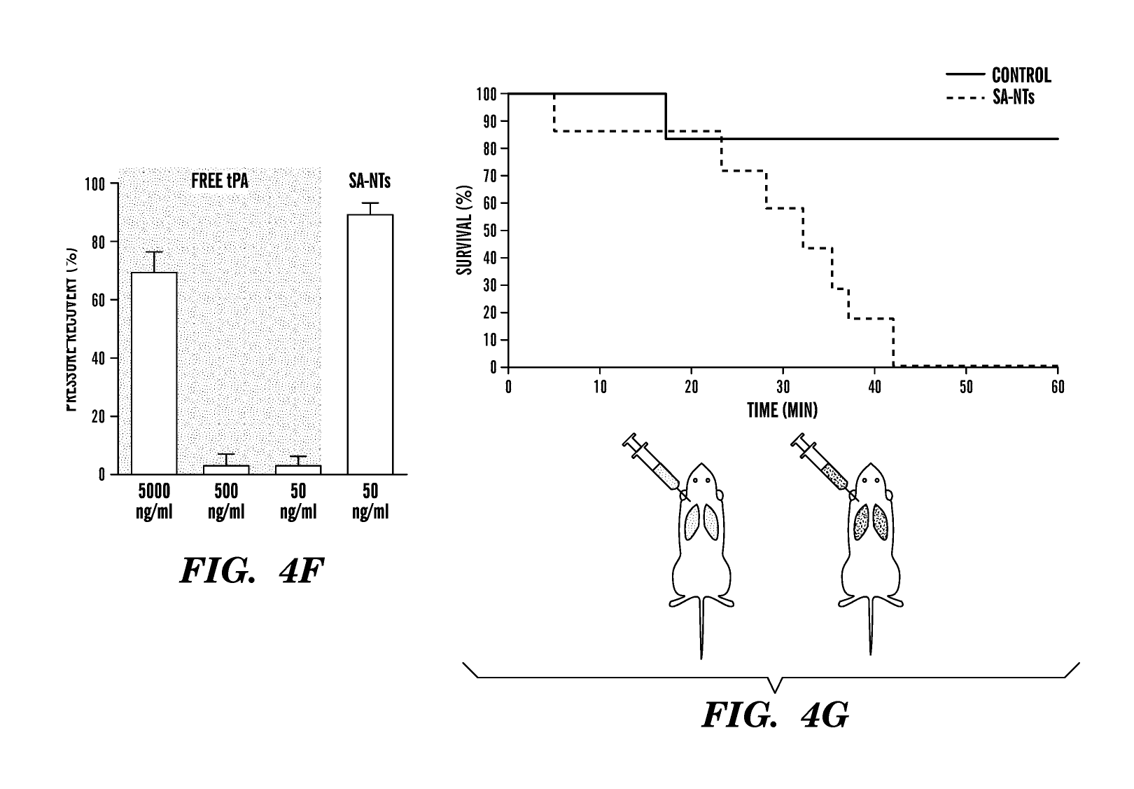

To explore the potential value of the SA-NTs for treatment of life-threatening embolic occlusions, we first tested their ability to dissolve experimentally induced fibrin clots in vitro. When pre-formed fibrin clots (250±150 μm diameter produced by a water-in-oil emulsion technique; (18)) were injected into microfluidic channels that contained constricted regions (80□m high, 500 μm wide), the fibrin emboli lodged in the devices and partially obstructed flow in the channels (FIG. 4A). When SA-NTs (100 μg/ml) carrying tPA (50 ng/ml) were infused at physiological flow rates through the clot-occluded microfluidic channels, the shear-dispersed fluorescent tPA-coated NPs accumulated at the surface of the artificial emboli, progressively dissolving the clots and reducing their size by one half within an hour of treatment (FIG. 4A). In contrast, treatment with soluble tPA at the same concentration and flow conditions had negligible effects (<5% reduction in clot size; FIG. 4B).

Next, we tested the ability of this shear-activated tPA delivery system to reverse the effects of acute pulmonary embolism in an ex vivo whole mouse lung ventilation-perfusion model. A solution containing the pre-formed fibrin clots similar to those tested in the microfluidic channel were infused (0.1 ml/min for ˜5 min; 1×103 clots/ml) through the pulmonary artery of the perfused lung. Occlusion of pulmonary blood vessels by multiple microemboli (FIG. 4C) caused the pulmonary artery pressure to increase by about 3-fold compared to its normal value (30 versus 8 mm Hg; FIG. 4E). We then perfused tPA-coated SA-NTs (100 μg/ml microscale aggregates containing NPs coated with 50 ng/ml tPA) through the pulmonary artery at a physiological flow rate (0.5 ml/min). Fluorescence microscopic analysis of tissue sections again confirmed that the tPA-NPs localized selectively at regions of vascular occlusion, producing a greater than 25-fold increase in accumulation of NPs at these sites, (FIGS. 4C,D). Progressive lysis of the emboli by the tPA-NPs resulted in normalization of pulmonary artery pressure levels within 1 hour in this ex vivo model (FIG. 4E). In contrast, perfusion of soluble tPA at the same concentration as that delivered on the injected tPA-coated NPs (50 ng/ml), or even at a ten times higher dose (500 ng/ml), failed to produce any significant response (FIG. 4F). In fact, similar clot-lysing effects and hemodynamic changes were only observed when we administered a hundred times higher concentration of soluble tPA under identical flow conditions (FIG. 4F); this dose in mice (˜2 mg/kg) is comparable to the therapeutic dose commonly used in humans (˜1 mg/kg).

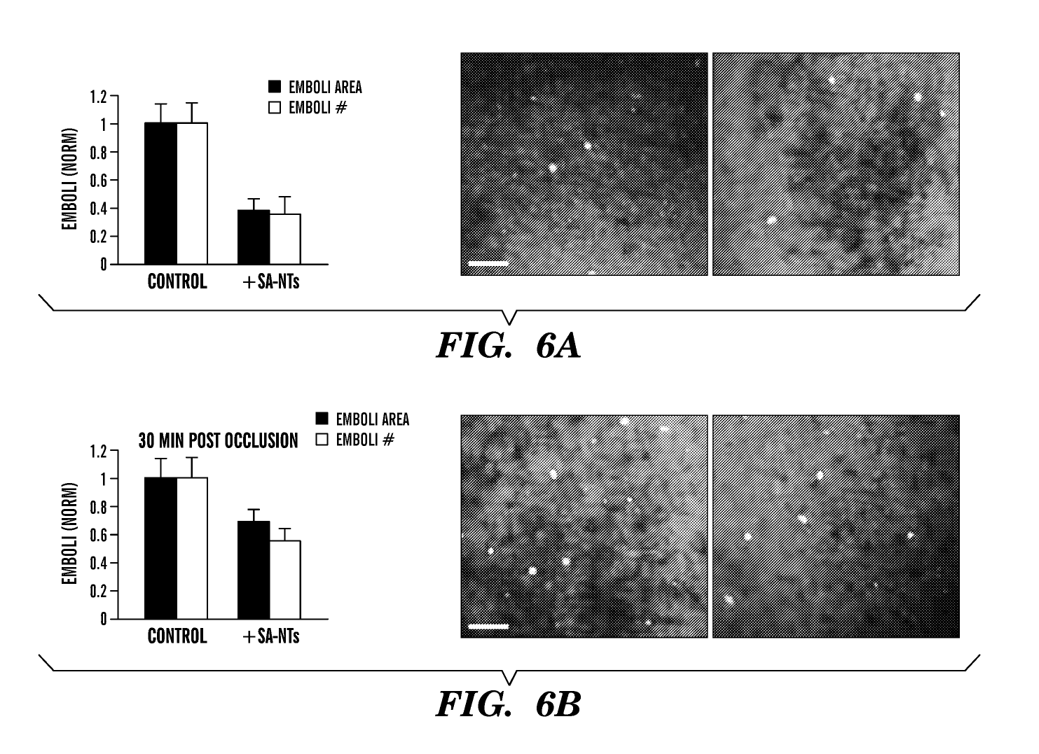

We then studied pulmonary embolism in living mice by infusing smaller preformed fluorescent fibrin clots (<70 μm; ˜1,000 clots) into the jugular vein of anesthetized mice, which accumulate in peripheral blood vessels in the lungs, as previously described (19). SA-NTs coated with tPA were then infused either immediately after injection of emboli, or 30 min after they formed. Quantitation of the total area of fluorescent emboli visualized in the lungs using computerized image analysis confirmed that administration of the tPA-coated SA-NTs resulted in reduction of both total clot area and clot number by more than 60% when administered immediately after injection of emboli, and by more than 30% when infused one half hour after embolism (FIG. 6). To further examine the potential clinical relevance of our approach for treatment of life-threatening acute massive embolism, we infused a solution containing larger fibrin clots (150±80 μm diameter; ˜100/injection), which accumulate in the main pulmonary arteries (19) much as they do in humans with pulmonary embolism, and the mice were then immediately infused with tPA-coated SA-NTs or with carrier fluid for 45 min. All control animals died within 1 hr after infusion of the clots (0% survival, n=7, FIG. 4G), whereas more than 80% of the treated mice survived (6 out of 7), and none of these SA-NTs-treated animals displayed any visible symptoms of respiratory distress.

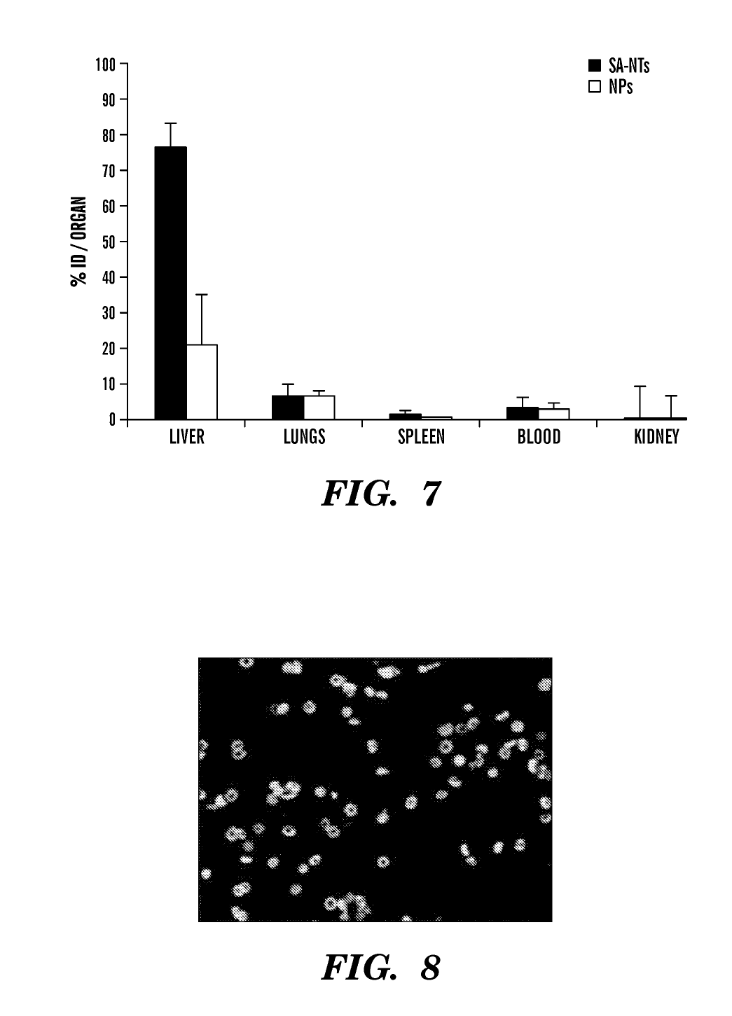

The major potential advantage of the SA-NTs is their ability to enhance the safety of thrombolytic therapies by significantly reducing the drug dose required to be effective, as demonstrated by the ability of SA-NTs to clear pulmonary emboli when coated with a tPA dose˜ 1/100th that required for induction of similar clot-lysing effects by free tPA. SA-NTs also could help to minimize unwanted bleeding and neurotoxicity because they are cleared rapidly from the circulation (80% clearance in 5 min FIG. 7), and due to their larger size, they should not diffuse as easily into injured tissues as free tPA. Finer control over the size of the microscale aggregates and their pharmacokinetics can be used to ensure that they safely pass through all microvessels and are sustained in the circulation at effective levels (20). Alternative methods to link tPA to NPs (e.g., direct conjugation by amine-carboxylate coupling or coupling based on biocompatible heterobifunctional PEG linkers; (21, 22)) can be used to avoid immune responses associated with streptavidin/biotin conjugation and to increase conjugation efficiency as well as optimize tPA activity.

A previously described thrombo-prophylaxis strategy based on coupling plasminogen activators to carrier erythrocytes has shown promising results in preventing thrombosis in various animal models (23-25). The SA-NTs described here can be used to prevent formation of thrombi that partially occlude vascular flow, as occurs for example when a stable atherosclerotic plaque is transformed into a life-threatening vulnerable plaque. However, in contrast to the erythrocyte delivery approach that is limited to prevention of nascent clot formation, the shear-activated drug targeting strategy described herein also offers the ability to treat and dissolve pre-existing fibrin clots, such as those found in patients with stroke and myocardial infarction as well as atherosclerosis. It is also important to note that in addition to delivering tPA in this study, the SA-NTs used were also loaded with a fluorescent dye, which was also effectively localized to these sites. Thus, it is possible to design and fabricate SA-NTs containing various drugs or imaging agents for localized treatment and real-time visualization in a wide variety of pathologies associated with vascular obstruction.

In summary, these findings provide an example of a safer and more effective therapeutic strategy. In contrast to drug targeting mechanisms that focus on expression of distinct molecular species that can vary between tissues or patients, shear stress increases as a function of narrowing of the lumen diameter in all patients, regardless of the cause or location of obstruction, thus offering a robust and broadly applicable targeting strategy. The shear-activated drug targeting nanotechnology described here can be used for immediate administration of clot-busting drugs to patients suspected to have life-threatening clots in the brain, lung or other vital organs by emergency technicians or other care-givers, even before the patient has reached a hospital setting.

### REFERENCES FOR EXAMPLE 1

1. C. J. L. Murray, A. D. Lopez, The Lancet 349, 1269 (1997).
2. T. J. Ingall et al., Stroke 35, 2418 (2004).
3. T. G. Kwiatkowski et al., New England Journal of Medicine 340, 1781 (1999).
4. L. R. Wechsler, New England Journal of Medicine 364, 2138 (2011).
5. J. Strony, A. Beaudoin, D. Brands, B. Adelman, American Journal of Physiology-Heartand Circulatory Physiology 265, H1787 (1993).
6. D. M. Wootton, D. N. Ku, Annual review of biomedical engineering 1, 299 (1999).
7. J. M. Siegel, C. P. Markou, D. N. Ku, S. Hanson, Journal of biomechanical engineering 116, 446 (1994).
8. D. L. Bark Jr, D. N. Ku, Journal of biomechanics 43, 2970 (2010).
9. Z. M. Ruggeri, J. N. Orje, R. Habermann, A. B. Federici, A. J. Reininger, Blood 108, 1903 (2006).
10. W. S. Nesbitt et al., Nature medicine 15, 665 (2009).
11. S. Goto et al., Circulation 99, 608 (1999).
12. M. Santander-Ortega, A. Jódar-Reyes, N. Csaba, D. Bastos-González, J. Ortega-Vinuesa, Journal of colloid and interface science 302, 522 (2006).
13. M. Santander-Ortega, N. Csaba, M. Alonso, J. Ortega-Vinuesa, D. Bastos-González, Colloids and Surfaces A: Physicochemical and Engineering Aspects 296, 132 (2007).
14. H. Ni et al., Journal of Clinical Investigation 106, 385 (2000).
15. A. K. Chauhan et al., The Journal of experimental medicine 203, 767 (2006).
16. W. Bergmeier et al., Proceedings of the National Academy of Sciences 103, 16900(2006).
17. P. S. Frenette et al., Blood 91, 1318 (1998).
18. R. Gref et al., Science 263, 1600 (1994).
19. J. C. Murciano et al., American Journal of Physiology-Lung Cellular and Molecular Physiology 282, L529 (2002).
20. J. A. Straub, D. E. Chickering, T. G. Hartman, C. A. Gloff, H. Bernstein, International journal of pharmaceutics 328, 35 (2007).
21. M. Di Marco et al., International journal of nanomedicine 5, 37 (2010).
22. S. Rana, Y. C. Yeh, V. M. Rotello, Current opinion in chemical biology 14, 828 (2010).
23. J. C. Murciano et al., Nature biotechnology 21, 891 (2003).
24. K. Danielyan et al., Circulation 118, 1442 (2008).
25. K. Ganguly et al., Journal of Pharmacology and Experimental Therapeutics 316, 1130(2006).
26. C. E. Astete, C. M. Sabliov, Journal of Biomaterials Science, Polymer Edition 17, 247(2006).
27. J. C. Sung et al., Pharmaceutical research 26, 1847 (2009).
28. A. U. Coskun et al., Catheterization and cardiovascular interventions 60, 67 (2003).
29. Y. Xia, G. M. Whitesides, Annual review of materials science 28, 153 (1998).
30. S. L. Diamond, Annual review of biomedical engineering 1, 427 (1999).
31. C. K. Lam, T. Yoo, B. Hiner, Z. Liu, J. Grutzendler, Nature 465, 478 (2010).
32. D. Huh et al., Science 328, 1662 (2010).

### Example 2

### Shear Stress Controlled Release from RBCs

Red blood cells ghosts were prepared using hypotonic hemolysis method. In brief, RBC were centrifuged from blood (2000 g, 10 min) and resuspended in calcium/magnesium free diluted PBS (PBS to DD water vol ratio of 1:10). The cells were allowed to incubate for 15 minutes at 4° C. and then centrifuged (12,000 g, 10 min). This process was repeated four times. Afterwards the cells were loaded with FITC-dextran by incubating the cells with 5 mg/ml dextran in diluted PBS for 1 hour at 4° C. The cells were centrifuges, suspended in PBS buffer with Ca/Mg and allowed to reseal in a 37° C. incubator for more than 2 hr. Following the resealing procedure the cells were washed in PBS for four times to remove any residuals in solution. FIG. 8 shows a fluorescence image of RBC ghosts loaded with FITC-dextran taken five days after preparation of FITC-dextran loaded ghosts.

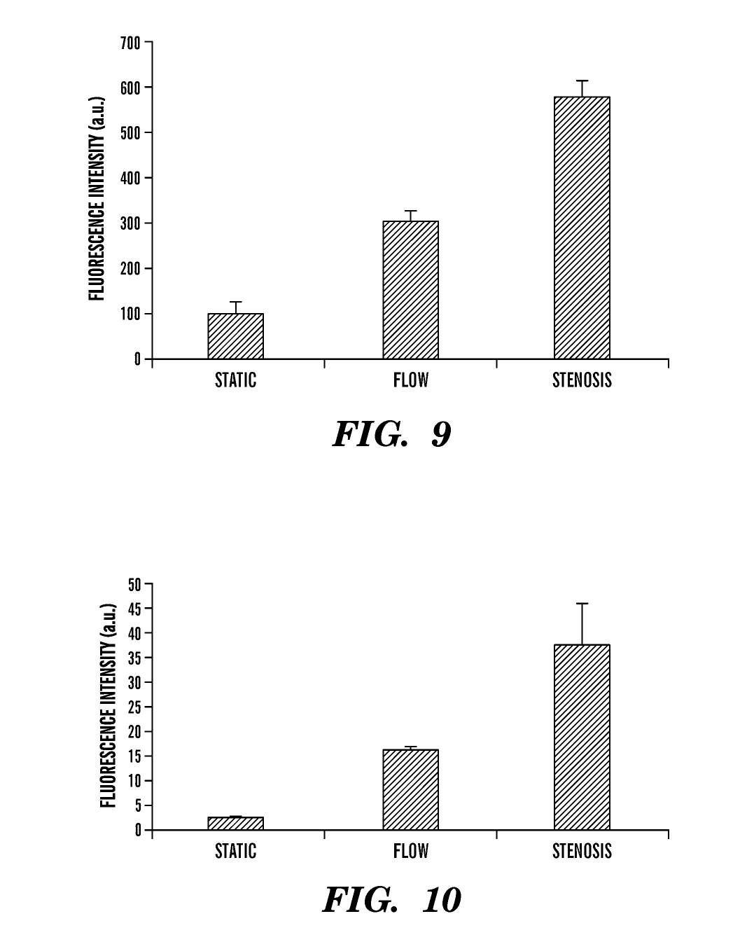

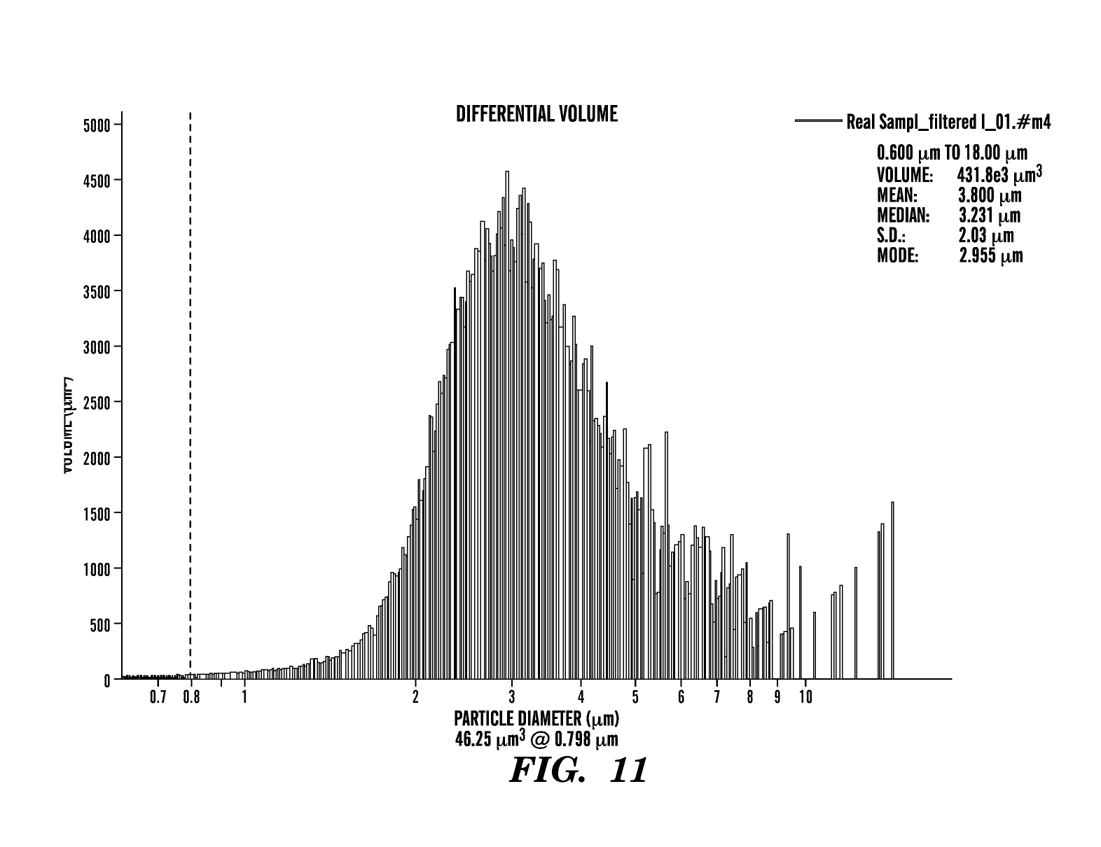

A suspension of FITC-dextran loaded RBC ghosts was infused through a device without a stenosis region (640 micron height channel, wall shear stress 10 dyne/cm²) or with a stenosis region (80% stenosis, 80 micron in height). The suspension was then centrifuged and filtered through a 0.22 μm filter to remove RBCs and the fluorescence intensity was measured. As shown in FIG. 9, the flow induced release was more than two fold higher with the stenosis compared to without the stenosis.

### Example 3

### Shear Stress Controlled Release from Microcapsules

For nanocapsules, Pluronic/poly(ethylenimine) (F127/PEI) nanocapsules encapsulating rhodadmine dye was prepared by emulsification/solvent evaporation with slight modification from a previously reported method (S. H. Choi, S. H. Lee & T. G., Park, Temperature-sensitive pluronic/poly(ethylenimine) nanocapsules for thermally triggered disruption of intracellular endosomal compartment, Biomacromolecules. 2006 June; 7(6):1864-70). Briefly, Pluronic F127 was activated with p-nitrophenyl chloroformate in tolune for 24 hours at room temperature. The product was precipitated in ether and characterized by 1H NMR. To prepare the nanocapsules, 30% of activated F127 and a small amount of hydrophobic dye (rhodamine) was dissolved in dichloromethane (1 ml) and then added dropwise into a 10 ml aqueous PEI solution (7.5 w/v, pH 9). The mixture was stirred at room temperature for about an hour to obtain nano/micro capsules and to evaporate the entrapped dichloromethane. The obtained microcapsules were then purified either by centrifugation or neutralized and dialyzed against water at pH 4.

A suspension of Pluronic-PEI microcapsules loaded with FICT dextran (70 kDa) was infused through a device without a stenosis region (640 micron height channel, wall shear stress 10 dyne/cm²) or with a stenosis region (80% stenosis, 80 micron in height). The suspension was then centrifuged and filtered through a 0.22 μm filter to remove the microcapsules and the fluorescence intensity was measured. As shown in FIG. 10, the flow induced release was more than two fold higher with the stenosis compared to without the stenosis.

### REFERENCES

1. R. Langer, Nature 392, 5 (1998).
2. V. P. Torchilin, European Journal of Pharmaceutical Sciences 11, S81 (2000).
3. T. Mammoto, D. E. Ingber, Development 137, 1407 (2010).
4. N. Resnick et al., Endothelium 10, 197 (2003).
5. J. J. Hathcock, Arteriosclerosis, Thrombosis, and Vascular Biology 26, 1729 (2006).
6. O. K. Baskurt, Biorheology 45, 629 (2008).
7. J. N. Topper, M. A. Gimbrone Jr, Molecular Medicine Today 5, 40 (1999).
8. S. A. Berger, L. D. Jou, Annual Review of Fluid Mechanics 32, 347 (2000).
9. J. Strony, A. Beaudoin, D. Brands, B. Adelman, American Journal of Physiology-Heart and Circulatory Physiology 265, H1787 (1993).
10. Z. M. Ruggeri, J. N. Orje, R. Habermann, A. B. Federici, A. J. Reininger, Blood 108, 1903 (2006).
11. O. C. Farokhzad et al., Anal. Chem 77, 5453 (2005).
12. C. E. Astete, C. M. Sabliov, Journal of Biomaterials Science, Polymer Edition 17, 247 2006).
13. J. C. Sung et al., Pharmaceutical Research 26, 1847 (2009).
14. N. Tsapis, D. Bennett, B. Jackson, D. A. Weitz, D. A. Edwards, Proceedings of the National Academy of Sciences of the United States of America 99, 12001 (2002).
15. K. Avgoustakis, Current Drug Delivery 1, 321 (2004).
16. K. C. Koskinas et al., Circulation 121, 2092 (2010).
17. P. H. Stone et al., European Heart Journal 28, 705 (2007).
18. T. G. Kwiatkowski et al., New England Journal of Medicine 340, 1781 (1999).
19. J. C. Murciano et al., Nature Biotechnology 21, 891 (2003).
20. V. F. Tapson, New England Journal of Medicine 358, 1037 (2008).
21. C. J. L. Murray, A. D. Lopez, The Lancet 349, 1269 (1997).
22. T. J. Ingall et al., Stroke 35, 2418 (2004).
23. T. G. Kwiatkowski et al., New England Journal of Medicine 340, 1781 (1999).
24. J. Strony, A. Beaudoin, D. Brands, B. Adelman, American Journal of Physiology-Heart and Circulatory Physiology 265, H1787 (1993).
25. D. M. Wootton, D. N. Ku, Annual review of biomedical engineering 1, 299 (1999).
26. J. M. Siegel, C. P. Markou, D. N. Ku, S. Hanson, Journal of biomechanical engineering 116, 446 (1994).
27. Z. M. Ruggeri, J. N. Orje, R. Habermann, A. B. Federici, A. J. Reininger, Blood 108, 1903 (2006).
28. W. S. Nesbitt et al., Nature medicine 15, 665 (2009).
29. S. Goto et al., Circulation 99, 608 (1999).
30. R. C. Sonntag, W. B. Russel, Journal of colloid and interface science 113, 399 (1986).
31. H. A. Stone, Annual Review of Fluid Mechanics 26, 65 (1994).
32. R. Gref et al., Science 263, 1600 (1994).
33. V. F. Tapson, New England Journal of Medicine 358, 1037 (2008).
34. S. G. Soriano, S. A. Lipton, Nature medicine 4, (1998).
35. B. E. Figueroa, R. F. Keep, A. L. Betz, J. T. Hoff, J. H. Garcia, Stroke 29, 1202 (1998).
36. C. D. Murray, The Journal of General Physiology 9, 835 (1926).
37. M. Zamir, The Journal of General Physiology 69, 449 (1977).
38. N. Resnick et al., Endothelium 10, 197 (2003).
39. J. J. Hathcock, Arteriosclerosis, thrombosis, and vascular biology 26, 1729 (2006).
40. O. K. Baskurt, Biorheology 45, 629 (2008).
41. J. N. Topper, M. A. Gimbrone Jr, Molecular medicine today 5, 40 (1999).
42. J. A. Champion, A. Walker, S. Mitragotri, Pharmaceutical research 25, 1815 (2008).
43. O. C. Farokhzad et al., Anal. Chem 77, 5453 (2005).
44. R. Wiewrodt et al., Blood 99, 912 (2002).
45. S. H. Choi, S. H. Lee & T. G., Park, Biomacromolecules, 7(6):1864-70 (2006).

### Example 4

### Nanoparticle Aggregates for Drug Targeting Using Ultrasound

The shear activated micro-aggregates can also be dispersed into nanoparticles and deliver drug to specific areas in the body when exposed to an ultrasound stimulus. Compared to other ultrasound drug delivery methods, which are based on rupture of micro-bubbles or liposomes, the method disclosed herein are based on dispersing nanoparticles to release the molecule of interest (e.g. drug). This allows: use of lower intensities of ultrasound versus high intensity ultrasound used to break up the micro-bubbles/liposome which requires complex equipment and can cause local tissue damage and would be too harmful for non-cancer or non-acute treatments. Further, this also allows controlled release of the drug from the nanoparticle over time as opposed to the burst release from current proposed carriers. Moreover, this also allows combining targeting moieties on the nanoparticles. Generally, the nanoparticles are not ruptured.

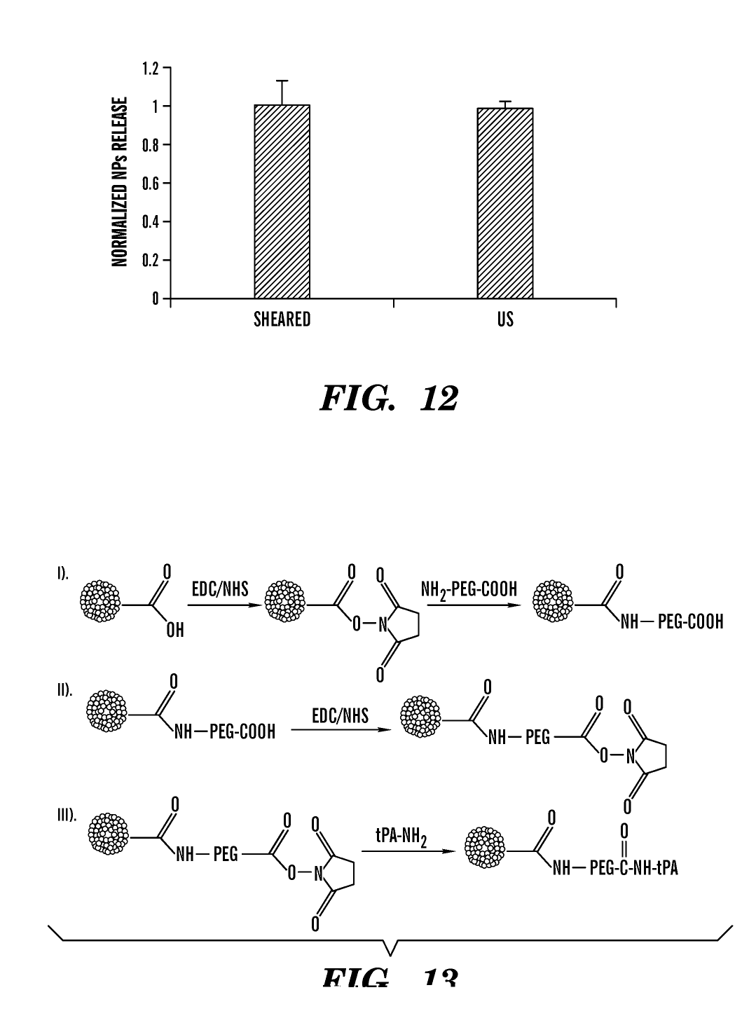

FIG. 12 demonstrates the ability of a clinical therapeutic ultrasound to disperse the aggregates into nanoparticles similarly to the dispersion through a stenotic narrowing by shear (1,000 dyne/cm²). Briefly, 10 ml of micro-particle suspension (0.5 mg/ml) were placed in a 10 cm petri dish. The acoustic agitation was applied using a clinical therapeutic ultrasound device (Sonicator 730—Mettler Electronics, Anaheim, Calif.), used for physiotherapy. A 2 W/cm⁻² intensity at the transducer and a 1 MHz pulsed signal with a 50% duty cycle was used. The suspensions were collected after and filtered through a sub-micron (0.45 um) filter.

### Example 5

### PEGylation Based Conjugation of tPA at the Surface of PLGA Microaggregates

A PEGylation based approach has been selected to replace biotin/streptavidin conjugation chemistry so as to coat tPA on the PLGA particles. The idea was to make the system clinically relevant by using a biocompatible strategy. Each step of the chemistry approach is depicted on the FIG. 13. First, carboxylic groups on the PLGA nanoparticles are activated by EDC/NHS chemistry. Subsequently, the heterobifunctional amino PEG acid is conjugated to the particles via a coupling between amines and activated carboxylic groups. These carboxylic groups are then activated by EDC/NHS chemistry before being conjugated to tPA via tPA amine groups. All the purification steps are done by dialysis or centrifugation/washing.

This method successfully yielded a tPA binding efficiency of 23±2 and 28±4%, respectively with the use of PEG_{10,000} and PEG_{3,400}. The tPA binding efficiency is twice as much as higher compared to the previous biotin/streptavidin approach (<10%). In the case of the highest binding efficiency (with PEG_{3,400}), the grafting density of the PEGylation has been evaluated by the use of a rhodamine-PEG_{3,400}-NH₂ and has shown that 65% of PLGA carboxylic groups was conjugated to the rhodamine PEG. As the carboxylic groups are not exclusively located on the surface of the particles, it can be assumed that the surface of the particles is extensively covered. The activity of tPA is not affected by this new method of grafting and remains higher than 90% after conjugation as compared with the activity of control tPA (data based on SensoLyte® AMC tPA Activity Assay, AnaSpec, Inc. and Human Tissue Plasminogen Activator Activity ELISA Assay, Cell Sciences, Inc.).

This method can be used for conjugating other molecules, e.g., drugs, on the surface of PLGA nanoparticles, based on the use of linear or branched heterobifunctional PEG with different molecular weights.

Content of all patents and other publications identified herein is expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An aggregate comprising a plurality of nanoparticles, wherein the aggregate disaggregates under a predetermined stimulus selected from the group consisting of ultrasound, mechanical strain, vibration, magnetic field, radiation, temperature, ionic strength, pH, pressure, turbulence, change in flow, flow rate, or chemical or enzymatic activation.

2-81. (canceled)

82. The aggregate of claim 1, wherein the aggregate further comprises a molecule selected from the group consisting of small or large organic or inorganic molecules; carbon-based materials; metals; metal oxides; complexes comprising metals; inorganic nanoparticles; metal nanoparticles; monosaccharides; disaccharides; trisaccharides; oligosaccharides; polysaccharides; glycosaminoglycans; biological macromolecules; enzymes; amino acids; peptides; proteins; peptide analogs and derivatives thereof; peptidomimetics; antibodies and portions or fragments thereof; lipids; carbohydrates; nucleic acids; polynucleotides; oligonucleotides; genes; genes including control and termination regions; self-replicating systems; nucleic acid analogs and derivatives; an extract made from biological materials; naturally occurring or synthetic compositions; or any combinations thereof.

83. The aggregate of claim 2, wherein the molecule is absorbed/adsorbed on the surface of the aggregate or the nanoparticle constituent of the aggregate.

84. The aggregate of claim 2, wherein the molecule is encapsulated in the aggregate or the nanoparticle constituent of the aggregate.

85. The aggregate of claim 2, wherein the molecule is covalently linked to the aggregate or the nanoparticle constituent of the aggregate.

86. The aggregate of claim 2, wherein the aggregate or the nanoparticle constituent of the aggregate comprises a surface reactive group for linking with the molecule.

87. The aggregate of claim 2, wherein the molecule is biologically active.

88. The aggregate of claim 87, wherein the biological activity is selected from the group consisting of adhesive, polymerization, stimulatory, inhibitory, regulatory, trophic, migratory, toxic, or lethal response in a biological assay.

89. The aggregate of claim 87, wherein the biological activity is selected from the group consisting of exhibiting or modulating an enzymatic activity, blocking or inhibiting a receptor, stimulating a receptor, modulation of expression level of one or more genes, modulation of cell proliferation, modulation of cell division, modulation of cell migration, modulation of cell differentiation, modulation of cell apoptosis, modulation of cell morphology, and any combinations thereof.

90. The aggregate of claim 87, wherein said biological activity occurs inside a cell.

91. The aggregate of claim 2, wherein the molecule is a therapeutic agent, or an analog, derivative, prodrug, or a pharmaceutically acceptable salt thereof.

92. The aggregate of claim 91, wherein the therapeutic agent is an antithrombotic agent, a thrombolytic agent, a thrombogenic agent, an anti-inflammatory agent, anti-atherosclerosis agent, anti-infective agent, anti-sepsis agent, anti-cancer agent, an anti-angiogenesis agent, a pro-angiogenesis agent, a vasodilator, a vasoconstrictor, an anti-neoplastic agent, an anti-proliferative agent, an anti-mitotic agent, an anti-migratory agent, an anti-adhesive agent, an anti-platelet agent, or an anti-polymerization agent.

93. The aggregate of claim 91, wherein the molecule is a plasminogen activator.

94. The aggregate of claim 2, wherein the molecule is a targeting ligand.

95. The aggregate of claim 2, wherein the aggregate comprises both a therapeutic agent and an imaging or contrast agent.

96. The aggregate of claim 2, wherein the molecule is a prodrug and the aggregate further comprises a reagent for activating the prodrug.

97. The aggregate of claim 1, wherein the aggregate further comprises an aggregating matrix.

98. A method of drug delivery to subject, the method comprising administering to the subject an aggregate of claim 1, wherein the aggregate comprises a therapeutic agent; and administering a stimulus to the subject to disaggregate the aggregate and thereby controlling release of the therapeutic agent from the aggregate.

99. A method of treating or imaging a vascular stenosis and/or a stenotic lesion and/or an embolic or vasoocclusive lesion in a subject, the method comprising administering to a subject in need thereof an aggregate of claim 1.

100. The method of claim 99, wherein the aggregate is co-administered with a second therapy.

101. The method of claim 100, wherein the second therapy is an endovascular procedure.

102. The method of claim 100, wherein the second comprises placement of a wire through an occlusion, mechanical thrombectomy, or administering a therapeutic agent for removing or clearing a blood vessel obstruction.

103. A method of treating internal hemorrhage in a subject, the method comprising administering to a subject in need thereof an aggregate of claim 1.

104. A method of theranostic classification in a subject, the method comprising administering to a subject in need thereof an aggregate of claim 1, wherein the aggregate comprises a therapeutic agent and a imaging or contrast agent.
